(19) United States
(12) Patent Application Publication
(10) Pub. No.: US 2024/0009323 A1
(43) Pub. Date: Jan. 11, 2024

(54) METHODS OF TREATING COLON CANCER USING NANOPARTICLE MTOR INHIBITOR COMBINATION THERAPY

(71) Applicant: Abraxis BioScience, LLC, Summit, NJ (US)

(72) Inventor: Neil P. DESAI, Pacific Palisades, CA (US)

(21) Appl. No.: 18/134,293

(22) Filed: Apr. 13, 2023

Related U.S. Application Data
(63) Continuation of application No. 16/224,449, filed on Dec. 18, 2018, now abandoned.
(60) Provisional application No. 62/607,798, filed on Dec. 19, 2017.

(51) Int. Cl. A61K 47/69; C07K 16/22; A61K 31/436; A61P 35/00; A61K 9/00; A61K 9/51; A61K 31/555; A61K 45/06; A61K 47/64; A61K 39/395; A61K 31/519; A61K 31/513

(52) U.S. Cl. CPC A61K 47/6925; A61K 47/6907; C07K 16/22; A61K 31/436; A61P 35/00; A61K 9/0019; A61K 47/6929; A61K 9/5169; A61K 31/555; A61K 45/06; A61K 47/643; A61K 39/395; A61K 31/519; A61K 31/513; A61K 39/3955; B82Y 5/00

(57) ABSTRACT

The present application provides methods of treating a colon cancer (such as advanced and/or metastatic colon cancer) in an individual, comprising administering to the individual: a) an effective amount of a composition comprising nanoparticles comprising an mTOR inhibitor (such as a limus drug, such as sirolimus or a derivative thereof) and an albumin, b) an effective amount of anti-VEGF antibody (such as bevacizumab), and c) a therapeutically effective FOLFOX regimen (such as FOLFOX4 or a modified FOLFOX6).

### CROSS-REFERENCE TO RELATED APPLICATIONS

This application is a continuation of U.S. patent application Ser. No. 16/224,449, filed Dec. 18, 2018, which claims priority benefit of U.S. Provisional Application No. 62/607,798, filed Dec. 19, 2017, the disclosure of which is incorporated herein by reference in its entirety.

### FIELD OF THE INVENTION

This invention pertains to methods and compositions for the treatment of a colon cancer by administering compositions comprising nanoparticles that comprise an mTOR inhibitor (such as a limus drug, e.g., sirolimus or a derivative thereof) and an albumin in combination with an anti-VEGF antibody and a FOLFOX regimen.

### BACKGROUND OF THE INVENTION

Colon cancer (colorectal cancer, CRC) is a major health concern worldwide due to its high prevalence and mortality rate. In developed countries, it is the third most common malignancy and the second most common cause of cancer-related death. Although advances in the treatment of CRC have made a major impact on its management, many patients with advanced disease will eventually die as a result of their cancer.

The mammalian target of rapamycin (mTOR) is a conserved serine/threonine kinase that serves as a central hub of signaling in the cell to integrate intracellular and extracellular signals and to regulate cellular growth and homeostasis. Activation of the mTOR pathway is associated with cell proliferation and survival, while inhibition of mTOR signaling leads to inflammation and cell death. Dysregulation of the mTOR signaling pathway has been implicated in an increasing number of human diseases, including cancer and autoimmune disorders. Consequently, mTOR inhibitors have found wide applications in treating diverse pathological conditions such as cancer, organ transplantation, restenosis, and rheumatoid arthritis.

Sirolimus, also known as rapamycin, is an immunosuppressant drug used to prevent rejection in organ transplantation; it is especially useful in kidney transplants. Sirolimus-eluting stents were approved in the United States to treat coronary restenosis. Additionally, sirolimus has been demonstrated as an effective inhibitor of tumor growth in various cell lines and animal models. Other limus drugs, such as analogs of sirolimus, have been designed to improve the pharmacokinetic and pharmacodynamic properties of sirolimus. For example, Temsirolimus was approved in the United States and Europe for the treatment of renal cell carcinoma. Everolimus was approved in the U. S. for treatment of advanced breast cancer, pancreatic neuroendocrine tumors, advanced renal cell carcinoma, and subependymal giant cell astrocytoma (SEGA) associated with Tuberous Sclerosis. The mode of action of sirolimus is to bind the cytosolic protein FK-binding protein 12 (FKBP12), and the sirolimus-FKBP12 complex in turn inhibits the mTOR pathway by directly binding to the mTOR Complex 1 (mTORC1).

Albumin-based nanoparticle compositions have been developed as a drug delivery system for delivering substantially water insoluble drugs. See, for example, U.S. Pat. Nos. 5,916,596; 6,506,405; 6,749,868, and 6,537,579, 7,820,788, and 7,923,536. Abraxane®, an albumin stabilized nanoparticle formulation of paclitaxel, was approved in the United States in 2005 and subsequently in various other countries for treating metastatic breast cancer, non-small cell lung cancer and pancreatic cancer.

The disclosures of all publications, patents, patent applications and published patent applications referred to herein are hereby incorporated herein by reference in their entirety.

### BRIEF SUMMARY OF THE INVENTION

The present application provides methods of treating a colon cancer (such as advanced and/or metastatic colon cancer) in an individual, comprising administering to the individual: a) an effective amount of a composition comprising nanoparticles comprising an mTOR inhibitor (such as a limus drug, such as sirolimus or a derivative thereof) and an albumin, b) an effective amount of anti-VEGF antibody (such as bevacizumab), and c) a therapeutically effective FOLFOX regimen (such as FOLFOX4 or a modified FOLFOX6).

In some embodiments, there is provided a method of treating a colon cancer in an individual, comprising administering to the individual: a) an effective amount of a composition comprising nanoparticles comprising an mTOR inhibitor and an albumin, b) an effective amount of anti-VEGF antibody, c) a therapeutically effective FOLFOX regimen. In some embodiments, the colon cancer comprises an mTOR-activation aberration. In some embodiments, the mTOR-activation aberration comprises a PTEN aberration. In some embodiments, the mTOR-activation aberration further comprises a KRAS aberration. In some embodiments, the mTOR-activation aberration further comprises a second aberration, wherein the second aberration is not a PTEN or a KRAS aberration. In some embodiments, the mTOR inhibitor is a limus drug. In some embodiments, the limus drug is rapamycin.

In some embodiments according to any one of the methods described herein, the anti-VEGF antibody is bevacizumab.

In some embodiments according to any one of the methods described herein, the amount of the mTOR inhibitor in the mTOR inhibitor nanoparticle composition is from about 10 mg/m² to about 30 mg/m². In some embodiments, the amount of the mTOR inhibitor in the mTOR inhibitor nanoparticle composition is from about 30 mg/m² to about 45 mg/m². In some embodiments, the amount of the mTOR inhibitor in the mTOR inhibitor nanoparticle composition is from about 45 mg/m² to about 75 mg/m². In some embodiments, the amount of the mTOR inhibitor in the mTOR inhibitor nanoparticle composition is from about 75 mg/m² to about 100 mg/m².

In some embodiments according to any one of the methods described herein, the mTOR inhibitor nanoparticle composition is administered weekly, once every 2 weeks, or once every 3 weeks.

In some embodiments according to any one of the methods described herein, the mTOR inhibitor nanoparticle composition is administered 2 out of every 3 weeks.

In some embodiments according to any one of the methods described herein, the mTOR inhibitor nanoparticle composition is administered 3 out of every 4 weeks.

In some embodiments according to any one of the methods described herein, the average diameter of the nanoparticles in the composition is no greater than about 200 nm.

In some embodiments according to any one of the methods described herein, the weight ratio of the albumin to the mTOR inhibitor in the nanoparticle composition is no greater than about 9:1.

In some embodiments according to any one of the methods described herein, the nanoparticles comprise the mTOR inhibitor associated with the albumin. In some embodiments, the nanoparticles comprise the mTOR inhibitor coated with the albumin.

In some embodiments according to any one of the methods described herein, the mTOR inhibitor nanoparticle composition is administered intravenously, intraarterially, intraperitoneally, intravesicularly, subcutaneously, intrathecally, intrapulmonarily, intramuscularly, intratracheally, intraocularly, transdermally, orally, or by inhalation. In some embodiments, the mTOR inhibitor nanoparticle composition is administered intravenously.

In some embodiments according to any one of the methods described herein, the amount of the anti-VEGF antibody is from about 1 mg/kg to about 20 mg/kg. In some embodiments, the amount of the anti-VEGF antibody is from about 1 mg/kg to about 5 mg/kg. In some embodiments, the amount of the anti-VEGF antibody is from about 5 mg/kg to about 10 mg/kg. In some embodiments, the amount of the anti-VEGF antibody is from about 10 mg/kg to about 15 mg/kg. In some embodiments, the amount of the anti-VEGF antibody is from about 15 mg/kg to about 20 mg/kg.

In some embodiments according to any one of the methods described herein, the anti-VEGF antibody is administered intravenously, intraarterially, intraperitoneally, intravesicularly, subcutaneously, intrathecally, intrapulmonarily, intramuscularly, intratracheally, intraocularly, transdermally, orally, or by inhalation. In some embodiments, the anti-VEGF antibody is administered intravenously. In some embodiments, the amount of the anti-VEGF antibody is about 10 mg/kg, and wherein the anti-VEGF antibody is administered once every two weeks.

In some embodiments according to any one of the methods described herein, the anti-VEGF antibody is administered weekly, once every two weeks, or once every three weeks.

In some embodiments according to any one of the methods described herein, the FOLFOX regimen is FOLFOX4, FOLFOX6, a modified FOLFOX4, or a modified FOLFOX6 regimen. In some embodiments, the FOLFOX regimen is FOLFOX4, and the anti-VEGF antibody is administered intravenously, once every two weeks with an amount of about 10 mg/kg. In some embodiments, the FOLFOX regimen is a modified FOLFOX6, and the anti-VEGF antibody is administered intravenously, once every two weeks with an amount of about 10 mg/kg.

In some embodiments according to any one of the methods described herein, the mTOR inhibitor and the anti-VEGF antibody and/or at least a portion of the FOLFOX regimen are administered sequentially to the individual.

In some embodiments according to any one of the methods described herein, the anti-VEGF antibody and at least a portion of the FOLFOX regimen are administered sequentially to the individual.

In some embodiments according to any one of the methods described herein, the mTOR inhibitor and the anti-VEGF antibody and/or at least a portion of the FOLFOX regimen are administered simultaneously to the individual.

In some embodiments according to any one of the methods described herein, the anti-VEGF antibody and at least a portion of the FOLFOX regimen are administered simultaneously to the individual.

In some embodiments according to any one of the methods described herein, the mTOR inhibitor and the anti-VEGF antibody and/or at least a portion of the FOLFOX regimen are administered concurrently to the individual.

In some embodiments according to any one of the methods described herein, the anti-VEGF antibody and at least a portion of the FOLFOX regimen are administered concurrently to the individual.

In some embodiments according to any one of the methods described herein, the individual is human.

In some embodiments according to any one of the methods described herein, the method further comprises selecting the individual for treatment based on the presence of at least one mTOR-activation aberration or the MSI status. In some embodiments, the mTOR-activating aberration comprises a mutation in an mTOR-associated gene. In some embodiments, the mTOR-activating aberration is in at least one mTOR-associated gene selected from the group consisting of AKT1, FLT-3, MTOR, PIK3CA, PIK3CG, TSC1, TSC2, RHEB, STK11, NF1, NF2, TP53, FGFR4, BAP1, KRAS, NRAS and PTEN. In some embodiments, the mTOR-activating aberration is in PTEN.

In some embodiments according to any one of the methods described herein, the method further comprises assessing an mTOR-activating aberration in the individual. In some embodiments, the mTOR-activating aberration is assessed by gene sequencing or immunohistochemistry.

In some embodiments according to any one of the methods described herein, the method further comprises selecting the individual for treatment based on at least one biomarker indicative of favorable response to treatment with an anti-VEGF antibody.

In some embodiments according to any one of the methods described herein, the method further comprises selecting the individual for treatment based on at least one biomarker indicative of favorable response to treatment with FOLFOX.

In some embodiments according to any one of the methods described herein, the colon cancer is advanced, malignant, and/or metastatic.

In some embodiments according to any one of the methods described herein, the colon cancer is stage I, II, III, or IV cancer.

In some embodiments according to any one of the methods described herein, the colon cancer is characterized with a genomic instability. In some embodiments, the genomic instability comprises a microsatellite instability (MSI), a chromosomal instability (CIN) and/or a CpG island methylator phenotype (CIMP).

In some embodiments according to any one of the methods described herein, the colon cancer is characterized with an alteration of a pathway, wherein the alteration of a pathway comprises PTEN, TP53, BRAF, PI3CA or APC gene inactivation, KRAS, TGF-β, CTNNB, Epithelial-to-mesenchymal transition (EMT) genes or WNT-signaling activation, and/or MYC amplification.

In some embodiments according to any one of the methods described herein, the colon cancer is classified under the colon cancer subtype (CCS) system as CCS1, CCS2, or CCS3.

In some embodiments according to any one of the methods described herein, the colon cancer is classified under colorectal cancer assigner (CRCA system) as stem-like, goblet-like, inflammatory, transit-amplifying, or enterocyte subtype.

In some embodiments according to any one of the methods described herein, the individual has been previously treated with chemotherapy, radiation or surgery.

In some embodiments according to any one of the methods described herein, the individual has not been previously treated.

In some embodiments according to any one of the methods described herein, the method is used as an adjuvant treatment.

### DETAILED DESCRIPTION OF THE INVENTION

The present application provides methods of combination therapy for treating a colon cancer in an individual, comprising administering to the individual an effective amount of a composition comprising nanoparticles comprising an mTOR inhibitor (e.g., a limus drug, e.g., sirolimus or a derivative thereof) and an albumin in conjunction with an effective amount of anti-VEGF antibody and a therapeutically effective FOLFOX regimen.

### Definitions

As used herein “Nab”® stands for nanoparticle albumin-bound, and “nab-sirolimus” is an albumin stabilized nanoparticle formulation of sirolimus. Nab-sirolimus is also known as nab-rapamycin, which has been previously described. See, for example, WO2008109163A1, WO2014151853, WO2008137148A2, and WO2012149451A1, each of which is incorporated herein by reference in their entirety.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, reducing recurrence rate of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. In some embodiments, the treatment reduces the severity of one or more symptoms associated with cancer by at least about any of 10%, 20%, 30%, 40%, 50%, 60%, 70%, 80%, 90%, 95% or 100% compared to the corresponding symptom in the same subject prior to treatment or compared to the corresponding symptom in other subjects not receiving the treatment. Also encompassed by “treatment” is a reduction of pathological consequence of cancer. The methods of the invention contemplate any one or more of these aspects of treatment.

The terms “recurrence,” “relapse” or “relapsed” refers to the return of a cancer or disease after clinical assessment of the disappearance of disease. A diagnosis of distant metastasis or local recurrence can be considered a relapse.

The term “refractory” or “resistant” refers to a cancer or disease that has not responded to treatment.

As used herein, an “at risk” individual is an individual who is at risk of developing cancer. An individual “at risk” may or may not have detectable disease, and may or may not have displayed detectable disease prior to the treatment methods described herein. “At risk” denotes that an individual has one or more so-called risk factors, which are measurable parameters that correlate with development of cancer, which are described herein. An individual having one or more of these risk factors has a higher probability of developing cancer than an individual without these risk factor(s).

“Adjuvant setting” refers to a clinical setting in which an individual has had a history of cancer, and generally (but not necessarily) been responsive to therapy, which includes, but is not limited to, surgery (e.g., surgery resection), radiotherapy, and chemotherapy. However, because of their history of cancer, these individuals are considered at risk of development of the disease. Treatment or administration in the “adjuvant setting” refers to a subsequent mode of treatment. The degree of risk (e.g., when an individual in the adjuvant setting is considered as “high risk” or “low risk”) depends upon several factors, most usually the extent of disease when first treated.

“Neoadjuvant setting” refers to a clinical setting in which the method is carried out before the primary/definitive therapy.

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of subjects. Cancer development can be detectable using standard methods, including, but not limited to, computerized axial tomography (CAT scan), Magnetic Resonance Imaging (MRI), ultrasound, clotting tests, arteriography, biopsy, urine cytology, and cystoscopy. Development may also refer to cancer progression that may be initially undetectable and includes occurrence, recurrence, and onset.

The term “effective amount” used herein refers to an amount of a compound or composition sufficient to treat a specified disorder, condition or disease such as ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other unwanted cell proliferation in cancer. In some embodiments, an effective amount is an amount sufficient to delay development of cancer. In some embodiments, an effective amount is an amount sufficient to prevent or delay recurrence. In some embodiments, an effective amount is an amount sufficient to reduce recurrence rate in the individual. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) reduce recurrence rate of tumor, and/or (viii) relieve to some extent one or more of the symptoms associated with the cancer.

As is understood in the art, an “effective amount” may be in one or more doses, i.e., a single dose or multiple doses may be required to achieve the desired treatment endpoint. An effective amount may be considered in the context of administering one or more therapeutic agents, and a nanoparticle composition (e.g., a composition including sirolimus and an albumin) may be considered to be given in an effective amount if, in conjunction with one or more other agents, a desirable or beneficial result may be or is achieved. The components (e.g., the first and second therapies) in a combination therapy of the invention may be administered sequentially, simultaneously, or concurrently using the same or different routes of administration for each component. Thus, an effective amount of a combination therapy includes an amount of the first therapy and an amount of the second therapy that when administered sequentially, simultaneously, or concurrently produces a desired outcome.

“In conjunction with” or “in combination with” refers to administration of one treatment modality in addition to another treatment modality, such as administration of a nanoparticle composition described herein in addition to administration of the other agent to the same individual under the same treatment plan. As such, “in conjunction with” or “in combination with” refers to administration of one treatment modality before, during or after delivery of the other treatment modality to the individual.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy is contained in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, “specific”, “specificity”, or “selective” or “selectivity” as used when describing a compound as an inhibitor, means that the compound preferably interacts with (e.g., binds to, modulates, and inhibits) a particular target (e.g., a protein and an enzyme) than a non-target. For example, the compound has a higher affinity, a higher avidity, a higher binding coefficient, or a lower dissociation coefficient for a particular target. The specificity or selectivity of a compound for a particular target can be measured, determined, or assessed by using various methods well known in the art. For example, the specificity or selectivity can be measured, determined, or assessed by measuring the IC₅₀ of a compound for a target. A compound is specific or selective for a target when the IC₅₀ of the compound for the target is 2-fold, 4-fold, 6-fold, 8-fold, 10-fold, 20-fold, 50-fold, 100-fold, 500-fold, 1000-fold, or more than the IC₅₀ of the same compound for a non-target. IC₅₀ can be determined by commonly known methods in the art.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U. S. Food and Drug administration.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

As used herein and in the appended claims, the singular forms “a,” “or,” and “the” include plural referents unless the context clearly dictates otherwise.

As used herein, the terms “colorectal cancer” and “colon cancer” are used interchangeably herein to refer to any cancerous neoplasia of the colon (including the rectum).

As used herein, the term “genomic instability” is defined to include a broad class of disruptions in genomic nucleotide sequences. Such disruptions include the loss of heterozygosity (usually characterized by massive loss of chromosomal DNA), microsatellite instability (usually indicative of defects in DNA repair mechanisms), and mutations (which include insertions, deletions, substitutions, duplications, rearrangements, or modifications).

### Methods of Treating a Colon Cancer

The present application provides a variety of methods of using nanoparticle compositions with an mTOR inhibitor (e.g., rapamycin) and a carrier protein (e.g., albumin) in combination with an anti-VEGF antibody and a FOLFOX regimen to treat a colon cancer, such as advanced colon cancer, malignant colon cancer, metastatic colon cancer, stage I, II, III, or IV colon cancer, a colon cancer characterized with a genomic instability, a colon cancer characterized with an alteration of a pathway, a colon cancer classified under the colon cancer subtype (CCS) system as CCS1, CCS2, or CCS3, a colon cancer classified under colorectal cancer assigner (CRCA system) as stem-like, goblet-like, inflammatory, transit-amplifying, or enterocyte subtype, a colon cancer classified under the colon cancer molecular subtype (CCMS) system as C1, C2, C3, C4, C5, or C6 subtype, a colon cancer classified under the CRC intrinsic subtype (CRCIS) system as Type A, Type B, or Type C subtype, or a colon cancer classified under the colorectal cancer subtyping consortium (CRCSC) classification system as CMS1, CMS2, CMS3, or CMS4. In some embodiments, the colon cancer has a microsatellite instability (MSI) status of MSI-high or MSI-low. In some embodiments, the colon cancer is characterized with a mutation in KRAS, NRAS and/or BRAF. In some embodiments, the individual has previously undergone a therapy (e.g., chemotherapy, radiation, surgery or immunomodulatory therapy). In some embodiments, the individual does not respond to a previous therapy (e.g., chemotherapy, radiation, surgery or immunomodulatory therapy).

In some embodiments, there is provided a method of treating colon cancer (e.g., a metastatic colon cancer) in an individual, comprising administering to the individual: a) an effective amount of a composition comprising nanoparticles comprising an mTOR inhibitor and an albumin, b) an effective amount of anti-VEGF antibody (e.g., bevacizumab), c) a therapeutically effective FOLFOX regimen. In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the amount of the mTOR inhibitor in the mTOR inhibitor nanoparticle composition is selected from the group consisting of about 10 mg/m² to about 30 mg/m², about 30 mg/m² to about 45 mg/m², about 45 mg/m² to about 75 mg/m² and about 45 mg/m² to about 75 mg/m². In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the amount of the anti-VEGF antibody is from about 5 mg/kg to about 10 mg/kg. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising an mTOR inhibitor and an albumin, b) an effective amount of anti-VEGF antibody (e.g., bevacizumab), c) a therapeutically effective FOLFOX regimen, wherein the FOLFOX regimen comprises administrating oxaliplatin, leucovorin and 5-fluoruracil (5-FU) into the individual. In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the amount of the mTOR inhibitor in the mTOR inhibitor nanoparticle composition is selected from the group consisting of about 10 mg/m² to about 30 mg/m², about 30 mg/m² to about 45 mg/m², about 45 mg/m² to about 75 mg/m² and about 45 mg/m² to about 75 mg/m². In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the amount of the anti-VEGF antibody is from about 5 mg/kg to about 10 mg/kg. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising an mTOR inhibitor and an albumin, b) an effective amount of anti-VEGF antibody (e.g., bevacizumab), c) a therapeutically effective FOLFOX regimen, wherein the FOLFOX regimen comprises i) administering oxaliplatin in an amount of from about 50 mg/m² to about 200 mg/m²; ii) administering leucovorin in the amount of from about 200 mg/m² to about 600 mg/m²; iii) administering 5-fluoruracil (5-FU) in the amount of from about 1200 mg/m² to about 3600 mg/m². In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the amount of the mTOR inhibitor in the mTOR inhibitor nanoparticle composition is selected from the group consisting of about 10 mg/m² to about 30 mg/m², about 30 mg/m² to about 45 mg/m², about 45 mg/m² to about 75 mg/m² and about 45 mg/m² to about 75 mg/m². In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the amount of the anti-VEGF antibody is from about 5 mg/kg to about 10 mg/kg. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising an mTOR inhibitor and an albumin, b) an effective amount of anti-VEGF antibody (e.g., bevacizumab), c) a therapeutically effective FOLFOX regimen, wherein the FOLFOX regimen is FOLFOX4. In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the amount of the mTOR inhibitor in the mTOR inhibitor nanoparticle composition is selected from the group consisting of about 10 mg/m² to about 30 mg/m², about 30 mg/m² to about 45 mg/m², about 45 mg/m² to about 75 mg/m² and about 45 mg/m² to about 75 mg/m². In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the amount of the anti-VEGF antibody is from about 5 mg/kg to about 10 mg/kg. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising an mTOR inhibitor and an albumin, b) an effective amount of anti-VEGF antibody (e.g., bevacizumab), c) a therapeutically effective FOLFOX regimen, wherein the FOLFOX regimen is FOLFOX6 or a modified FOLFOX6. In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the amount of the mTOR inhibitor in the mTOR inhibitor nanoparticle composition is selected from the group consisting of about 10 mg/m² to about 30 mg/m², about 30 mg/m² to about 45 mg/m², about 45 mg/m² to about 75 mg/m² and about 45 mg/m² to about 75 mg/m². In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the amount of the anti-VEGF antibody is from about 5 mg/kg to about 10 mg/kg. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising a limus drug (such as rapamycin or its derivative) and an albumin, b) an effective amount of anti-VEGF antibody (e.g., bevacizumab), c) a therapeutically effective FOLFOX regimen. In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the amount of the mTOR inhibitor in the mTOR inhibitor nanoparticle composition is selected from the group consisting of about 10 mg/m² to about 30 mg/m², about 30 mg/m² to about 45 mg/m², about 45 mg/m² to about 75 mg/m² and about 45 mg/m² to about 75 mg/m². In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the amount of the anti-VEGF antibody is from about 5 mg/kg to about 10 mg/kg. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising a limus drug (such as rapamycin or its derivative) and an albumin, b) an effective amount of anti-VEGF antibody (e.g., bevacizumab), c) a therapeutically effective FOLFOX regimen, wherein the FOLFOX regimen comprises administrating oxaliplatin, leucovorin and 5-fluoruracil (5-FU) into the individual. In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the amount of the mTOR inhibitor in the mTOR inhibitor nanoparticle composition is selected from the group consisting of about 10 mg/m² to about 30 mg/m², about 30 mg/m² to about 45 mg/m², about 45 mg/m² to about 75 mg/m² and about 45 mg/m² to about 75 mg/m². In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the amount of the anti-VEGF antibody is from about 5 mg/kg to about 10 mg/kg. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising a limus drug (such as rapamycin or its derivative) and an albumin, b) an effective amount of anti-VEGF antibody (e.g., bevacizumab), c) a therapeutically effective FOLFOX regimen, wherein the FOLFOX regimen comprises i) administering oxaliplatin in an amount of from about 50 mg/m² to about 200 mg/m²; ii) administering leucovorin in the amount of from about 200 mg/m² to about 600 mg/m²; iii) administering 5-fluoruracil (5-FU) in the amount of from about 1200 mg/m² to about 3600 mg/m². In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the amount of the mTOR inhibitor in the mTOR inhibitor nanoparticle composition is selected from the group consisting of about 10 mg/m² to about 30 mg/m², about 30 mg/m² to about 45 mg/m², about 45 mg/m² to about 75 mg/m² and about 45 mg/m² to about 75 mg/m². In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the amount of the anti-VEGF antibody is from about 5 mg/kg to about 10 mg/kg. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising a limus drug (such as rapamycin or its derivative) and an albumin, b) an effective amount of anti-VEGF antibody (e.g., bevacizumab), c) a therapeutically effective FOLFOX regimen, wherein the FOLFOX regimen is FOLFOX4. In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the amount of the mTOR inhibitor in the mTOR inhibitor nanoparticle composition is selected from the group consisting of about 10 mg/m² to about 30 mg/m², about 30 mg/m² to about 45 mg/m², about 45 mg/m² to about 75 mg/m² and about 45 mg/m² to about 75 mg/m². In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the amount of the anti-VEGF antibody is from about 5 mg/kg to about 10 mg/kg. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising a limus drug (such as rapamycin or its derivative) and an albumin, b) an effective amount of anti-VEGF antibody (e.g., bevacizumab), c) a therapeutically effective FOLFOX regimen, wherein the FOLFOX regimen is FOLFOX6 or a modified FOLFOX6. In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the amount of the mTOR inhibitor in the mTOR inhibitor nanoparticle composition is selected from the group consisting of about 10 mg/m² to about 30 mg/m², about 30 mg/m² to about 45 mg/m², about 45 mg/m² to about 75 mg/m² and about 45 mg/m² to about 75 mg/m². In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the amount of the anti-VEGF antibody is from about 5 mg/kg to about 10 mg/kg. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising sirolimus (i.e., rapamycin) and an albumin, b) an effective amount of anti-VEGF antibody (e.g., bevacizumab), c) a therapeutically effective FOLFOX regimen. In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the amount of the mTOR inhibitor in the mTOR inhibitor nanoparticle composition is selected from the group consisting of about 10 mg/m² to about 30 mg/m², about 30 mg/m² to about 45 mg/m², about 45 mg/m² to about 75 mg/m² and about 45 mg/m² to about 75 mg/m². In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the amount of the anti-VEGF antibody is from about 5 mg/kg to about 10 mg/kg. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising sirolimus (i.e., rapamycin) and an albumin, b) an effective amount of anti-VEGF antibody (e.g., bevacizumab), c) a therapeutically effective FOLFOX regimen, wherein the FOLFOX regimen comprises administrating oxaliplatin, leucovorin and 5-fluoruracil (5-FU) into the individual. In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the amount of the mTOR inhibitor in the mTOR inhibitor nanoparticle composition is selected from the group consisting of about 10 mg/m² to about 30 mg/m², about 30 mg/m² to about 45 mg/m², about 45 mg/m² to about 75 mg/m² and about 45 mg/m² to about 75 mg/m². In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the amount of the anti-VEGF antibody is from about 5 mg/kg to about 10 mg/kg. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising an mTOR inhibitor and an albumin, wherein the mTOR inhibitor is sirolimus (i.e., rapamycin) or a derivative thereof, b) an effective amount of anti-VEGF antibody (e.g., bevacizumab), c) a therapeutically effective FOLFOX regimen, wherein the FOLFOX regimen comprises i) administering oxaliplatin in an amount of from about 50 mg/m² to about 200 mg/m²; ii) administering leucovorin in the amount of from about 200 mg/m² to about 600 mg/m²; iii) administering 5-fluoruracil (5-FU) in the amount of from about 1200 mg/m² to about 3600 mg/m². In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the amount of the mTOR inhibitor in the mTOR inhibitor nanoparticle composition is selected from the group consisting of about 10 mg/m² to about 30 mg/m², about 30 mg/m² to about 45 mg/m², about 45 mg/m² to about 75 mg/m² and about 45 mg/m² to about 75 mg/m². In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the amount of the anti-VEGF antibody is from about 5 mg/kg to about 10 mg/kg. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising an mTOR inhibitor and an albumin, wherein the mTOR inhibitor is sirolimus (i.e., rapamycin) or a derivative thereof, b) an effective amount of anti-VEGF antibody (e.g., bevacizumab), c) a therapeutically effective FOLFOX regimen, wherein the FOLFOX regimen is FOLFOX4. In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the amount of the mTOR inhibitor in the mTOR inhibitor nanoparticle composition is selected from the group consisting of about 10 mg/m² to about 30 mg/m², about 30 mg/m² to about 45 mg/m², about 45 mg/m² to about 75 mg/m² and about 45 mg/m² to about 75 mg/m². In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the amount of the anti-VEGF antibody is from about 5 mg/kg to about 10 mg/kg. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising an mTOR inhibitor and an albumin, wherein the mTOR inhibitor is sirolimus (i.e., rapamycin) or a derivative thereof, b) an effective amount of anti-VEGF antibody (e.g., bevacizumab), c) a therapeutically effective FOLFOX regimen, wherein the FOLFOX regimen is FOLFOX6 or a modified FOLFOX6. In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the amount of the mTOR inhibitor in the mTOR inhibitor nanoparticle composition is selected from the group consisting of about 10 mg/m² to about 30 mg/m², about 30 mg/m² to about 45 mg/m², about 45 mg/m² to about 75 mg/m² and about 45 mg/m² to about 75 mg/m². In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the amount of the anti-VEGF antibody is from about 5 mg/kg to about 10 mg/kg. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising an mTOR inhibitor (e.g., a limus drug, e.g., sirolimus or a derivative thereof) and an albumin, b) an effective amount of anti-VEGF antibody (e.g., bevacizumab), c) a therapeutically effective FOLFOX regimen, wherein the individual comprises an mTOR-activation aberration in PTEN. In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the amount of the mTOR inhibitor in the mTOR inhibitor nanoparticle composition is selected from the group consisting of about 10 mg/m² to about 30 mg/m², about 30 mg/m² to about 45 mg/m², about 45 mg/m² to about 75 mg/m² and about 45 mg/m² to about 75 mg/m². In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the amount of the anti-VEGF antibody is from about 5 mg/kg to about 10 mg/kg. In some embodiments, the anti-VEGF antibody is administered intravenously. In some embodiments, the anti-VEGF antibody is administered weekly, once every two weeks, or once every three weeks. In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising an mTOR inhibitor (e.g., a limus drug, e.g., sirolimus or a derivative thereof) and an albumin, b) an effective amount of anti-VEGF antibody (e.g., bevacizumab), c) a therapeutically effective FOLFOX regimen, wherein the individual comprises a first mTOR-activation aberration in PTEN and a second mTOR-activation aberration in KRAS. In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the amount of the mTOR inhibitor in the mTOR inhibitor nanoparticle composition is selected from the group consisting of about 10 mg/m² to about 30 mg/m², about 30 mg/m² to about 45 mg/m², about 45 mg/m² to about 75 mg/m² and about 45 mg/m² to about 75 mg/m². In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the amount of the anti-VEGF antibody is from about 5 mg/kg to about 10 mg/kg. In some embodiments, the anti-VEGF antibody is administered intravenously. In some embodiments, the anti-VEGF antibody is administered weekly, once every two weeks, or once every three weeks. In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising an mTOR inhibitor (e.g., a limus drug, e.g., sirolimus or a derivative thereof) and an albumin, b) an effective amount of anti-VEGF antibody (e.g., bevacizumab), c) a therapeutically effective FOLFOX regimen, wherein the individual comprises a first mTOR-activation aberration in PTEN and a second mTOR-activation aberration, wherein the second aberration is not a PTEN or KRAS aberration. In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the amount of the mTOR inhibitor in the mTOR inhibitor nanoparticle composition is selected from the group consisting of about 10 mg/m² to about 30 mg/m², about 30 mg/m² to about 45 mg/m², about 45 mg/m² to about 75 mg/m² and about 45 mg/m² to about 75 mg/m². In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the amount of the anti-VEGF antibody is from about 5 mg/kg to about 10 mg/kg. In some embodiments, the anti-VEGF antibody is administered intravenously. In some embodiments, the anti-VEGF antibody is administered weekly, once every two weeks, or once every three weeks. In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising sirolimus and an albumin, b) an effective amount of anti-VEGF antibody (e.g., bevacizumab), c) a therapeutically effective FOLFOX regimen, wherein the amount of anti-VEGF antibody is about 10 mg/kg, and wherein the FOLFOX regimen is a modified FOLFOX6 regimen. In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the amount of the mTOR inhibitor in the mTOR inhibitor nanoparticle composition is selected from the group consisting of about 10 mg/m² to about 30 mg/m², about 30 mg/m² to about 45 mg/m², about 45 mg/m² to about 75 mg/m² and about 45 mg/m² to about 75 mg/m². In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising sirolimus and an albumin, b) an effective amount of anti-VEGF antibody (e.g., bevacizumab), c) a therapeutically effective FOLFOX regimen, wherein the amount of sirolimus in the mTOR inhibitor nanoparticle composition is from 10 mg/m² to about 60 mg/m², wherein the amount of anti-VEGF antibody is about 10 mg/kg, and wherein the FOLFOX regimen is a modified FOLFOX6 regimen. In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising sirolimus and an albumin, b) an effective amount of anti-VEGF antibody (e.g., bevacizumab), c) a therapeutically effective FOLFOX regimen, wherein the amount of anti-VEGF antibody is about 10 mg/kg, and wherein the FOLFOX regimen is a modified FOLFOX6 regimen comprising i) administering oxaliplatin in an amount of about 85 mg/m²; ii) administering leucovorin in the amount of from about 400 mg/m²; iii) administering 5-fluoruracil (5-FU) in the amount of from about 2800 mg/m². In some embodiments, the mTOR inhibitor and the anti-VEGF antibody and/or at least a portion of the FOLFOX regimen are administered sequentially to the individual. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising sirolimus and an albumin, b) an effective amount of anti-VEGF antibody (e.g., bevacizumab), c) a therapeutically effective FOLFOX regimen, wherein the amount of sirolimus in the mTOR inhibitor nanoparticle composition is from 10 mg/m² to about 60 mg/m², wherein the amount of anti-VEGF antibody is about 10 mg/kg, and wherein the FOLFOX regimen is a modified FOLFOX6 regimen comprising i) administering oxaliplatin in an amount of about 85 mg/m²; ii) administering leucovorin in the amount of about 400 mg/m²; iii) administering 5-fluoruracil (5-FU) in the amount of about 2800 mg/m². In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising sirolimus and an albumin, b) an effective amount of anti-VEGF antibody (e.g., bevacizumab), c) a therapeutically effective FOLFOX regimen, wherein the nanoparticle composition comprising sirolimus, the anti-VEGF antibody and the FOLFOX regimen is administered according to a regimen in Table 2. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g. a metastatic colon cancer) without weight loss in an individual, comprising administering to the individual: a) an effective amount of a composition comprising nanoparticles comprising an mTOR inhibitor and an albumin, b) an effective amount of anti-VEGF antibody (e.g., bevacizumab), c) a therapeutically effective FOLFOX regimen. In some embodiments, the FOLFOX regimen comprises administrating oxaliplatin, leucovorin and 5-fluoruracil (5-FU) into the individual. In some embodiments, there is provided a method of treating a colon cancer (e.g., a metastatic colon cancer) without weight loss in an individual, comprising administering to the individual: a) an effective amount of a composition comprising nanoparticles comprising sirolimus and an albumin, b) an effective amount of anti-VEGF antibody (e.g., bevacizumab), c) a therapeutically effective FOLFOX regimen, wherein the nanoparticle composition comprising sirolimus, the anti-VEGF antibody and the FOLFOX regimen is administered according to a regimen in Table 2. In some embodiments, the colon cancer has metastasized to one, two, three, or more other organs (e.g., pancreas, liver, lung, kidney, bone, brain). In some embodiments, the cancer in other organs metastasized from the colon cancer shrinked (e.g., by at least 5%, 10%, 15%, 20%, 25%, 30%, or more) following treatment. In some embodiments, the individual has a weight within 95%, 96%, or 97% of the weight right before the treatment shortly after the treatment (for example, within about six months, five months, four months, three and a half months, three months, two and a half months, or two months after treatment). In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, there is provided a method of treating colon cancer (e.g., a metastatic colon cancer) in an individual, comprising administering to the individual: a) an effective amount of a composition comprising nanoparticles comprising an mTOR inhibitor and an albumin, b) an effective amount of anti-VEGF antibody (e.g., bevacizumab), c) a therapeutically effective FOLFOX regimen, wherein the individual gains weight after being treated. In some embodiments, there is provided a method of treating colon cancer (e.g., a metastatic colon cancer) in an individual, comprising administering to the individual: a) an effective amount of a composition comprising nanoparticles comprising sirolimus and an albumin, b) an effective amount of anti-VEGF antibody (e.g., bevacizumab), c) a therapeutically effective FOLFOX regimen, wherein the nanoparticle composition comprising sirolimus, the anti-VEGF antibody and the FOLFOX regimen is administered according to a regimen in Table 2, wherein the individual gains weight after being treated. In some embodiments, the colon cancer has metastasized to one, two, three, or more other organs (e.g., pancreas, liver, lung, kidney, bone, brain). In some embodiments, the cancer in other organs metastasized from the colon cancer shrinked (e.g., by at least 5%, 10%, 15%, 20%, 25%, 30%, or more) following treatment. In some embodiments, the individual gains at least 1%, 2%, 3%, 4%, 5%, 6%, 7%, 8%, 9%, 10%, 11%, 12% or more weight within about six months, five months, four months, three and a half months, three months, two and a half months, or two months after treatment. In some embodiments, the anti-VEGF antibody and at least a portion of the FOLFOX regimen are administered sequentially to the individual. In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. In some embodiments, the mTOR inhibitor and the anti-VEGF antibody and/or at least a portion of the FOLFOX regimen are administered concurrently to the individual. In some embodiments, the anti-VEGF antibody and at least a portion of the FOLFOX regimen are administered concurrently to the individual. In some embodiments, the mTOR inhibitor nanoparticle composition is administered weekly, every other week, 2 out of every 3 weeks, or 3 out of every 4 weeks. In some embodiments, the mTOR inhibitor nanoparticle composition is administered intravenously. In some embodiments, the anti-VEGF antibody is administered intravenously. In some embodiments, the anti-VEGF antibody is administered weekly, once every two weeks, or once every three weeks. In some embodiments, the individual is human. In some embodiments, the individual has at least one mTOR activation aberration (e.g., a mutation in PTEN). In some embodiments, the method further comprising selecting the individual for treatment based on the presence of at least one mTOR-activation aberration. In some embodiments, the mTOR-activating aberration comprises a mutation in PTEN.

In some embodiments, a tumor biomarker decreases after treatment. In some embodiments, the tumor biomarker is carcinoembryonic antigen (CEA). In some embodiments, the CEA level decreases by at least about 1-fold, two-fold, or three-fold.

In some embodiments, the colon cancer has metastasized to one, two, three, or more other organs (e.g., pancreas, liver, lung, kidney, bone, brain). In some embodiments, the cancer in another organ that is metastasized from the colon cancer shrinked (e.g., by at least 5%, 10%, 15%, 20%, 25%, 30%, or more) following treatment. In some embodiments, the shrinkage is present after at least about one week, two weeks, three weeks or fours weeks after treatment. In some embodiments, the shrinkage is present after at least about one month, one and a half months, two months, two and a half months, or three months after treatment. In some embodiments, the colon cancer or the cancer in another organ that is metastasized from the colon cancer has significant necrosis following treatment. In some embodiments, the significant necrosis is present after at least about one week, two weeks, three weeks or fours weeks after treatment. In some embodiments, the significant necrosis is present after at least about one month, one and a half months, two months, two and a half months, or three months after treatment. In some embodiments, an adjacent lymph node close to the colon cancer or the cancer in another organ that is metastasized from the colon cancer has a decrease in size after treatment. In some embodiments, the decrease in size is present after at least about one week, two weeks, three weeks or fours weeks after treatment. In some embodiments, the decrease in size is present after at least about one month, one and a half months, two months, two and a half months, or three months after treatment.

In some embodiments, the individual does not exhibit a severe toxicity following treatment. In some embodiments, the severe toxicity is severe cytokine release syndrome (CRS), optionally grade 3 or higher, prolonged grade 3 or higher or grade 4 or 5 CRS. In some embodiments, the individual does not have a substantial increase (for example, less than 5%, 10%, 15%, 20%, 25%, or 30%) in cytokine (such as IFN-gamma, TNF-alpha) after treatment.

### Pharmaceutical Compositions

The nanoparticle compositions (such as mTOR inhibitor nanoparticle compositions) and/or an anti-VEGF antibody and/or a portion of (or a component of) FOLFOX regimen described herein can be used in the preparation of a formulation, such as a pharmaceutical composition, by combining the nanoparticle composition(s) and an anti-VEGF antibody and/or a portion of (or a component of) FOLFOX regimen described herein with a pharmaceutically acceptable carrier, an excipient, a stabilizing agent, and/or another agent known in the art for use in the methods of treatment, methods of administration, and dosage regimes described herein. In some embodiments, one or some components described herein (e.g., the nanoparticle composition(s), an anti-VEGF antibody or a portion of (or a component of) FOLFOX regimen) can be provided in a single composition.

### Colon Cancer to be Treated

In some embodiments, there is provided a method of treating a colon cancer in an individual (such as human), comprising administering to the individual a) an effective amount of a composition comprising nanoparticles comprising an mTOR inhibitor (such as a limus drug, e.g., sirolimus (i.e., rapamycin) or a derivative thereof) and an albumin, b) an effective amount of anti-VEGF antibody (e.g., Avastin) and c) a therapeutically effective FOLFOX regimen. In some embodiments, the method is used to treat a primary tumor. In some embodiments, the method is used to treat metastatic cancer (that is, cancer that has metastasized from the primary tumor) is provided. In some embodiments, the method is used to treat a tumor of low malignant potential (e.g., a borderline tumor), such as an early or late stage tumor of low malignant potential. In some embodiments, there is provided a method of treating colon cancer at an advanced stage. In some embodiments, the method is for the treatment of an early stage colon cancer.

The methods may be practiced in an adjuvant setting. The methods provided herein may also be practiced in a neoadjuvant setting, i.e., the method may be carried out before the primary/definitive therapy. In some embodiments, the individual has previously been treated. In some embodiments, the individual has not previously been treated. In some embodiments, the treatment is a first line therapy. In some embodiments, the treatment is a second line therapy. In some embodiments, the treatment is a third line therapy. In some embodiments, the individual is at risk of developing colon cancer but has not been diagnosed with colon cancer. In some embodiments, the colon cancer has reoccurred after a remission.

In various embodiments, the method described herein is used to treat colon cancer at different stages. In some embodiments, the method is used to treat stage I colon cancer. In some embodiments, the method is used to treat stage II (for example, stage IIA, IIB, or IIC) colon cancer. In some embodiments, the method is used to treat stage III (for example, stage IIIA, IIIB, or IIIC) colon cancer. In some embodiments, the method is used to treat stage IV (for example, stage IVA, IVB, or IVC) colon cancer. In some embodiments, the method is used to treat stage 0 colon cancer (i.e., carcinoma in situ).

In some embodiments, the colon cancer is characterized with a genomic instability. In some embodiments, the genomic instability comprises at least one modification of genomic DNA. In some embodiments, the modification is a chromosomal instability (CIN). In some embodiments, the modification is a loss of heterozygosity (e.g., a massive loss of chromosomal DNA). In some embodiments, the modification is a microsatellite instability (MSI). In some embodiments, the modification is a mutation (e.g., an insertion, a deletion, a substitution, a duplication, a rearrangement) in the nucleotide sequence. In some embodiments, the modification of genomic DNA comprises a modification of DNA methylation or histone modification. In some embodiments, the colon cancer is characterized with at least 5%, 6%, 7%, 8%, 9%, 10%, 11%, 12%, 13%, 14%, 15%, 16%, 17%, or 18% lower total DNA methylation than normal tissue. In some embodiments, the modification of genomic DNA comprises a CpG island methylator phenotype (CIMP). In some embodiments, the colon cancer is characterized with a modified CpG island methylation. In some embodiments the modified CpG island methylation comprises hypermethylation of a CpG-rich promoter.

In some embodiments, the colon cancer is characterized with an alteration of a pathway. In some embodiments, the alteration of a pathway comprises TP53, BRAF, PI3CA or APC gene inactivation, KRAS, TGF-β, CTNNB, Epithelial-to-mesenchymal transition (EMT) genes or WNT-signaling activation, and/or MYC or CDK8 amplification. In some embodiments, the colon cancer is characterized with an alteration of a KRAS mutation or a BRAF mutation. In some embodiments, the alteration of a pathway is assessed/detected by genomic sequencing. In some embodiments, the alteration of a pathway is detected by assessing the expression (e.g., mRNA or protein expression) of a gene in the cancer tissue. In some embodiments, the pathway is selected from the group consisting of WNT, MAPK, PI3K, TGF-β and p53 pathways. In some embodiments, the colon cancer is characterized with the alterations of at least two, three, four or five pathway as discussed above.

In various embodiments, the colon cancer can be classified under different system as different subtype. Some examples of classification systems are described in for example, Rodriguez-Salas et al., Crit Rev Oncol Hematol. 2017 January; 109:9-19; De Sousa E Melo et al., Nat Med. 2013 May; 19(5):614-8; Sadanandam et al., Nat Med. 2013 May; 19(5):619-25; Marisa et al., PloS Med. 2013; 10(5); Roepman et al., Int J Cancer. 2014 Feb. 1; 134(3):552-62; Salazar et al., J Clin Oncol. 2011 Jan. 1; 29(1):17-24.

I. Colon Cancer Subtype (CCS) System

In some embodiments, the colon cancer is classified under the colon cancer subtype (CCS) system as CCS1. In some embodiments, the colon cancer further comprises a mutation in KRAS or TP53. In some embodiments, the colon cancer is further characterized with a CIN (e.g., a loss of heterozygosity). In some embodiments, the colon cancer is further characterized with a higher activity of the WNT signaling cascade compared to a normal tissue. In some embodiments, the colon cancer is resistant to a therapy comprising an anti-EGFR antibody (e.g., cetuximab).

In some embodiments, the colon cancer is classified under the colon cancer subtype (CCS) system as CCS2. In some embodiments, the colon cancer is characterized with tumors for MSI or CpG island methylator phenotype (CIMP). In some embodiments, the colon cancer is characterized with an inflammatory cell infiltration. In some embodiments, the inflammatory cell infiltration is located in the right colon. In some embodiments, the colon cancer is resistant to a therapy comprising an anti-EGFR antibody (e.g., cetuximab).

In some embodiments, the colon cancer is classified under the colon cancer subtype (CCS) system as CCS3. In some embodiments, the colon cancer is characterized with a genomic instability comprising a MSI or a CIN. In some embodiments, the colon cancer is characterized with a higher expression of genes related to Epithelial-to-mesenchymal transition (EMT), matrix remodeling and cell migration. In some embodiments, the colon cancer is characterized with an activated TGF-β pathway. In some embodiments, the colon cancer comprises a mutation in BRAF or PI3CA. In some embodiments, the colon cancer is resistant to a therapy comprising an anti-EGFR antibody (e.g., cetuximab).

II. Colorectal Cancer Assigner (CRCA) System

In some embodiments, the colon cancer is classified under colorectal cancer assigner (CRCA system) as stem-like subtype. In some embodiments, the colon cancer is characterized with an over-expression of WNT signaling pathway compared to normal tissue. In some embodiments, the colon cancer is characterized with a lower expression of a differentiation marker compared to normal tissue. In some embodiments, the differentiation marker is selected from the group consisting of the expression of MUC2 and the expression of KRT20. In some embodiments, the colon cancer is characterized with a higher expression of a myoepithelial and/or mesenchymal gene compared to normal tissue.

In some embodiments, the colon cancer is classified under colorectal cancer assigner (CRCA system) as goblet-like subtype. In some embodiments, the colon cancer is characterized with a higher mRNA expression of goblet-specific MUC2 and/or TFF3.

In some embodiments, the colon cancer is classified under colorectal cancer assigner (CRCA system) as inflammatory subtype. In some embodiments, the colon cancer is characterized with a higher expression of interferon and/or cytokine compared to a normal tissue. In some embodiments, the interferon is selected from the group consisting of Type I interferon, Type II interferon, and Type III interferon. In some embodiments, the interferon is selected from the group consisting of IFN-α, IFN-β, IFN-ε, IFN-κ, IFN-ω, IFN-γ, IFN-λ1, IFN-λ2 and IFN-λ3. In some embodiments, the cytokine is selected from the group consisting of IL-2, IL-4, IL-7, IL-9, IL-15, IL-21, IL-10, IL-19, IL-20, IL-22, IL-24 (Mda-7), IL-26, erythropoietin (EPO), thrombopoietin (TPO), IL-1, IL-33, IL-18, IL-17, TGF-β1, TGF-β2 and TGF-β3.

In some embodiments, the colon cancer is classified under colorectal cancer assigner (CRCA system) as transit-amplifying subtype. In some embodiments, the colon cancer is sensitive to a therapy comprising an anti-EGFR inhibitor (e.g., cetuximab). In some embodiments, the colon cancer is not sensitive to a therapy comprising an anti-EGFR inhibitor (e.g., cetuximab). In some embodiments, the colon cancer is resistant to a therapy comprising an anti-EGFR inhibitor (e.g., cetuximab). In some embodiments, the colon cancer is not resistant to a therapy comprising an anti-EGFR inhibitor (e.g., cetuximab). In some embodiments, the colon cancer is further characterized with a higher expression of filamin A (FNLA).

In some embodiments, the colon cancer is classified under colorectal cancer assigner (CRCA system) as enterocyte subtype.

### III. Colon Cancer Molecular Subtype (CCMS) System

In some embodiments, the colon cancer is classified under the colon cancer molecular subtype (CCMS) system as C1 subtype. In some embodiments, the colon cancer is characterized with a CIN. In some embodiments, the colon cancer is characterized with a mutation in KRAS and/or TP53. In some embodiments, the colon cancer is characterized with a suppression of pathways associated with activation of the immune system and/or Epithelial-to-mesenchymal transition (EMT) compared to normal tissue.

In some embodiments, the colon cancer is classified under the colon cancer molecular subtype (CCMS) system as C2 subtype. In some embodiments, the colon cancer is characterized with a MSI and/or a CIMP. In some embodiments, the colon cancer is characterized with a mutation in BRAF. In some embodiments, the colon cancer is characterized with an alteration of a proliferative pathway. In some embodiments, the colon cancer is characterized with a suppression of the WNT pathway compared to normal tissue.

In some embodiments, the colon cancer is classified under the colon cancer molecular subtype (CCMS) system as C3 subtype. In some embodiments, the colon cancer is characterized with not having a significant level of MSI. In some embodiments, the colon cancer is characterized with a mutation in KRAS. In some embodiments, the colon cancer is characterized with an alteration of a pathway associated with the activation of immune system. In some embodiments, the colon cancer is characterized with an alteration of a pathway associated with epithelial-mesenchymal transmission.

In some embodiments, the colon cancer is classified under the colon cancer molecular subtype (CCMS) system as C4 subtype. In some embodiments, the colon cancer is characterized with both a CIN and a CIMP. In some embodiments, the colon cancer is characterized with either a CIN or a CIMP. In some embodiments, the colon cancer is characterized with at least one mutation in KRAS, BRAF and/or TP53. In some embodiments, the colon cancer is characterized with an alteration (e.g., a higher expression) of a pathway associated with Epithelial-to-mesenchymal transition (EMT) process. In some embodiments, the colon cancer is characterized with an alteration (e.g., a higher expression) of a pathway associated with serrated neoplasia pathway activation or a pathway associated with stem-cell gene expression.

In some embodiments, the colon cancer is classified under the colon cancer molecular subtype (CCMS) system as C5 subtype. In some embodiments, the colon cancer is characterized with a CIN. In some embodiments, the colon cancer is characterized with a mutation in KRAS and/or TP53. In some embodiments, the colon cancer is characterized with a higher expression of the Wnt pathway genes compared to normal tissue.

In some embodiments, the colon cancer is classified under the colon cancer molecular subtype (CCMS) system as C6 subtype. In some embodiments, the colon cancer is characterized with a CIN. In some embodiments, the colon cancer is characterized with a CIN. In some embodiments, the colon cancer is characterized with a mutation in KRAS and/or TP53. In some embodiments, the colon cancer is characterized with an alteration (e.g., a higher expression) of a pathway associated with Epithelial-to-mesenchymal transition (EMT) process. In some embodiments, the colon cancer is characterized with an alteration (e.g., a higher expression) of a pathway associated with serrated neoplasia pathway activation.

In some embodiments, the colon cancer is classified under the colon cancer molecular subtype (CCMS) system as both C1 and C5 subtype.

### IV. CRC Intrinsic Subtype (CRCIS) System

In some embodiments, the colon cancer is classified under the CRC intrinsic subtype (CRCIS) system as Type A subtype (i.e., MMR-deficient epithelial subtype). In some embodiments, the colon cancer is characterized with a MSI. In some embodiments, the colon cancer comprises at least 1, 2, 3, 4, 5, 6, 7, 8, 9, or 10 mutations. In some embodiments, the colon cancer comprises at least 1, 2, 3, 4, 5, 6, 7, 8, 9, or 10 mutations in BRAF.

In some embodiments, the colon cancer is classified under the CRC intrinsic subtype (CRCIS) system as Type B subtype (i.e., epithelial proliferative subtype). In some embodiments, the colon cancer is characterized with an epithelial phenotype. In some embodiments, the colon cancer is characterized with a higher proliferation of cancer cells compared to cancer cells of Type A or Type C subtype. In some embodiments, the colon cancer is characterized with an absence of BRAF mutation. In some embodiments, the colon cancer is characterized with microsatellite instability-high (MSI-H) or microsatellite instability-low (MSI-L).

In some embodiments, the colon cancer is classified under the CRC intrinsic subtype (CRCIS) system as Type C subtype. In some embodiments, the colon cancer is characterized with a higher EMT expression of a mesenchymal phenotype compared to Type A or Type B subtype.

In some embodiments, the colon cancer is classified under the colorectal cancer subtyping consortium (CRCSC) classification system as CMS1. In some embodiments, the colon cancer is characterized with a lesion in the right colon and/or rectum.

In some embodiments, the colon cancer is classified under the colorectal cancer subtyping consortium (CRCSC) classification system as CMS2. In some embodiments, the colon cancer is characterized with a lesion in the left colon and/or rectum. In some embodiments, the colon cancer is characterized with not having a significant level of MSI. In some embodiments, the colon cancer is characterized with a significant level of CIN. In some embodiments, the colon cancer is characterized with an alteration of a pathway. In some embodiments, the alteration of a pathway comprises WNT-signal activation and/or MYC pathway activation. In some embodiments, the alteration of a pathway comprises EGFR amplification. In some embodiments, the alteration of a pathway comprises an overexpression or mutant TP53.

In some embodiments, the colon cancer is classified under the colorectal cancer subtyping consortium (CRCSC) classification system as CMS3. In some embodiments, the colon cancer is characterized with not having a significant level of CIN. In some embodiments, the colon cancer is characterized with a significant level of CIMP. In some embodiments, the colon cancer is characterized with an alteration of a pathway. In some embodiments, the alteration of a pathway comprises WNT-signal activation and/or MYC pathway activation. In some embodiments, the alteration of a pathway comprises a mutant KRAS and/or PI3K. In some embodiments, the alteration of a pathway comprises an overexpression of IGBP2. In some embodiments, the alteration of a pathway comprises an enriched metabolism signature (e.g., mitochondrial oxidative metabolism).

In some embodiments, the colon cancer is classified under the colorectal cancer subtyping consortium (CRCSC) classification system as CMS4. In some embodiments, the colon cancer is characterized with not having a significant level of CIN. In some embodiments, the colon cancer is characterized with an alteration of a pathway. In some embodiments, the alteration of a pathway comprises TGF-β activation. In some embodiments, the alteration of a pathway comprises activation of angiogenesis, matrix remodeling and/or complement-mediated inflammation. In some embodiments, the colon cancer is in stage III. In some embodiments, the colon cancer is in stage IV.

### Methods of Treatment Based on Presence of a Biomarker

The present invention in one aspect provides methods of treating a colon cancer in an individual based on the status of one or more mTOR-activating aberrations in one or more mTOR-associated genes. In some embodiments, the one or more biomarkers are selected from the group consisting of biomarkers indicative of favorable response to treatment with an mTOR inhibitor, and biomarkers indicative of favorable response to treatment with an anti-VEGF antibody, biomarkers indicative of favorable response to treatment with a FOLFOX regimen.

A. Based on the Presence of mTOR-Activation Aberration

In some embodiments, there is provided a method of treating a colon cancer in an individual comprising administering to the individual a) an effective amount of a composition comprising nanoparticles comprising an mTOR inhibitor (such as a limus drug, e.g., sirolimus or a derivative thereof) and an albumin; b) an effective amount of anti-VEGF antibody, and c) a therapeutically effective FOLFOX regimen, wherein the individual is selected for treatment based on the individual having an mTOR-activating aberration. In some embodiments, the mTOR-activating aberration comprises a mutation of an mTOR-associated gene. In some embodiments, the mTOR-activating aberration comprises a copy number variation of an mTOR-associated gene. In some embodiments, the mTOR-activating aberration comprises an aberrant expression level of an mTOR-associated gene. In some embodiments, the mTOR-activating aberration comprises an aberrant activity level of an mTOR-associated gene. In some embodiments, the at least one mTOR-associated biomarker comprises an aberrant phosphorylation level of the protein encoded by the mTOR-associated gene. In some embodiments, the mTOR-activating aberration leads to activation of mTORC1 (including for example activation of mTORC1 but not mTORC2). In some embodiments, the mTOR-activating aberration leads to activation of mTORC2 (including for example activation of mTORC2 but not mTORC1). In some embodiments, the mTOR-activating aberration leads to activation of both mTORC1 and mTORC2. In some embodiments, the mTOR-activating aberration is in at least one mTOR-associated gene selected from the group consisting of AKT1, FLT-3, MTOR, PIK3CA, PIK3CG, TSC1, TSC2, RHEB, STK11, NF1, NF2, TP53, FGFR4, BAP1, KRAS, NRAS and PTEN. In some embodiments, the mTOR-activating aberration is assessed by gene sequencing or by immunochemistry. In some embodiments, the gene sequencing is based on sequencing of DNA in a tumor sample. In some embodiments, the gene sequencing is based on sequencing of a circulating or a cell-free DNA in a blood sample. In some embodiments, the mutational status of TFE3 is further used as a basis for selecting the individual. In some embodiments, the mutational status of TFE3 comprises translocation of TFE3. In some embodiments, the mTOR-activating aberration comprises an aberrant phosphorylation level of the protein encoded by the mTOR-associated gene. In some embodiments, the mTOR-associated gene is selected from the group consisting of AKT, S6K, S6, and 4EBP1. In some embodiments, the aberrant phosphorylation level is determined by immunohistochemistry. In some embodiments, the anti-VEGF antibody and/or at least a portion of FOLFOX regiment and the nanoparticle composition are administered sequentially. In some embodiments, the anti-VEGF antibody and/or at least a portion of FOLFOX regiment and the nanoparticle composition are administered simultaneously. In some embodiments, the anti-VEGF antibody and/or at least a portion of FOLFOX regiment and the nanoparticle composition are administered concurrently. In some embodiments, the anti-VEGF and at least a portion of FOLFOX regiment are administered sequentially. In some embodiments, the anti-VEGF antibody and at least a portion of FOLFOX regiment are administered simultaneously. In some embodiments, the anti-VEGF antibody and at least a portion of FOLFOX regiment are administered concurrently.

In some embodiments, there is provided a method of treating a colon cancer in an individual comprising: (a) assessing an mTOR-activating aberration in the individual; and (b) administering to the individual i) an effective amount of a composition comprising nanoparticles comprising an mTOR inhibitor (such as a limus drug, e.g., sirolimus or a derivative thereof) and an albumin; ii) an effective amount of anti-VEGF antibody; and iii) a therapeutically effective FOLFOX regimen, wherein the individual is selected for treatment based on having the mTOR-activating aberration. In some embodiments, the mTOR-activating aberration comprises a mutation of an mTOR-associated gene. In some embodiments, the mTOR-activating aberration comprises a copy number variation of an mTOR-associated gene. In some embodiments, the mTOR-activating aberration comprises an aberrant expression level of an mTOR-associated gene. In some embodiments, the mTOR-activating aberration comprises an aberrant activity level of an mTOR-associated gene. In some embodiments, the at least one mTOR-associated biomarker comprises an aberrant phosphorylation level of the protein encoded by the mTOR-associated gene. In some embodiments, the mTOR-activating aberration leads to activation of mTORC1 (including for example activation of mTORC1 but not mTORC2). In some embodiments, the mTOR-activating aberration leads to activation of mTORC2 (including for example activation of mTORC2 but not mTORC1). In some embodiments, the mTOR-activating aberration leads to activation of both mTORC1 and mTORC2. In some embodiments, the mTOR-activating aberration is in at least one mTOR-associated gene selected from the group consisting of AKT1, FLT-3, MTOR, PIK3CA, PIK3CG, TSC1, TSC2, RHEB, STK11, NF1, NF2, TP53, FGFR4, BAP1, KRAS, NRAS and PTEN. In some embodiments, the mTOR-activating aberration is assessed by gene sequencing or by immunohistochemistry. In some embodiments, the gene sequencing is based on sequencing of DNA in a tumor sample. In some embodiments, the gene sequencing is based on sequencing of a circulating or a cell-free DNA in a blood sample. In some embodiments, the mutational status of TFE3 is further used as a basis for selecting the individual. In some embodiments, the mutational status of TFE3 comprises translocation of TFE3. In some embodiments, the mTOR-activating aberration comprises an aberrant phosphorylation level of the protein encoded by the mTOR-associated gene. In some embodiments, the mTOR-associated gene is selected from the group consisting of AKT, S6K, S6, and 4EBP1. In some embodiments, the aberrant phosphorylation level is determined by immunohistochemistry.

In some embodiments, there is provided a method of treating a colon cancer in an individual comprising: (a) assessing an mTOR-activating aberration in the individual; (b) selecting (e.g., identifying or recommending) the individual for treatment based on the individual having the mTOR-activating aberration; and (c) administering to the individual i) an effective amount of a composition comprising nanoparticles comprising an mTOR inhibitor (such as a limus drug, e.g., sirolimus or a derivative thereof) and an albumin; ii) an effective amount of anti-VEGF antibody; and iii) a therapeutically effective FOLFOX regimen. In some embodiments, the mTOR-activating aberration comprises a mutation of an mTOR-associated gene. In some embodiments, the mTOR-activating aberration comprises a copy number variation of an mTOR-associated gene. In some embodiments, the mTOR-activating aberration comprises an aberrant expression level of an mTOR-associated gene. In some embodiments, the mTOR-activating aberration comprises an aberrant activity level of an mTOR-associated gene. In some embodiments, the at least one mTOR-associated biomarker comprises an aberrant phosphorylation level of the protein encoded by the mTOR-associated gene. In some embodiments, the mTOR-activating aberration leads to activation of mTORC1 (including for example activation of mTORC1 but not mTORC2). In some embodiments, the mTOR-activating aberration leads to activation of mTORC2 (including for example activation of mTORC2 but not mTORC1). In some embodiments, the mTOR-activating aberration leads to activation of both mTORC1 and mTORC2. In some embodiments, the mTOR-activating aberration is in at least one mTOR-associated gene selected from the group consisting of AKT1, FLT-3, MTOR, PIK3CA, PIK3CG, TSC1, TSC2, RHEB, STK11, NF1, NF2, TP53, FGFR4, BAP1, KRAS, NRAS and PTEN. In some embodiments, the mTOR-activating aberration is assessed by gene sequencing or by immunochemistry. In some embodiments, the gene sequencing is based on sequencing of DNA in a tumor sample. In some embodiments, the gene sequencing is based on sequencing of a circulating or a cell-free DNA in a blood sample. In some embodiments, the mutational status of TFE3 is further used as a basis for selecting the individual. In some embodiments, the mutational status of TFE3 comprises translocation of TFE3. In some embodiments, the mTOR-activating aberration comprises an aberrant phosphorylation level of the protein encoded by the mTOR-associated gene. In some embodiments, the mTOR-associated gene is selected from the group consisting of AKT, S6K, S6, and 4EBP1. In some embodiments, the aberrant phosphorylation level is determined by immunohistochemistry.

In some embodiments, there is provided a method of selecting (including identifying or recommending) an individual having a colon cancer for treatment with i) an effective amount of a composition comprising nanoparticles comprising an mTOR inhibitor (such as a limus drug, e.g., sirolimus or a derivative thereof) and an albumin; ii) an effective amount of anti-VEGF antibody; and iii) a therapeutically effective FOLFOX regimen, wherein the method comprises (a) assessing an mTOR-activating aberration in the individual; and (b) selecting or recommending the individual for treatment based on the individual having the mTOR-activating aberration. In some embodiments, the mTOR-activating aberration comprises a mutation of an mTOR-associated gene. In some embodiments, the mTOR-activating aberration comprises a copy number variation of an mTOR-associated gene. In some embodiments, the mTOR-activating aberration comprises an aberrant expression level of an mTOR-associated gene. In some embodiments, the mTOR-activating aberration comprises an aberrant activity level of an mTOR-associated gene. In some embodiments, the at least one mTOR-associated biomarker comprises an aberrant phosphorylation level of the protein encoded by the mTOR-associated gene. In some embodiments, the mTOR-activating aberration leads to activation of mTORC1 (including for example activation of mTORC1 but not mTORC2). In some embodiments, the mTOR-activating aberration leads to activation of mTORC2 (including for example activation of mTORC2 but not mTORC1). In some embodiments, the mTOR-activating aberration leads to activation of both mTORC1 and mTORC2. In some embodiments, the mTOR-activating aberration is in at least one mTOR-associated gene selected from the group consisting of AKT1, FLT-3, MTOR, PIK3CA, PIK3CG, TSC1, TSC2, RHEB, STK11, NF1, NF2, TP53, FGFR4, BAP1, KRAS, NRAS and PTEN. In some embodiments, the mTOR-activating aberration is assessed by gene sequencing or by immunochemistry. In some embodiments, the gene sequencing is based on sequencing of DNA in a tumor sample. In some embodiments, the gene sequencing is based on sequencing of a circulating or a cell-free DNA in a blood sample. In some embodiments, the mutational status of TFE3 is further used as a basis for selecting the individual. In some embodiments, the mutational status of TFE3 comprises translocation of TFE3. In some embodiments, the mTOR-activating aberration comprises an aberrant phosphorylation level of the protein encoded by the mTOR-associated gene. In some embodiments, the mTOR-associated gene is selected from the group consisting of AKT, S6K, S6, and 4EBP1. In some embodiments, the aberrant phosphorylation level is determined by immunohistochemistry.

In some embodiments, there is provided a method of selecting (including identifying or recommending) and treating an individual having a colon cancer, wherein the method comprises (a) assessing an mTOR-activating aberration in the individual; (b) selecting or recommending the individual for treatment based on the individual having the mTOR-activating aberration; and (c) administering to the individual i) an effective amount of a composition comprising an mTOR inhibitor (such as a limus drug, e.g., sirolimus or a derivative thereof) and an albumin; ii) an effective amount of anti-VEGF antibody; and iii) a therapeutically effective FOLFOX regimen. In some embodiments, the mTOR-activating aberration comprises a mutation of an mTOR-associated gene. In some embodiments, the mTOR-activating aberration comprises a copy number variation of an mTOR-associated gene. In some embodiments, the mTOR-activating aberration comprises an aberrant expression level of an mTOR-associated gene. In some embodiments, the mTOR-activating aberration comprises an aberrant activity level of an mTOR-associated gene. In some embodiments, the at least one mTOR-associated biomarker comprises an aberrant phosphorylation level of the protein encoded by the mTOR-associated gene. In some embodiments, the mTOR-activating aberration leads to activation of mTORC1 (including for example activation of mTORC1 but not mTORC2). In some embodiments, the mTOR-activating aberration leads to activation of mTORC2 (including for example activation of mTORC2 but not mTORC1). In some embodiments, the mTOR-activating aberration leads to activation of both mTORC1 and mTORC2. In some embodiments, the mTOR-activating aberration is in at least one mTOR-associated gene selected from the group consisting of AKT1, FLT-3, MTOR, PIK3CA, PIK3CG, TSC1, TSC2, RHEB, STK11, NF1, NF2, TP53, FGFR4, BAP1, KRAS, NRAS and PTEN. In some embodiments, the mTOR-activating aberration is assessed by gene sequencing or by immunochemistry. In some embodiments, the gene sequencing is based on sequencing of DNA in a tumor sample. In some embodiments, the gene sequencing is based on sequencing of a circulating or a cell-free DNA in a blood sample. In some embodiments, the mutational status of TFE3 is further used as a basis for selecting the individual. In some embodiments, the mutational status of TFE3 comprises translocation of TFE3. In some embodiments, the mTOR-activating aberration comprises an aberrant phosphorylation level of the protein encoded by the mTOR-associated gene. In some embodiments, the mTOR-associated gene is selected from the group consisting of AKT, S6K, S6, and 4EBP1. In some embodiments, the aberrant phosphorylation level is determined by immunohistochemistry.

Also provided herein are methods of assessing whether an individual with a colon cancer is more likely to respond or less likely to respond to treatment based on the individual having an mTOR-activating aberration, wherein the treatment comprises i) an effective amount of a composition comprising an mTOR inhibitor (such as a limus drug, e.g., sirolimus or a derivative thereof) and an albumin; ii) an effective amount of anti-VEGF antibody; and iii) a therapeutically effective FOLFOX regimen; the method comprising assessing the mTOR-activating aberration in the individual. In some embodiments, the method further comprises administering to the individual who is determined to be likely to respond to the treatment i) an effective amount of a composition comprising an mTOR inhibitor (such as a limus drug, e.g., sirolimus or a derivative thereof) and an albumin; ii) an effective amount of anti-VEGF antibody; and iii) a therapeutically effective FOLFOX regimen. In some embodiments, the presence of the mTOR-activating aberration indicates that the individual is more likely to respond to the treatment, and the absence of the mTOR-activating aberration indicates that the individual is less likely to respond to the treatment. In some embodiments, the amount of the mTOR inhibitor (such as a limus drug, e.g., sirolimus or a derivative thereof) is determined based on the status of the mTOR-activating aberration.

In some embodiments, there are also provided methods of aiding assessment of whether an individual with a colon cancer will likely respond to or is suitable for treatment based on the individual having an mTOR-activating aberration, wherein the treatment comprises i) an effective amount of a composition comprising an mTOR inhibitor (such as a limus drug, e.g., sirolimus or a derivative thereof) and an albumin; ii) an effective amount of anti-VEGF antibody; and iii) a therapeutically effective FOLFOX regimen; the method comprising assessing the mTOR-activating aberration in the individual. In some embodiments, the presence of the mTOR-activating aberration indicates that the individual will likely be responsive to the treatment, and the absence of the mTOR-activating aberration indicates that the individual is less likely to respond to the treatment. In some embodiments, the method further comprises administering to the individual i) an effective amount of a composition comprising an mTOR inhibitor (such as a limus drug, e.g., sirolimus or a derivative thereof) and an albumin; ii) an effective amount of anti-VEGF antibody; and iii) a therapeutically effective FOLFOX regimen.

In some embodiments, there is provided a method of identifying an individual with a colon cancer likely to respond to treatment comprising i) an effective amount of a composition comprising an mTOR inhibitor (such as a limus drug, e.g., sirolimus or a derivative thereof) and an albumin; ii) an effective amount of anti-VEGF antibody; and iii) a therapeutically effective FOLFOX regimen; the method comprising: a) assessing an mTOR-activating aberration in the individual; and b) identifying the individual based on the individual having the mTOR-activating aberration. In some embodiments, the method further comprises administering i) an effective amount of a composition comprising an mTOR inhibitor (such as a limus drug, e.g., sirolimus or a derivative thereof) and an albumin; ii) an effective amount of anti-VEGF antibody; and iii) a therapeutically effective FOLFOX regimen. In some embodiments, the amount of the mTOR inhibitor (such as a limus drug, e.g., sirolimus or a derivative thereof) is determined based on the status of the mTOR-activating aberration.

Also provided herein are methods of adjusting therapy treatment of an individual with a colon cancer receiving i) an effective amount of a composition comprising an mTOR inhibitor (such as a limus drug, e.g., sirolimus or a derivative thereof) and an albumin; ii) an effective amount of anti-VEGF antibody; and iii) a therapeutically effective FOLFOX regimen; the method comprising assessing an mTOR-activating aberration in a sample isolated from the individual, and adjusting the therapy treatment based on the status of the mTOR-activating aberration. In some embodiments, the amount of the mTOR inhibitor (such as a limus drug, e.g., sirolimus or a derivative thereof) is adjusted.

Also provided herein are methods of marketing a therapy comprising i) an effective amount of a composition comprising an mTOR inhibitor (such as a limus drug, e.g., sirolimus or a derivative thereof) and an albumin; ii) an effective amount of anti-VEGF antibody; and iii) a therapeutically effective FOLFOX regimen for use in a colon cancer in an individual subpopulation, the methods comprising informing a target audience about the use of the therapy for treating the individual subpopulation characterized by the individuals of such subpopulation having a sample which has an mTOR-activating aberration.

“MTOR-activating aberration” refers to a genetic aberration, an aberrant expression level and/or an aberrant activity level of one or more mTOR-associated gene that may lead to hyperactivation of the mTOR signaling pathway. “Hyperactivate” refers to increase of an activity level of a molecule (such as a protein or protein complex) or a signaling pathway (such as the mTOR a signaling pathway) to a level that is above a reference activity level or range, such as at least about any of 10%, 20%, 30%, 40%, 60%, 70%, 80%, 90%, 100%, 200%, 500% or more above the reference activity level or the median of the reference activity range. In some embodiments, the reference activity level is a clinically accepted normal activity level in a standardized test, or an activity level in a healthy individual (or tissue or cell isolated from the individual) free of the mTOR-activating aberration.

The mTOR-activating aberration contemplated herein may include one type of aberration in one mTOR-associated gene, more than one type (such as at least about any of 2, 3, 4, 5, 6, or more) of aberrations in one mTOR-associated gene, one type of aberration in more than one (such as at least about any of 2, 3, 4, 5, 6, or more) mTOR-associated genes, or more than one type (such as at least about any of 2, 3, 4, 5, 6, or more) of aberration in more than one (such as at least about any of 2, 3, 4, 5, 6, or more) mTOR-associated genes. Different types of mTOR-activating aberration may include, but are not limited to, genetic aberrations, aberrant expression levels (e.g. overexpression or under-expression), aberrant activity levels (e.g. high or low activity levels), and aberrant phosphorylation levels. In some embodiments, a genetic aberration comprises a change to the nucleic acid (such as DNA or RNA) or protein sequence (i.e. mutation) or an aberrant epigenetic feature associated with an mTOR-associated gene, including, but not limited to, coding, non-coding, regulatory, enhancer, silencer, promoter, intron, exon, and untranslated regions of the mTOR-associated gene. In some embodiments, the at least one molecule (such as a protein or protein complex) or a signaling pathway (such as the mTOR a signaling pathway) to a level that is above a reference activity level or range, such as at least about any of 10%, 20%, 30%, 40%, 60%, 70%, 80%, 90%, 100%, 200%, 500% or more above the reference activity level or the median of the reference activity range. In some embodiments, the reference activity level is a clinically accepted normal activity level in a standardized test, or an activity level in a healthy individual (or tissue or cell isolated from the individual) free of the mTOR-activating aberration.

The mTOR-activating aberration contemplated herein may include one type of aberration in one mTOR-associated gene, more than one type (such as at least about any of 2, 3, 4, 5, 6, or more) of aberrations in one mTOR-associated gene, one type of aberration in more than one (such as at least about any of 2, 3, 4, 5, 6, or more) mTOR-associated genes, or more than one type (such as at least about any of 2, 3, 4, 5, 6, or more) of aberration in more than one (such as at least about any of 2, 3, 4, 5, 6, or more) mTOR-associated genes. Different types of mTOR-activating aberration may include, but are not limited to, genetic aberrations, aberrant expression levels (e.g. overexpression or under-expression), aberrant activity levels (e.g. high or low activity levels), and aberrant phosphorylation levels. In some embodiments, a genetic aberration comprises a change to the nucleic acid (such as DNA or RNA) or protein sequence (i.e. mutation) or an aberrant epigenetic feature associated with an mTOR-associated gene, including, but not limited to, coding, non-coding, regulatory, enhancer, silencer, promoter, intron, exon, and untranslated regions of the mTOR-associated biomarker comprises an aberrant phosphorylation level of the protein encoded by the molecule (such as a protein or protein complex) or a signaling pathway (such as the mTOR a signaling pathway) to a level that is above a reference activity level or range, such as at least about any of 10%, 20%, 30%, 40%, 60%, 70%, 80%, 90%, 100%, 200%, 500% or more above the reference activity level or the median of the reference activity range. In some embodiments, the reference activity level is a clinically accepted normal activity level in a standardized test, or an activity level in a healthy individual (or tissue or cell isolated from the individual) free of the mTOR-activating aberration.

The mTOR-activating aberration contemplated herein may include one type of aberration in one mTOR-associated gene, more than one type (such as at least about any of 2, 3, 4, 5, 6, or more) of aberrations in one mTOR-associated gene, one type of aberration in more than one (such as at least about any of 2, 3, 4, 5, 6, or more) mTOR-associated genes, or more than one type (such as at least about any of 2, 3, 4, 5, 6, or more) of aberration in more than one (such as at least about any of 2, 3, 4, 5, 6, or more) mTOR-associated genes. Different types of mTOR-activating aberration may include, but are not limited to, genetic aberrations, aberrant expression levels (e.g. overexpression or under-expression), aberrant activity levels (e.g. high or low activity levels), and aberrant phosphorylation levels. In some embodiments, a genetic aberration comprises a change to the nucleic acid (such as DNA or RNA) or protein sequence (i.e. mutation) or an aberrant epigenetic feature associated with an mTOR-associated gene, including, but not limited to, coding, non-coding, regulatory, enhancer, silencer, promoter, intron, exon, and untranslated regions of the mTOR-associated gene. In some embodiments, the mTOR-activating aberration comprises a mutation of an mTOR-associated gene, including, but not limited to, deletion, frameshift, insertion, missense mutation, nonsense mutation, point mutation, silent mutation, splice site mutation, and translocation. In some embodiments, the mutation may be a loss of function mutation for a negative regulator of the mTOR signaling pathway or a gain of function mutation of a positive regulator of the mTOR signaling pathway. In some embodiments, the genetic aberration comprises a copy number variation of an mTOR-associated gene. In some embodiments, the copy number variation of the mTOR-associated gene is caused by structural rearrangement of the genome, including deletions, duplications, inversion, and translocations. In some embodiments, the genetic aberration comprises an aberrant epigenetic feature of an mTOR-associated gene, including, but not limited to, DNA methylation, hydroxymethylation, increased or decreased histone binding, chromatin remodeling, and the like.

The mTOR-activating aberration is determined in comparison to a control or reference, such as a reference sequence (such as a nucleic acid sequence or a protein sequence), a control expression (such as RNA or protein expression) level, a control activity (such as activation or inhibition of downstream targets) level, or a control protein phosphorylation level. The aberrant expression level or the aberrant activity level in an mTOR-associated gene may be above the control level (such as about any of 10%, 20%, 30%, 40%, 60%, 70%, 80%, 90%, 100%, 200%, 500% or more above the control level) if the mTOR-associated gene is a positive regulator (i.e. activator) of the mTOR signaling pathway, or below the control level (such as about any of 10%, 20%, 30%, 40%, 60%, 70%, 80%, 90% or more below the control level) if the mTOR-associated gene is a negative regulator (i.e. inhibitor) of the mTOR signaling pathway. In some embodiments, the control level (e.g., expression level or activity level) is the median level (e.g., expression level or activity level) of a control population. In some embodiments, the control population is a population having the same colon cancer (such as bladder cancer, renal cell carcinoma, or melanoma) as the individual being treated. In some embodiments, the control population is a healthy population that does not have the colon cancer (such as bladder cancer, renal cell carcinoma, or melanoma), and optionally with comparable demographic characteristics (e.g., gender, age, ethnicity, etc.) as the individual being treated. In some embodiments, the control level (e.g., expression level or activity level) is a level (e.g., expression level or activity level) of a healthy tissue from the same individual. A genetic aberration may be determined by comparing to a reference sequence, including epigenetic patterns of the reference sequence in a control sample. In some embodiments, the reference sequence is the sequence (DNA, RNA or protein sequence) corresponding to a fully functional allele of an mTOR-associated gene, such as an allele (e.g., the prevalent allele) of the mTOR-associated gene present in a healthy population of individuals that do not have the colon cancer (such as bladder cancer, renal cell carcinoma, or melanoma), but may optionally have similar demographic characteristics (such as gender, age, ethnicity etc.) as the individual being treated.

The “status” of an mTOR-activating aberration may refer to the presence or absence of the mTOR-activating aberration in one or more mTOR-associated genes, or the aberrant level (expression or activity level, including phosphorylation level of a protein). In some embodiments, the presence of a genetic aberration (such as a mutation or a copy number variation) in one or more mTOR-associated genes as compared to a control indicates that (a) the individual is more likely to respond to treatment or (b) the individual is selected for treatment. In some embodiments, the absence of a genetic aberration in an mTOR-associated gene, or a wildtype mTOR-associated gene compared to a control, indicates that (a) the individual is less likely to respond to treatment or (b) the individual is not selected for treatment. In some embodiments, an aberrant level (such as expression level or activity level, including phosphorylation level of a protein) of one or more mTOR-associated genes is correlated with the likelihood of the individual to respond to treatment. For example, a larger deviation of the level (such as expression level or activity level, including phosphorylation level of a protein) of one or more mTOR-associated genes in the direction of hyperactivating the mTOR signaling pathway indicates that the individual is more likely to respond to treatment. In some embodiments, a prediction model based on the level(s) (such as expression level or activity level, including phosphorylation level of a protein) of one or more mTOR-associated genes is used to predict (a) the likelihood of the individual to respond to treatment and (b) whether to select the individual for treatment. The prediction model, including, for example, coefficient for each level, may be obtained by statistical analysis, such as regression analysis, using clinical trial data.

The expression level, and/or activity level of the one or more mTOR-associated genes, and/or phosphorylation level of one or more proteins encoded by the one or more mTOR-associated genes, and/or the presence or absence of one or more genetic aberrations of the one or more mTOR-associated genes can be useful for determining any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); I probable or likely unsuitability of an individual to continue to receive treatment(s); (f) adjusting dosage; (g) predicting likelihood of clinical benefits.

As used herein, “based upon” includes assessing, determining, or measuring the individual's characteristics as described herein (and preferably selecting an individual suitable for receiving treatment). When the status of an mTOR-activating aberration is “used as a basis” for selection, assessing, measuring, or determining method of treatment as described herein, the mTOR-activating aberration in one or more mTOR-associated genes is determined before and/or during treatment, and the status (including presence, absence, expression level, and/or activity level of the mTOR-activating aberration) obtained is used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); I probable or likely unsuitability of an individual to continue to receive treatment(s); (f) adjusting dosage; or (g) predicting likelihood of clinical benefits.

The mTOR-activating aberration in an individual can be assessed or determined by analyzing a sample from the individual. The assessment may be based on fresh tissue samples or archived tissue samples. Suitable samples include, but are not limited to, colon cancer tissue, normal tissue adjacent to the colon cancer tissue, normal tissue distal to the colon cancer tissue, or peripheral blood lymphocytes. In some embodiments, the sample is a colon cancer tissue. In some embodiments, the sample is a biopsy containing colon cancer cells, such as fine needle aspiration of colon cancer cells or laparoscopy obtained colon cancer cells. In some embodiments, the biopsied cells are centrifuged into a pellet, fixed, and embedded in paraffin prior to the analysis. In some embodiments, the biopsied cells are flash frozen prior to the analysis. In some embodiments, the sample is a plasma sample.

In some embodiments, the sample comprises a circulating metastatic cancer cell. In some embodiments, the sample is obtained by sorting circulating tumor cells (CTCs) from blood. In some further embodiments, the CTCs have detached from a primary tumor and circulate in a bodily fluid. In some further embodiments, the CTCs have detached from a primary tumor and circulate in the bloodstream. In some embodiments, the CTCs are an indication of metastasis.

In some embodiments, the sample is mixed with an antibody that recognizes a molecule encoded by an mTOR-associated gene (such as a protein) or fragment thereof. In some embodiments, the sample is mixed with a nucleic acid that recognizes nucleic acids associated with the mTOR-associated gene (such as DNA or RNA) or fragment thereof. In some embodiments, the sample is used for sequencing analysis, such as next-generation DNA, RNA and/or exome sequencing analysis.

The mTOR-activating aberration may be assessed before the start of the treatment, at any time during the treatment, and/or at the end of the treatment. In some embodiments, the mTOR-activating aberration is assessed from about 3 days prior to the administration of an mTOR inhibitor nanoparticle composition (such as sirolimus/albumin nanoparticle composition) to about 3 days after the administration of the mTOR inhibitor nanoparticle composition in each cycle of the administration. In some embodiments, the mTOR-activating aberration is assessed on day 1 of each cycle of administration. In some embodiments, the mTOR-activating aberration is assessed in cycle 1, cycle 2 and cycle 3. In some embodiments, the mTOR-activating aberration is further assessed every 2 cycles after cycle 3.

I. mTOR-Activating Aberrations

The present application contemplates mTOR-activating aberrations in any one or more mTOR-associated genes described above, including deviations from the reference sequences (i.e. genetic aberrations), abnormal expression levels and/or abnormal activity levels of the one or more mTOR-associated genes. The present application encompasses treatments and methods based on the status of any one or more of the mTOR-activating aberrations disclosed herein.

The mTOR-activating aberrations described herein are associated with an increased (i.e. hyperactivated) mTOR signaling level or activity level. The mTOR signaling level or mTOR activity level described in the present application may include mTOR signaling in response to any one or any combination of the upstream signals described above, and may include mTOR signaling through mTORC1 and/or mTORC2, which may lead to measurable changes in any one or combinations of downstream molecular, cellular or physiological processes (such as protein synthesis, autophagy, metabolism, cell cycle arrest, apoptosis etc.). In some embodiments, the mTOR-activating aberration hyperactivates the mTOR activity by at least about any one of 10%, 20%, 30%, 40%, 60%, 70%, 80%, 90%, 100%, 200%, 500% or more above the level of mTOR activity without the mTOR-activating aberration. In some embodiments, the hyperactivated mTOR activity is mediated by mTORC1 only. In some embodiments, the hyperactivated mTOR activity is mediated by mTORC2 only. In some embodiments, the hyperactivated mTOR activity is mediated by both mTORC1 and mTORC2.

Methods of determining mTOR activity are known in the art. See, for example, Brian C G et al., Cancer Discovery, 2014, 4:554-563. The mTOR activity may be measured by quantifying any one of the downstream outputs (e.g. at the molecular, cellular, and/or physiological level) of the mTOR signaling pathway as described above. For example, the mTOR activity through mTORC1 may be measured by determining the level of phosphorylated 4EBP1 (e.g. P-S65-4EBP1), and/or the level of phosphorylated S6K1 (e.g. P-T389-S6K1), and/or the level of phosphorylated AKT1 (e.g. P-S473-AKT1). The mTOR activity through mTORC2 may be measured by determining the level of phosphorylated FoxO1 and/or FoxO3a. The level of a phosphorylated protein may be determined using any method known in the art, such as Western blot assays using antibodies that specifically recognize the phosphorylated protein of interest.

Candidate mTOR-activating aberrations may be identified through a variety of methods, for example, by literature search or by experimental methods known in the art, including, but not limited to, gene expression profiling experiments (e.g. RNA sequencing or microarray experiments), quantitative proteomics experiments, and gene sequencing experiments. For example, gene expression profiling experiments and quantitative proteomics experiments conducted on a sample collected from an individual having a colon cancer compared to a control sample may provide a list of genes and gene products (such as RNA, protein, and phosphorylated protein) that are present at aberrant levels. In some instances, gene sequencing (such as exome sequencing) experiments conducted on a sample collected from an individual having a colon cancer compared to a control sample may provide a list of genetic aberrations. Statistical association studies (such as genome-wide association studies) may be performed on experimental data collected from a population of individuals having a colon cancer to associate aberrations (such as aberrant levels or genetic aberrations) identified in the experiments with colon cancer. In some embodiments, targeted sequencing experiments (such as the ONCOPANEL™ test) are conducted to provide a list of genetic aberrations in an individual having a colon cancer.

The ONCOPANEL™ test can be used to survey exonic DNA sequences of cancer related genes and intronic regions for detection of genetic aberrations, including somatic mutations, copy number variations and structural rearrangements in DNA from various sources of samples (such as a tumor biopsy or blood sample), thereby providing a candidate list of genetic aberrations that may be mTOR-activating aberrations. In some embodiments, the mTOR-associated gene aberration is a genetic aberration or an aberrant level (such as expression level or activity level) in a gene selected from the ONCOPANEL™ test (CLIA certified). See, for example, Wagle N. et al. Cancer discovery 2.1 (2012): 82-93.

An exemplary version of ONCOPANEL™ test includes 300 cancer genes and 113 introns across 35 genes. The 300 genes included in the exemplary ONCOPANEL™ test are: ABL1, AKT1, AKT2, AKT3, ALK, ALOX12B, APC, AR, ARAF, ARID1A, ARID1B, ARID2, ASXL1, ATM, ATRX, AURKA, AURKB, AXL, B2M, BAP1, BCL2, BCL2L1, BCL2L12, BCL6, BCOR, BCORL1, BLM, BMPR1A, BRAF, BRCA1, BRCA2, BRD4, BRIP1, BUB1B, CADM2, CARD11, CBL, CBLB, CCND1, CCND2, CCND3, CCNE1, CD274, CD58, CD79B, CDC73, CDH1, CDK1, CDK2, CDK4, CDK5, CDK6, CDK9, CDKN1A, CDKN1B, CDKN1C, CDKN2A, CDKN2B, CDKN2C, CEBPA, CHEK2, CIITA, CREBBP, CRKL, CRLF2, CRTC1, CRTC2, CSF1R, CSF3R, CTNNB1, CUX1, CYLD, DDB2, DDR2, DEPDC5, DICER1, DIS3, DMD, DNMT3A, EED, EGFR, EP300, EPHA3, EPHA5, EPHA7, ERBB2, ERBB3, ERBB4, ERCC2, ERCC3, ERCC4, ERCC5, ESR1, ETV1, ETV4, ETV5, ETV6, EWSR1, EXT1, EXT2, EZH2, FAM46C, FANCA, FANCC, FANCD2, FANCE, FANCF, FANCG, FAS, FBXW7, FGFR1, FGFR2, FGFR3, FGFR4, FH, FKBP9, FLCN, FLT1, FLT3, FLT4, FUS, GATA3, GATA4, GATA6, GLI1, GLI2, GLI3, GNA11, GNAQ, GNAS, GNB2L1, GPC3, GSTM5, H3F3A, HNF1A, HRAS, ID3, IDH1, IDH2, IGF1R, IKZF1, IKZF3, INSIG1, JAK2, JAK3, KCNIP1, KDM5C, KDM6A, KDM6B, KDR, KEAP1, KIT, KRAS, LINC00894, LMO1, LMO2, LMO3, MAP2K1, MAP2K4, MAP3K1, MAPK1, MCL1, MDM2, MDM4, MECOM, MEF2B, MEN1, MET, MITF, MLH1, MLL (KMT2A), MLL2 (KTM2D), MPL, MSH2, MSH6, MTOR, MUTYH, MYB, MYBL1, MYC, MYCL1 (MYCL), MYCN, MYD88, NBN, NEGR1, NF1, NF2, NFE2L2, NFKBIA, NFKBIZ, NKX2-1, NOTCH1, NOTCH2, NPM1, NPRL2, NPRL3, NRAS, NTRK1, NTRK2, NTRK3, PALB2, PARK2, PAX5, PBRM1, PDCD1LG2, PDGFRA, PDGFRB, PHF6, PHOX2B, PIK3C2B, PIK3CA, PIK3R1, PIM1, PMS1, PMS2, PNRC1, PRAME, PRDM1, PRF1, PRKAR1A, PRKCI, PRKCZ, PRKDC, PRPF40B, PRPF8, PSMD13, PTCH1, PTEN, PTK2, PTPN11, PTPRD, QKI, RAD21, RAF1, RARA, RB1, RBL2, RECQL4, REL, RET, RFWD2, RHEB, RHPN2, ROS1, RPL26, RUNX1, SBDS, SDHA, SDHAF2, SDHB, SDHC, SDHD, SETBP1, SETD2, SF1, SF3B1, SH2B3, SLITRK6, SMAD2, SMAD4, SMARCA4, SMARCB1, SMC1A, SMC3, SMO, SOCS1, SOX2, SOX9, SQSTM1, SRC, SRSF2, STAG1, STAG2, STAT3, STAT6, STK11, SUFU, SUZ12, SYK, TCF3, TCF7L1, TCF7L2, TERC, TERT, TET2, TLR4, TNFAIP3, TP53, TSC1, TSC2, U2AF1, VHL, WRN, WT1, XPA, XPC, XPO1, ZNF217, ZNF708, ZRSR2. The intronic regions surveyed in the exemplary ONCOPANEL™ test are tiled on specific introns of ABL1, AKT3, ALK, BCL2, BCL6, BRAF, CIITA, EGFR, ERG, ETV1, EWSR1, FGFR1, FGFR2, FGFR3, FUS, IGH, IGL, JAK2, MLL, MYC, NPM1, NTRK1, PAX5, PDGFRA, PDGFRB, PPARG, RAF1, RARA, RET, ROS1, SS18, TRA, TRB, TRG, TMPRSS2. mTOR-activating aberrations (such as genetic aberration and aberrant levels) of any of the genes included in any embodiment or version of the ONCOPANEL™ test, including, but not limited to the genes and intronic regions listed above, are contemplated by the present application to serve as a basis for selecting an individual for treatment with the mTOR inhibitor nanoparticle compositions.

Whether a candidate genetic aberration or aberrant level is an mTOR-activating aberration can be determined with methods known in the art. Genetic experiments in cells (such as cell lines) or animal models may be performed to ascertain that the colon cancer-associated aberrations identified from all aberrations observed in the experiments are mTOR-activating aberrations. For example, a genetic aberration may be cloned and engineered in a cell line or animal model, and the mTOR activity of the engineered cell line or animal model may be measured and compared with corresponding cell line or animal model that do not have the genetic aberration. An increase in the mTOR activity in such experiment may indicate that the genetic aberration is a candidate mTOR-activating aberration, which may be tested in a clinical study.

### II. Genetic Aberrations

Genetic aberrations of one or more mTOR-associated genes may comprise a change to the nucleic acid (such as DNA and RNA) or protein sequence (i.e. mutation) or an epigenetic feature associated with an mTOR-associated gene, including, but not limited to, coding, non-coding, regulatory, enhancer, silencer, promoter, intron, exon, and untranslated regions of the mTOR-associated gene.

The genetic aberration may be a germline mutation (including chromosomal rearrangement), or a somatic mutation (including chromosomal rearrangement). In some embodiments, the genetic aberration is present in all tissues, including normal tissue and the colon cancer tissue, of the individual. In some embodiments, the genetic aberration is present only in the colon cancer tissue of the individual. In some embodiments, the genetic aberration is present only in a fraction of the colon cancer tissue.

In some embodiments, the mTOR-activating aberration comprises a mutation of an mTOR-associated gene, including, but not limited to, deletion, frameshift, insertion, indel, missense mutation, nonsense mutation, point mutation, single nucleotide variation (SNV), silent mutation, splice site mutation, splice variant, and translocation. In some embodiments, the mutation may be a loss of function mutation for a negative regulator of the mTOR signaling pathway or a gain of function mutation of a positive regulator of the mTOR signaling pathway.

In some embodiments, the genetic aberration comprises a copy number variation of an mTOR-associated gene. Normally, there are two copies of each mTOR-associated gene per genome. In some embodiments, the copy number of the mTOR-associated gene is amplified by the genetic aberration, resulting in at least about any of 3, 4, 5, 6, 7, 8, or more copies of the mTOR-associated gene in the genome. In some embodiments, the genetic aberration of the mTOR-associated gene results in loss of one or both copies of the mTOR-associated gene in the genome. In some embodiments, the copy number variation of the mTOR-associated gene is loss of heterozygosity of the mTOR-associated gene. In some embodiments, the copy number variation of the mTOR-associated gene is deletion of the mTOR-associated gene. In some embodiments, the copy number variation of the mTOR-associated gene is caused by structural rearrangement of the genome, including deletions, duplications, inversion, and translocation of a chromosome or a fragment thereof.

In some embodiments, the genetic aberration comprises an aberrant epigenetic feature associated with an mTOR-associated gene, including, but not limited to, DNA methylation, hydroxymethylation, aberrant histone binding, chromatin remodeling, and the like. In some embodiments, the promotor of the mTOR-associated gene is hypermethylated in the individual, for example by at least about any of 10%, 20%, 30%, 40%, 50%, 60%, 70%, 80%, 90%, or more compared to a control level (such as a clinically accepted normal level in a standardized test).

In some embodiments, the mTOR-activating aberration is a genetic aberration (such as a mutation or a copy number variation) in any one of the mTOR-associated genes described above. In some embodiments, the mTOR-activating aberration is a mutation or a copy number variation in one or more genes selected from AKT1, MTOR, PIK3CA, PIK3CG, TSC1, TSC2, RHEB, STK11, NF1, NF2, PTEN, TP53, FGFR4, and BAP1.

Genetic aberrations in mTOR-associated genes have been identified in various human cancers, including hereditary cancers and sporadic cancers. For example, germline inactivating mutations in TSC1/2 cause tuberous sclerosis, and patients with this condition are present with lesions that include skin and brain hamartomas, renal angiomyolipomas, and renal cell carcinoma (RCC) (Krymskaya V P et al. 2011 FASEB Journal 25(6): 1922-1933). PTEN hamartoma tumor syndrome (PHTS) is linked to inactivating germline PTEN mutations and is associated with a spectrum of clinical manifestations, including breast cancer, endometrial cancer, follicular thyroid cancer, hamartomas, and RCC (Legendre C. et al. 2003 Transplantation proceedings 35(3 Suppl): 151S-153S). In addition, sporadic kidney cancer has also been shown to harbor somatic mutations in several genes in the PI3K-Akt-mTOR pathway (e.g. AKT1, MTOR, PIK3CA, PTEN, RHEB, TSC1, TSC2) (Power L A, 1990 Am. J. Hosp. Pharm. 475.5: 1033-1049; Badesch D B et al. 2010 Chest 137(2): 376-3871; Kim J C & Steinberg G D, 2001, The Journal of urology, 165(3): 745-756; McKiernan J. et al. 2010, J. Urol. 183(Suppl 4)). Of the top significantly mutated genes identified by the Cancer Genome Atlas in clear cell renal cell carcinoma, the mutation rate is about 17% for gene mutations that converge on mTORC1 activation (Cancer Genome Atlas Research Network. “Comprehensive molecular characterization of clear cell renal cell carcinoma.” 2013 Nature 499: 43-49). Genetic aberrations in mTOR-associated genes have been found to confer sensitivity in individuals having cancer to treatment with a limus drug. See, for example, Wagle et al., N. Eng. J. Med. 2014, 371:1426-33; Iyer et al., Science 2012, 338: 221; Wagle et al. Cancer Discovery 2014, 4:546-553; Grabiner et al., Cancer Discovery 2014, 4:554-563; Dickson et al. Intl Cancer 2013, 132(7): 1711-1717, and Lim et al, J Clin. Oncol. 33, 2015 suppl; abstr 11010. Genetic aberrations of mTOR-associated genes described by the above references are incorporated herein. Exemplary genetic aberrations in some mTOR-associated genes are described below, and it is understood that the present application is not limited to the exemplary genetic aberrations described herein.

In some embodiments, the mTOR-activating aberration comprises a genetic aberration in MTOR. In some embodiments, the genetic aberration comprises an activating mutation of MTOR. In some embodiments, the activating mutation of MTOR is at one or more positions (such as about any one of 1, 2, 3, 4, 5, 6, or more positions) in the protein sequence of MTOR selected from the group consisting of N269, L1357, N1421, L1433, A1459, L1460, C1483, E1519, K1771, E1799, F1888, I1973, T1977, V2006, E2014, I2017, N2206, L2209, A2210, S2215, L2216, R2217, L2220, Q2223, A2226, E2419, L2431, I2500, R2505, and D2512. In some embodiments, the activating mutation of MTOR is one or more missense mutations (such as about any one of 1, 2, 3, 4, 5, 6, or more mutations) selected from the group consisting of N269S, L1357F, N1421D, L1433S, A1459P, L1460P, C1483F, C1483R, C1483W, C1483Y, E1519T, K1771R, E1799K, F1888I, F1888I L, I1973F, T1977R, T1977K, V2006I, E2014K, I2017T, N2206S, L2209V, A2210P, S2215Y, S2215F, S2215P, L2216P, R2217W, L2220F, Q2223K, A2226S, E2419K, L2431P, I2500M, R2505P, and D2512H. In some embodiments, the activating mutation of MTOR disrupts binding of MTOR with RHEB. In some embodiments, the activating mutation of MTOR disrupts binding of MTOR with DEPTOR.

In some embodiments, the mTOR-activating aberration comprises a genetic aberration in TSC1 or TSC2. In some embodiments, the genetic aberration comprises a loss of heterozygosity of TSC1 or TSC2. In some embodiments, the genetic aberration comprises a loss of function mutation in TSC1 or TSC2. In some embodiments, the loss of function mutation is a frameshift mutation or a nonsense mutation in TSC1 or TSC2. In some embodiments, the loss of function mutation is a frameshift mutation c.1907_1908del in TSC1. In some embodiments, the loss of function mutation is a splice variant of TSC1: c.1019+1G>A. In some embodiments, the loss of function mutation is the nonsense mutation c.1073G>A in TSC2, and/or p.Trp103* in TSC1. In some embodiments, the loss of function mutation comprises a missense mutation in TSC1 or in TSC2. In some embodiments, the missense mutation is in position A256 of TSC1, and/or position Y719 of TSC2. In some embodiments, the missense mutation comprises A256V in TSC1 or Y719H in TSC2.

In some embodiments, the mTOR-activating aberration comprises a genetic aberration in RHEB. In some embodiments, the genetic aberration comprises a loss of function mutation in RHEB. In some embodiments, the loss of function mutation is at one or more positions in the protein sequence of RHEB selected from Y35 and E139. In some embodiments, the loss of function mutation in RHEB is selected from Y35N, Y35C, Y35H and E139K.

In some embodiments, the mTOR-activating aberration comprises a genetic aberration in NF1. In some embodiments, the genetic aberration comprises a loss of function mutation in NF1. In some embodiments, the loss of function mutation in NF1 is a missense mutation at position D1644 in NF1. In some embodiments, the missense mutation is D1644A in NF1.

In some embodiments, the mTOR-activating aberration comprises a genetic aberration in NF2. In some embodiments, the genetic aberration comprises a loss of function mutation in NF2. In some embodiments, the loss of function mutation in NF2 is a nonsense mutation. In some embodiments, the nonsense mutation in NF2 is c.863C>G.

In some embodiments, the mTOR-activating aberration comprises a genetic aberration in PTEN. In some embodiments, the genetic aberration comprises a deletion of PTEN in the genome. In some embodiments, the genetic aberration comprises a loss of function mutation in PTEN. In some embodiments, the loss of function mutation comprises a missense mutation, a nonsense mutation or a frameshift mutation. In some embodiments, the mutation comprises at a position in PTEN selected from the group consisting of K125E, K125X, E150Q, D153Y D153N K62R, Y65C, V217A, and N323K. In some embodiments, the genetic aberration comprises a loss of heterozygosity (LOH) at the PTEN locus.

In some embodiments, the mTOR-activating aberration comprises a genetic aberration in PI3K. In some embodiments, the genetic aberration comprises a loss of function mutation in PIK3CA or PIK3CG. In some embodiments, the loss of function mutation comprises a missense mutation at a position in PIK3CA selected from the group consisting of E542, I844, and H1047. In some embodiments, the loss of function mutation comprises a missense in PIK3CA selected from the group consisting of E542K, I844V, and H1047R.

In some embodiments, the mTOR-activating aberration comprises a genetic aberration in AKT1. In some embodiments, the genetic aberration comprises an activating mutation in AKT1. In some embodiments, the activating mutation is a missense mutation in position H238 in AKT1. In some embodiments, the missense mutation is H238Y in AKT1.

In some embodiments, the mTOR-activating aberration comprises a genetic aberration in TP53. In some embodiments, the genetic aberration comprises a loss of function mutation in TP53. In some embodiments, the loss of function mutation is a frameshift mutation in TP53, such as A39fs*5.

The genetic aberrations of the mTOR-associated genes may be assessed based on a sample, such as a sample from the individual and/or reference sample. In some embodiments, the sample is a tissue sample or nucleic acids extracted from a tissue sample. In some embodiments, the sample is a cell sample (for example a CTC sample) or nucleic acids extracted from a cell sample. In some embodiments, the sample is a tumor biopsy. In some embodiments, the sample is a tumor sample or nucleic acids extracted from a tumor sample. In some embodiments, the sample is a biopsy sample or nucleic acids extracted from the biopsy sample. In some embodiments, the sample is a Formaldehyde Fixed-Paraffin Embedded (FFPE) sample or nucleic acids extracted from the FFPE sample. In some embodiments, the sample is a blood sample. In some embodiments, cell-free DNA is isolated from the blood sample. In some embodiments, the biological sample is a plasma sample or nucleic acids extracted from the plasma sample.

The genetic aberrations of the mTOR-associated gene may be determined by any method known in the art. See, for example, Dickson et al. Int. J. Cancer, 2013, 132(7): 1711-1717; Wagle N. Cancer Discovery, 2014, 4:546-553; and Cancer Genome Atlas Research Network. Nature 2013, 499: 43-49. Exemplary methods include, but are not limited to, genomic DNA sequencing, bisulfate sequencing or other DNA sequencing-based methods using Sanger sequencing or next generation sequencing platforms; polymerase chain reaction assays; in situ hybridization assays; and DNA microarrays. The epigenetic features (such as DNA methylation, histone binding, or chromatin modifications) of one or more mTOR-associated genes from a sample isolated from the individual may be compared with the epigenetic features of the one or more mTOR-associated genes from a control sample. The nucleic acid molecules extracted from the sample can be sequenced or analyzed for the presence of the mTOR-activating genetic aberrations relative to a reference sequence, such as the wildtype sequences of AKT1, FLT-3, MTOR, PIK3CA, PIK3CG, TSC1, TSC2, RHEB, STK11, NF1, NF2, TP53, FGFR4, BAP1, KRAS, NRAS and PTEN.

In some embodiments, the genetic aberration of an mTOR-associated gene is assessed using cell-free DNA sequencing methods. In some embodiments, the genetic aberration of an mTOR-associated gene is assessed using next-generation sequencing. In some embodiments, the genetic aberration of an mTOR-associated gene isolated from a blood sample is assessed using next-generation sequencing. In some embodiments, the genetic aberration of an mTOR-associated gene is assessed using exome sequencing. In some embodiments, the genetic aberration of an mTOR-associated gene is assessed using fluorescence in-situ hybridization analysis. In some embodiments, the genetic aberration of an mTOR-associated gene is assessed prior to initiation of the methods of treatment described herein. In some embodiments, the genetic aberration of an mTOR-associated gene is assessed after initiation of the methods of treatment described herein. In some embodiments, the genetic aberration of an mTOR-associated gene is assessed prior to and after initiation of the methods of treatment described herein.

### III. Aberrant Levels

An aberrant level of an mTOR-associated gene may refer to an aberrant expression level or an aberrant activity level.

Aberrant expression level of an mTOR-associated gene comprises an increase or decrease in the level of a molecule encoded by the mTOR-associated gene compared to the control level. The molecule encoded by the mTOR-associated gene may include RNA transcript(s) (such as mRNA), protein isoform(s), phosphorylated and/or dephosphorylated states of the protein isoform(s), ubiquitinated and/or de-ubiquitinated states of the protein isoform(s), membrane localized (e.g. myristoylated, palmitoylated, and the like) states of the protein isoform(s), other post-translationally modified states of the protein isoform(s), or any combination thereof.

Aberrant activity level of an mTOR-associated gene comprises enhancement or repression of a molecule encoded by any downstream target gene of the mTOR-associated gene, including epigenetic regulation, transcriptional regulation, translational regulation, post-translational regulation, or any combination thereof of the downstream target gene. Additionally, activity of an mTOR-associated gene comprises downstream cellular and/or physiological effects in response to the mTOR-activating aberration, including, but not limited to, protein synthesis, cell growth, proliferation, signal transduction, mitochondria metabolism, mitochondria biogenesis, stress response, cell cycle arrest, autophagy, microtubule organization, and lipid metabolism.

In some embodiments, the mTOR-activating aberration (e.g. aberrant expression level) comprises an aberrant protein phosphorylation level. In some embodiments, the aberrant phosphorylation level is in a protein encoded by an mTOR-associated gene selected from the group consisting of AKT, TSC2, mTOR, PRAS40, S6K, S6, and 4EBP1. Exemplary phosphorylated species of mTOR-associated genes that may serve as relevant biomarkers include, but are not limited to, AKT S473 phosphorylation, PRAS40 T246 phosphorylation, mTOR S2448 phosphorylation, 4EBP1 T36 phosphorylation, S6K T389 phosphorylation, 4EBP1 T70 phosphorylation, and S6 S235 phosphorylation. In some embodiments, the individual is selected for treatment if the protein in the individual is phosphorylated. In some embodiments, the individual is selected for treatment if the protein in the individual is not phosphorylated. In some embodiments, the phosphorylation status of the protein is determined by immunohistochemistry.

The levels (such as expression levels and/or activity levels) of an mTOR-associated gene in an individual may be determined based on a sample (e.g., sample from the individual or reference sample). In some embodiments, the sample is from a tissue, organ, cell, or tumor. In some embodiments, the sample is a biological sample. In some embodiments, the biological sample is a biological fluid sample or a biological tissue sample. In further embodiments, the biological fluid sample is a bodily fluid. In some embodiments, the sample is a colon cancer tissue, normal tissue adjacent to said colon cancer tissue, normal tissue distal to said colon cancer tissue, blood sample, or other biological sample. In some embodiments, the sample is a fixed sample. Fixed samples include, but are not limited to, a formalin fixed sample, a paraffin-embedded sample, or a frozen sample. In some embodiments, the sample is a biopsy containing colon cancer cells. In a further embodiment, the biopsy is a fine needle aspiration of colon cancer cells. In a further embodiment, the biopsy is laparoscopy obtained colon cancer cells. In some embodiments, the biopsied cells are centrifuged into a pellet, fixed, and embedded in paraffin. In some embodiments, the biopsied cells are flash frozen. In some embodiments, the biopsied cells are mixed with an antibody that recognizes a molecule encoded by the mTOR-associated gene. In some embodiments, the at least one mTOR-associated gene comprises enhancement or repression of a molecule encoded by any downstream target gene of the mTOR-associated gene, including epigenetic regulation, transcriptional regulation, translational regulation, post-translational regulation, or any combination thereof of the downstream target gene. Additionally, activity of an mTOR-associated gene comprises downstream cellular and/or physiological effects in response to the mTOR-activating aberration, including, but not limited to, protein synthesis, cell growth, proliferation, signal transduction, mitochondria metabolism, mitochondria biogenesis, stress response, cell cycle arrest, autophagy, microtubule organization, and lipid metabolism.

In some embodiments, the mTOR-activating aberration (e.g. aberrant expression level) comprises an aberrant protein phosphorylation level. In some embodiments, the aberrant phosphorylation level is in a protein encoded by an mTOR-associated gene selected from the group consisting of AKT, TSC2, mTOR, PRAS40, S6K, S6, and 4EBP1. Exemplary phosphorylated species of mTOR-associated genes that may serve as relevant biomarkers include, but are not limited to, AKT S473 phosphorylation, PRAS40 T246 phosphorylation, mTOR S2448 phosphorylation, 4EBP1 T36 phosphorylation, S6K T389 phosphorylation, 4EBP1 T70 phosphorylation, and S6 S235 phosphorylation. In some embodiments, the individual is selected for treatment if the protein in the individual is phosphorylated. In some embodiments, the individual is selected for treatment if the protein in the individual is not phosphorylated. In some embodiments, the phosphorylation status of the protein is determined by immunohistochemistry.

Aberrant levels of mTOR-associates genes have been associated with cancer. For example, high levels (74%) of phosphorylated mTOR expression were found in human bladder cancer tissue array, and phosphorylated mTOR intensity was associated with reduced survival (Hansel D E et al, (2010) Am. J. Pathol. 176: 3062-3072). mTOR expression was shown to increase as a function of the disease stage in progression from superficial disease to invasive bladder cancer, as evident by activation of pS6-kinase, which was activated in 54 of 70 cases (77%) of T2 muscle-invasive bladder tumors (Seager C M et al, (2009) Cancer Prev. Res. (Phila) 2, 1008-1014). The mTOR signaling pathway is also known to be hyperactivated in pulmonary arterial hypertension.

The levels (such as expression levels and/or activity levels) of an mTOR-associated gene in an individual may be determined based on a sample (e.g., sample from the individual or reference sample). In some embodiments, the sample is from a tissue, organ, cell, or tumor. In some embodiments, the sample is a biological sample. In some embodiments, the biological sample is a biological fluid sample or a biological tissue sample. In further embodiments, the biological fluid sample is a bodily fluid. In some embodiments, the sample is a colon cancer tissue, normal tissue adjacent to said colon cancer tissue, normal tissue distal to said colon cancer tissue, blood sample, or other biological sample. In some embodiments, the sample is a fixed sample. Fixed samples include, but are not limited to, a formalin fixed sample, a paraffin-embedded sample, or a frozen sample. In some embodiments, the sample is a biopsy containing colon cancer cells. In a further embodiment, the biopsy is a fine needle aspiration of colon cancer cells. In a further embodiment, the biopsy is laparoscopy obtained colon cancer cells. In some embodiments, the biopsied cells are centrifuged into a pellet, fixed, and embedded in paraffin. In some embodiments, the biopsied cells are flash frozen. In some embodiments, the biopsied cells are mixed with an antibody that recognizes a molecule encoded by the mTOR-associated biomarker comprises an aberrant phosphorylation level of the protein encoded by the mTOR-associated gene comprises enhancement or repression of a molecule encoded by any downstream target gene of the mTOR-associated gene, including epigenetic regulation, transcriptional regulation, translational regulation, post-translational regulation, or any combination thereof of the downstream target gene. Additionally, activity of an mTOR-associated gene comprises downstream cellular and/or physiological effects in response to the mTOR-activating aberration, including, but not limited to, protein synthesis, cell growth, proliferation, signal transduction, mitochondria metabolism, mitochondria biogenesis, stress response, cell cycle arrest, autophagy, microtubule organization, and lipid metabolism.

In some embodiments, the mTOR-activating aberration (e.g. aberrant expression level) comprises an aberrant protein phosphorylation level. In some embodiments, the aberrant phosphorylation level is in a protein encoded by an mTOR-associated gene selected from the group consisting of PTEN, AKT, TSC2, mTOR, PRAS40, S6K, S6, and 4EBP1. Exemplary phosphorylated species of mTOR-associated genes that may serve as relevant biomarkers include, but are not limited to, PTEN Thr366, Ser370, Ser380, Thr382, Thr383, and/or Ser385 phosphorylation, AKT S473 phosphorylation, PRAS40 T246 phosphorylation, mTOR S2448 phosphorylation, 4EBP1 T36 phosphorylation, S6K T389 phosphorylation, 4EBP1 T70 phosphorylation, and S6 S235 phosphorylation. In some embodiments, the individual is selected for treatment if the protein in the individual is phosphorylated. In some embodiments, the individual is selected for treatment if the protein in the individual is not phosphorylated. In some embodiments, the phosphorylation status of the protein is determined by immunohistochemistry.

Aberrant levels of mTOR-associates genes have been associated with cancer. For example, high levels (74%) of phosphorylated mTOR expression were found in human bladder cancer tissue array, and phosphorylated mTOR intensity was associated with reduced survival (Hansel D E et al, (2010) Am. J. Pathol. 176: 3062-3072). mTOR expression was shown to increase as a function of the disease stage in progression from superficial disease to invasive bladder cancer, as evident by activation of pS6-kinase, which was activated in 54 of 70 cases (77%) of T2 muscle-invasive bladder tumors (Seager C M et al, (2009) Cancer Prev. Res. (Phila) 2, 1008-1014). The mTOR signaling pathway is also known to be hyperactivated in pulmonary arterial hypertension.

The levels (such as expression levels and/or activity levels) of an mTOR-associated gene in an individual may be determined based on a sample (e.g., sample from the individual or reference sample). In some embodiments, the sample is from a tissue, organ, cell, or tumor. In some embodiments, the sample is a biological sample. In some embodiments, the biological sample is a biological fluid sample or a biological tissue sample. In further embodiments, the biological fluid sample is a bodily fluid. In some embodiments, the sample is a colon cancer tissue, normal tissue adjacent to said colon cancer tissue, normal tissue distal to said colon cancer tissue, blood sample, or other biological sample. In some embodiments, the sample is a fixed sample. Fixed samples include, but are not limited to, a formalin fixed sample, a paraffin-embedded sample, or a frozen sample. In some embodiments, the sample is a biopsy containing colon cancer cells. In a further embodiment, the biopsy is a fine needle aspiration of colon cancer cells. In a further embodiment, the biopsy is laparoscopy obtained colon cancer cells. In some embodiments, the biopsied cells are centrifuged into a pellet, fixed, and embedded in paraffin. In some embodiments, the biopsied cells are flash frozen. In some embodiments, the biopsied cells are mixed with an antibody that recognizes a molecule encoded by the mTOR-associated gene. In some embodiments, a biopsy is taken to determine whether an individual has a colon cancer and is then used as a sample. In some embodiments, the sample comprises surgically obtained colon cancer cells. In some embodiments, samples may be obtained at different times than when the determining of expression levels of mTOR-associated gene occurs.

In some embodiments, the sample comprises a circulating metastatic cancer cell. In some embodiments, the sample is obtained by sorting circulating tumor cells (CTCs) from blood. In a further embodiment, the CTCs have detached from a primary tumor and circulate in a bodily fluid. In yet a further embodiment, the CTCs have detached from a primary tumor and circulate in the bloodstream. In a further embodiment, the CTCs are an indication of metastasis.

In some embodiments, the level of a protein encoded by an mTOR-associated gene is determined to assess the aberrant expression level of the mTOR-associated gene. In some embodiments, the level of a protein encoded by a downstream target gene of an mTOR-associated gene is determined to assess the aberrant activity level of the mTOR-associated gene. In some embodiments, protein level is determined using one or more antibodies specific for one or more epitopes of the individual protein or proteolytic fragments thereof. Detection methodologies suitable for use in the practice of the invention include, but are not limited to, immunohistochemistry, enzyme linked immunosorbent assays (ELISAs), Western blotting, mass spectroscopy, and immuno-PCR. In some embodiments, levels of protein(s) encoded by the mTOR-associated gene and/or downstream target gene(s) thereof in a sample are normalized (such as divided) by the level of a housekeeping protein (such as glyceraldehyde 3-phosphate dehydrogenase, or GAPDH) in the same sample.

In some embodiments, the level of an mRNA encoded by an mTOR-associated gene is determined to assess the aberrant expression level of the mTOR-associated gene. In some embodiments, the level of an mRNA encoded by a downstream target gene of an mTOR-associated gene is determined to assess the aberrant activity level of the mTOR-associated gene. In some embodiments, a reverse-transcription (RT) polymerase chain reaction (PCR) assay (including a quantitative RT-PCR assay) is used to determine the mRNA levels. In some embodiments, a gene chip or next-generation sequencing methods (such as RNA (cDNA) sequencing or exome sequencing) are used to determine the levels of RNA (such as mRNA) encoded by the mTOR-associated gene and/or downstream target genes thereof. In some embodiments, an mRNA level of the mTOR-associated gene and/or downstream target genes thereof in a sample are normalized (such as divided) by the mRNA level of a housekeeping gene (such as GAPDH) in the same sample.

The levels of an mTOR-associated gene may be a high level or a low level as compared to a control or reference. In some embodiments, wherein the mTOR-associated gene is a positive regulator of the mTOR activity (such as mTORC1 and/or mTORC2 activity), the aberrant level of the mTOR associated gene is a high level compared to the control. In some embodiments, wherein the mTOR-associated gene is a negative regulator of the mTOR activity (such as mTORC1 and/or mTORC2 activity), the aberrant level of the mTOR associated gene is a low level compared to the control.

In some embodiments, the level of the mTOR-associated gene in an individual is compared to the level of the mTOR-associated gene in a control sample. In some embodiments, the level of the mTOR-associated gene in an individual is compared to the level of the mTOR-associated gene in multiple control samples. In some embodiments, multiple control samples are used to generate a statistic that is used to classify the level of the mTOR-associated gene in an individual with a colon cancer.

The classification or ranking of the level (i.e., high or low) of the mTOR-associated gene may be determined relative to a statistical distribution of control levels. In some embodiments, the classification or ranking is relative to a control sample, such as a normal tissue (e.g. peripheral blood mononuclear cells), or a normal epithelial cell sample (e.g. a buccal swap or a skin punch) obtained from the individual. In some embodiments, the level of the mTOR-associated gene is classified or ranked relative to a statistical distribution of control levels. In some embodiments, the level of the mTOR-associated gene is classified or ranked relative to the level from a control sample obtained from the individual.

Control samples can be obtained using the same sources and methods as non-control samples. In some embodiments, the control sample is obtained from a different individual (for example an individual not having the colon cancer; an individual having a benign or less advanced form of a disease corresponding to the colon cancer; and/or an individual sharing similar ethnic, age, and gender). In some embodiments when the sample is a tumor tissue sample, the control sample may be a non-cancerous sample from the same individual. In some embodiments, multiple control samples (for example from different individuals) are used to determine a range of levels of the mTOR-associated genes in a particular tissue, organ, or cell population.

In some embodiments, the control sample is a cultured tissue or cell that has been determined to be a proper control. In some embodiments, the control is a cell that does not have the mTOR-activating aberration. In some embodiments, a clinically accepted normal level in a standardized test is used as a control level for determining the aberrant level of the mTOR-associated gene. In some embodiments, the level of the mTOR-associated gene or downstream target genes thereof in the individual is classified as high, medium or low according to a scoring system, such as an immunohistochemistry-based scoring system.

In some embodiments, the level of the mTOR-associated gene is determined by measuring the level of the mTOR-associated gene in an individual and comparing to a control or reference (e.g., the median level for the given patient population or level of a second individual). For example, if the level of the mTOR-associated gene for the single individual is determined to be above the median level of the patient population, that individual is determined to have high expression level of the mTOR-associated gene. Alternatively, if the level of the mTOR-associated gene for the single individual is determined to be below the median level of the patient population, that individual is determined to have low expression level of the mTOR-associated gene. In some embodiments, the individual is compared to a second individual and/or a patient population which is responsive to the treatment. In some embodiments, the individual is compared to a second individual and/or a patient population which is not responsive to the treatment. In some embodiments, the levels are determined by measuring the level of a nucleic acid encoded by the mTOR-associated gene and/or a downstream target gene thereof. For example, if the level of a molecule (such as an mRNA or a protein) encoded by the mTOR-associated gene for the single individual is determined to be above the median level of the patient population, that individual is determined to have a high level of the molecule (such as mRNA or protein) encoded by the mTOR-associated gene. Alternatively, if the level of a molecule (such as an mRNA or a protein) encoded by the mTOR-associated gene for the single individual is determined to be below the median level of the patient population, that individual is determined to have a low level of the molecule (such as mRNA or protein) encoded by the mTOR-associated gene.

In some embodiments, the control level of an mTOR-associated gene is determined by obtaining a statistical distribution of the levels of mTOR-associated gene. In some embodiments, the level of the mTOR-associated gene is classified or ranked relative to control levels or a statistical distribution of control levels.

In some embodiments, bioinformatics methods are used for the determination and classification of the levels of the mTOR-associated gene, including the levels of downstream target genes of the mTOR-associated gene as a measure of the activity level of the mTOR-associated gene. Numerous bioinformatics approaches have been developed to assess gene set expression profiles using gene expression profiling data. Methods include but are not limited to those described in Segal, E. et al. Nat. Genet. 34:66-176 (2003); Segal, E. et al. Nat. Genet. 36:1090-1098 (2004); Barry, W. T. et al. Bioinformatics 21:1943-1949 (2005); Tian, L. et al. Proc Nat'l Acad Sci USA 102:13544-13549 (2005); Novak B A and Jain A N. Bioinformatics 22:233-41 (2006); Maglietta R et al. Bioinformatics 23:2063-72 (2007); Bussemaker H J, BMC Bioinformatics 8 Suppl 6:S6 (2007).

In some embodiments, the control level is a pre-determined threshold level. In some embodiments, mRNA level is determined, and a low level is an mRNA level less than about any of 1, 0.9, 0.8, 0.7, 0.6, 0.5, 0.4, 0.3, 0.2, 0.1, 0.05, 0.02, 0.01, 0.005, 0.002, 0.001 or less time that of what is considered as clinically normal or of the level obtained from a control. In some embodiments, a high level is an mRNA level more than about 1.1, 1.2, 1.3, 1.5, 1.7, 2, 2.2, 2.5, 2.7, 3, 5, 7, 10, 20, 50, 70, 100, 200, 500, 1000 times or more than 1000 times that of what is considered as clinically normal or of the level obtained from a control.

In some embodiments, protein expression level is determined, for example by Western blot or an enzyme-linked immunosorbent assay (ELISA). For example, the criteria for low or high levels can be made based on the total intensity of a band on a protein gel corresponding to the protein encoded by the mTOR-associated gene that is blotted by an antibody that specifically recognizes the protein encoded by the mTOR-associated gene, and normalized (such as divided) by a band on the same protein gel of the same sample corresponding to a housekeeping protein (such as GAPDH) that is blotted by an antibody that specifically recognizes the housekeeping protein (such as GAPDH). In some embodiments, the protein level is low if the protein level is less than about any of 1, 0.9, 0.8, 0.7, 0.6, 0.5, 0.4, 0.3, 0.2, 0.1, 0.05, 0.02, 0.01, 0.005, 0.002, 0.001 or less time of what is considered as clinically normal or of the level obtained from a control. In some embodiments, the protein level is high if the protein level is more than about any of 1.1, 1.2, 1.3, 1.5, 1.7, 2, 2.2, 2.5, 2.7, 3, 5, 7, 10, 20, 50, or 100 times or more than 100 times of what is considered as clinically normal or of the level obtained from a control.

In some embodiments, protein expression level is determined, for example by immunohistochemistry. For example, the criteria for low or high levels can be made based on the number of positive staining cells and/or the intensity of the staining, for example by using an antibody that specifically recognizes the protein encoded by the mTOR-associated gene. In some embodiments, the level is low if less than about 1%, 5%, 10%, 15%, 20%, 25%, 30%, 35%, 40%, 45%, or 50% cells have positive staining. In some embodiments, the level is low if the staining is 1%, 5%, 10%, 15%, 20%, 25%, 30%, 35%, 40%, 45%, or 50% less intense than a positive control staining. In some embodiments, the level is high if more than about 40%, 45%, 50%, 55%, 60%, 65%, 70%, 75%, 80%, 85%, or 90%, cells have positive staining. In some embodiments, the level is high if the staining is as intense as positive control staining. In some embodiments, the level is high if the staining is 80%, 85%, or 90% as intense as positive control staining.

In some embodiments, the scoring is based on an “H-score” as described in US Pat. Pub. No. 2013/0005678. An H-score is obtained by the formula: 3×percentage of strongly staining cells+2×percentage of moderately staining cells+percentage of weakly staining cells, giving a range of 0 to 300.

In some embodiments, strong staining, moderate staining, and weak staining are calibrated levels of staining, wherein a range is established and the intensity of staining is binned within the range. In some embodiments, strong staining is staining above the 75^{th} percentile of the intensity range, moderate staining is staining from the 25^{th} to the 75^{th} percentile of the intensity range, and low staining is staining is staining below the 25^{th} percentile of the intensity range. In some aspects one skilled in the art, and familiar with a particular staining technique, adjusts the bin size and defines the staining categories.

In some embodiments, the label high staining is assigned where greater than 50% of the cells stained exhibited strong reactivity, the label no staining is assigned where no staining was observed in less than 50% of the cells stained, and the label low staining is assigned for all of other cases.

In some embodiments, the assessment and/or scoring of the genetic aberration or the level of the mTOR-associated gene in a sample, patient, etc., is performed by one or more experienced clinicians, i.e., those who are experienced with the mTOR-associated gene expression and the mTOR-associated gene product staining patterns. For example, in some embodiments, the clinician(s) is blinded to clinical characteristics and outcome for the samples, patients, etc. being assessed and scored.

In some embodiments, level of protein phosphorylation is determined. The phosphorylation status of a protein may be assessed from a variety of sample sources. In some embodiments, the sample is a tumor biopsy. The phosphorylation status of a protein may be assessed via a variety of methods. In some embodiments, the phosphorylation status is assessed using immunohistochemistry. The phosphorylation status of a protein may be site specific. The phosphorylation status of a protein may be compared to a control sample. In some embodiments, the phosphorylation status is assessed prior to initiation of the methods of treatment described herein. In some embodiments, the phosphorylation status is assessed after initiation of the methods of treatment described herein. In some embodiments, the phosphorylation status is assessed prior to and after initiation of the methods of treatment described herein.

Further provided herein are methods of directing treatment of a colon cancer by delivering a sample to a diagnostic lab for determination of the level of an mTOR-associated gene; providing a control sample with a known level of the mTOR-associated gene; providing an antibody to a molecule encoded by the mTOR-associated gene or an antibody to a molecule encoded by a downstream target gene of the mTOR-associated gene; individually contacting the sample and control sample with the antibody, and/or detecting a relative amount of antibody binding, wherein the level of the sample is used to provide a conclusion that a patient should receive a treatment with any one of the methods described herein.

Also provided herein are methods of directing treatment of a colon cancer, further comprising reviewing or analyzing data relating to the status (such as presence/absence or level) of an mTOR-activating aberration in a sample; and providing a conclusion to an individual, such as a health care provider or a health care manager, about the likelihood or suitability of the individual to respond to a treatment, the conclusion being based on the review or analysis of data. In one aspect of the invention a conclusion is the transmission of the data over a network.

### IV. Resistance Biomarkers

Genetic aberrations and aberrant levels of certain genes may be associated with resistance to the treatment methods described herein. In some embodiments, the individual having an aberration (such as genetic aberration or aberrant level) in a resistance biomarker is excluded from the methods of treatment using the mTOR inhibitor nanoparticles as described herein. In some embodiments, the status of the resistance biomarkers combined with the status of one or more of the mTOR-activating aberrations are used as the basis for selecting an individual for any one of the methods of treatment using mTOR inhibitor nanoparticles as described herein.

For example, TFE3, also known as transcription factor binding to IGHM enhancer 3, TFEA, RCCP2, RCCX1, or bHLHe33, is a transcription factor that specifically recognizes and binds MUE3-type E-box sequences in the promoters of genes. TFE3 promotes expression of genes downstream of transforming growth factor beta (TGF-beta) signaling. Translocation of TFE3 has been associated with renal cell carcinomas and other cancers. In some embodiments, the nucleic acid sequence of a wildtype TFE3 gene is identified by the Genbank accession number NC_000023.11 from nucleotide 49028726 to nucleotide 49043517 of the complement strand of chromosome X according to the GRCh38.p2 assembly of the human genome. Exemplary translocations of TFE3 that may be associated with resistance to treatment using the mTOR inhibitor nanoparticles as described herein include, but are not limited to, Xp11 translocation, such as t(X; 1)(p11.2; q21), t(X; 1)(p11.2; p34), (X; 17)(p11.2; q25.3), and inv(X)(p11.2; q12). Translocation of the TFE3 locus can be assessed using immunohistochemical methods or fluorescence in situ hybridization (FISH).

B. Based on Biomarker Indicative of Favorable Response to Treatment with an Anti-VEGF Antibody.

In some embodiments, there is provided a method of treating a colon cancer in an individual comprising administering to the individual a) an effective amount of a composition comprising nanoparticles comprising an mTOR inhibitor (such as a limus drug, e.g., sirolimus or a derivative thereof) and an albumin; b) an effective amount of anti-VEGF antibody, and c) a therapeutically effective FOLFOX regimen, wherein the individual is selected for treatment based on at least one biomarker indicative of favorable response to treatment with an anti-VEGF antibody. In some embodiments, the biomarker comprises an aberration in a gene that affects the response to treatment of a colon cancer in an individual with an anti-VEGF antibody (hereinafter also referred to as a “VEGF-associated gene”). In some embodiments, the at least one VEGF-associated biomarker comprises a mutation of a VEGF-associated gene. In some embodiments, the at least one VEGF-associated biomarker comprises a copy number variation of a VEGF-associated gene. In some embodiments, the at least one VEGF-associated biomarker comprises an aberrant expression level of a VEGF-associated gene. In some embodiments, the at least one VEGF-associated biomarker comprises an aberrant activity level of a VEGF-associated gene. In some embodiments, the at least one VEGF-associated biomarker comprises an aberrant phosphorylation level of the protein encoded by the VEGF-associated gene. In some embodiments, the VEGF-associated gene is selected from the group consisting of the genes encoding VEGF, VEGFR1, PIGF, lactate dehydrogenase (LDH) A, Glut1, HIF1α, IL-1β, IL-6, IL-8, IL-10, macrophage-derived chemokine, EGF, mismatch repair (MMR) protein, CCL18, cadherin 12 (CDH12), VE-cadherin, N-cadherin and Leucine-rich-alpha-2-glycoprotein 1 (LRG1). In some embodiments, the biomarker is selected from the group consisting of blood pressure, circulating VEGF, VEGF expression in cancer tissue, circulating PIGF, soluble VEGF receptors, intratumoral mRNA level of VEGFR1, lactate dehydrogenase (LDH) A, Glut1, or HIF1α, serum level of LDH, IL-1β, IL-6, IL-8, IL-10, macrophage-derived chemokine, or EGF, IL-8A-251T polymorphism, the number of circulating endothelial cells or bone marrow derived circulating endothelial cell progenitors, microvessel or vascular density (e.g., measured with CD31), endothelial signaling events (such as the ERK phosphorylation status and AKT phosphorylation status in tumor endothelial cells), microRNA-107, microRNA-145, microRNA-17-92, microRNA-194, mismatch repair (MMR) protein, infiltration of tumor-associated macrophages (TAM), CCL18, the mobilization of immune cells (such as MDSCs or TAMs), frequency of microsatellites, cadherin 12 (CDH12), VE-cadherin, N-cadherin and Leucine-rich-alpha-2-glycoprotein 1 (LRG1).

In some embodiments, there is provided a method of treating a colon cancer in an individual comprising: (a) assessing at least one VEGF-associated biomarker in the individual; and (b) administering to the individual i) an effective amount of a composition comprising nanoparticles comprising an mTOR inhibitor (such as a limus drug, e.g., sirolimus or a derivative thereof) and an albumin; ii) an effective amount of anti-VEGF antibody, and iii) a therapeutically effective FOLFOX regimen, wherein the individual is selected for treatment based on having at least one VEGF-associated biomarker.

In some embodiments, there is provided a method of treating a colon cancer in an individual comprising: (a) assessing at least one VEGF-associated biomarker in the individual; (b) selecting (e.g., identifying or recommending) the individual for treatment based on the individual having the at least one VEGF-associated biomarker; and (c) administering to the individual i) an effective amount of a composition comprising nanoparticles comprising an mTOR inhibitor (such as a limus drug, e.g., sirolimus or a derivative thereof) and an albumin; ii) an effective amount of anti-VEGF antibody, and iii) a therapeutically effective FOLFOX regimen.

In some embodiments, there is provided a method of selecting (including identifying or recommending) an individual having a colon cancer for treatment with i) an effective amount of a composition comprising nanoparticles comprising an mTOR inhibitor (such as a limus drug, e.g., sirolimus or a derivative thereof) and an albumin; ii) an effective amount of anti-VEGF antibody, and iii) a therapeutically effective FOLFOX regimen, wherein the method comprises (a) assessing at least one VEGF-associated biomarker in the individual; and (b) selecting or recommending the individual for treatment based on the individual having the at least one VEGF-associated biomarker.

In some embodiments, there is provided a method of selecting (including identifying or recommending) and treating an individual having a colon cancer, wherein the method comprises (a) assessing at least one VEGF-associated biomarker in the individual; (b) selecting or recommending the individual for treatment based on the individual having the at least one VEGF-associated biomarker; and (c) administering to the individual i) an effective amount of a composition comprising an mTOR inhibitor (such as a limus drug, e.g., sirolimus or a derivative thereof) and an albumin; ii) an effective amount of anti-VEGF antibody, and iii) a therapeutically effective FOLFOX regimen.

Also provided herein are methods of assessing whether an individual with a colon cancer is more likely to respond or less likely to respond to treatment based on the individual having at least one VEGF-associated biomarker, wherein the treatment comprises i) an effective amount of a composition comprising an mTOR inhibitor (such as a limus drug, e.g., sirolimus or a derivative thereof) and an albumin; ii) an effective amount of anti-VEGF antibody, and iii) a therapeutically effective FOLFOX regimen; the method comprising assessing at least one VEGF-associated biomarker in the individual. In some embodiments, the method further comprises administering to the individual who is determined to be likely to respond to the treatment i) an effective amount of a composition comprising an mTOR inhibitor (such as a limus drug, e.g., sirolimus or a derivative thereof) and an albumin; ii) an effective amount of anti-VEGF antibody, and iii) a therapeutically effective FOLFOX regimen. In some embodiments, the presence of the at least one VEGF-associated biomarker indicates that the individual is more likely to respond to the treatment, and the absence of the at least one VEGF-associated biomarker indicates that the individual is less likely to respond to the treatment. In some embodiments, the amount of the VEGF is determined based on the presence of the at least one VEGF-associated biomarker in the individual.

Also provided herein are methods of adjusting therapy treatment of an individual with a colon cancer receiving i) an effective amount of a composition comprising an mTOR inhibitor (such as a limus drug, e.g., sirolimus or a derivative thereof) and an albumin; ii) an effective amount of anti-VEGF antibody, and iii) a therapeutically effective FOLFOX regimen, the method comprising assessing at least one VEGF-associated biomarker in a sample isolated from the individual, and adjusting the therapy treatment based on the individual having the at least one VEGF-associated biomarker. In some embodiments, the amount of the anti-VEGF antibody is adjusted.

C. Based on Biomarker Indicative of Favorable Response to Treatment with a FOLFOX Regimen.

In some embodiments, there is provided a method of treating a colon cancer in an individual comprising administering to the individual a) an effective amount of a composition comprising nanoparticles comprising an mTOR inhibitor (such as a limus drug, e.g., sirolimus or a derivative thereof) and an albumin; b) an effective amount of anti-VEGF antibody, and c) a therapeutically effective FOLFOX regimen, wherein the individual is selected for treatment based on at least one biomarker indicative of favorable response to treatment with FOLFOX. In some embodiments, the biomarker comprises an aberration in a gene that affects the response to treatment of a colon cancer in an individual with FOLFOX (hereinafter also referred to as an “FOLFOX-associated gene”). In some embodiments, the at least one FOLFOX-associated biomarker comprises a mutation of a FOLFOX-associated gene. In some embodiments, the at least one FOLFOX-associated biomarker comprises a copy number variation of a FOLFOX-associated gene. In some embodiments, the at least one FOLFOX-associated biomarker comprises an aberrant expression level of a FOLFOX-associated gene. In some embodiments, the at least one FOLFOX-associated biomarker comprises an aberrant activity level of a FOLFOX-associated gene. In some embodiments, the at least one FOLFOX-associated biomarker comprises an aberrant phosphorylation level of the protein encoded by the FOLFOX-associated gene. In some embodiments, the FOLFOX-associated gene is selected from the group consisting of the genes encoding thymidylate synthase (TS), thymidine phosphorylase (TP), dihydropyrimidine dehydrogenase (DPD), UDP-glucuronosyltransferase 1A1 (UGT1A1) and excision repair cross-complementation group 1 (ERCC1). In some embodiments, the biomarker is selected from the group consisting of thymidylate synthase (TS) in tumor, polymorphism in the TS (e.g., polymorphis in TS promotor enhancer region (TSER, e.g., 3R and 2R variants), loss of heterozygosity (LOH) in the TS locus), thymidine phosphorylase (TP), dihydropyrimidine dehydrogenase (DPD), UDP-glucuronosyltransferase 1A1 (UGT1A1), UGT1A1 polymorphism (such as *28 or *6 polymorphism), the expression of excision repair cross-complementation group 1 (ERCC1), and ERCC1 polymorphism (such as ERCC1-118, XPD-751, XPG Arg1104His).

In some embodiments, there is provided a method of treating a colon cancer in an individual comprising: (a) assessing at least one FOLFOX-associated biomarker in the individual; and (b) administering to the individual i) an effective amount of a composition comprising nanoparticles comprising an mTOR inhibitor (such as a limus drug, e.g., sirolimus or a derivative thereof) and an albumin; ii) an effective amount of anti-VEGF antibody, and iii) a therapeutically effective FOLFOX regimen, wherein the individual is selected for treatment based on having at least one FOLFOX-associated biomarker.

In some embodiments, there is provided a method of treating a colon cancer in an individual comprising: (a) assessing at least one FOLFOX-associated biomarker in the individual; (b) selecting (e.g., identifying or recommending) the individual for treatment based on the individual having the at least one FOLFOX-associated biomarker; and (c) administering to the individual i) an effective amount of a composition comprising nanoparticles comprising an mTOR inhibitor (such as a limus drug, e.g., sirolimus or a derivative thereof) and an albumin; ii) an effective amount of anti-VEGF antibody, and iii) a therapeutically effective FOLFOX regimen.

In some embodiments, there is provided a method of selecting (including identifying or recommending) an individual having a colon cancer for treatment with i) an effective amount of a composition comprising nanoparticles comprising an mTOR inhibitor (such as a limus drug, e.g., sirolimus or a derivative thereof) and an albumin; ii) an effective amount of anti-VEGF antibody, and iii) a therapeutically effective FOLFOX regimen, wherein the method comprises (a) assessing at least one FOLFOX-associated biomarker in the individual; and (b) selecting or recommending the individual for treatment based on the individual having the at least one FOLFOX-associated biomarker.

In some embodiments, there is provided a method of selecting (including identifying or recommending) and treating an individual having a colon cancer, wherein the method comprises (a) assessing at least one FOLFOX-associated biomarker in the individual; (b) selecting or recommending the individual for treatment based on the individual having the at least one FOLFOX-associated biomarker; and (c) administering to the individual i) an effective amount of a composition comprising an mTOR inhibitor (such as a limus drug, e.g., sirolimus or a derivative thereof) and an albumin; ii) an effective amount of anti-VEGF antibody, and iii) a therapeutically effective FOLFOX regimen.

Also provided herein are methods of assessing whether an individual with a colon cancer is more likely to respond or less likely to respond to treatment based on the individual having at least one FOLFOX-associated biomarker, wherein the treatment comprises i) an effective amount of a composition comprising an mTOR inhibitor (such as a limus drug, e.g., sirolimus or a derivative thereof) and an albumin; ii) an effective amount of anti-VEGF antibody, and iii) a therapeutically effective FOLFOX regimen; the method comprising assessing at least one FOLFOX-associated biomarker in the individual. In some embodiments, the method further comprises administering to the individual who is determined to be likely to respond to the treatment i) an effective amount of a composition comprising an mTOR inhibitor (such as a limus drug, e.g., sirolimus or a derivative thereof) and an albumin; ii) an effective amount of anti-VEGF antibody, and iii) a therapeutically effective FOLFOX regimen. In some embodiments, the presence of the at least one FOLFOX-associated biomarker indicates that the individual is more likely to respond to the treatment, and the absence of the at least one FOLFOX-associated biomarker indicates that the individual is less likely to respond to the treatment. In some embodiments, the FOLFOX regimen is determined based on the presence of the at least one FOLFOX-associated biomarker in the individual.

Also provided herein are methods of adjusting therapy treatment of an individual with a colon cancer receiving i) an effective amount of a composition comprising an mTOR inhibitor (such as a limus drug, e.g., sirolimus or a derivative thereof) and an albumin; ii) an effective amount of anti-VEGF antibody, and iii) a therapeutically effective FOLFOX regimen, the method comprising assessing at least one FOLFOX-associated biomarker in a sample isolated from the individual, and adjusting the therapy treatment based on the individual having the at least one FOLFOX-associated biomarker. In some embodiments, the FOLFOX regimen is modified.

Further contemplated are combinations of the methods described in this section, such that treatment of an individual may depend on the presence of an mTOR-activating aberration and any of the VEGF and FOLFOX-associated biomarkers described herein.

### Nanoparticle Compositions

The mTOR inhibitor nanoparticle compositions described herein comprise nanoparticles comprising (in various embodiments consisting essentially of or consisting of) an mTOR inhibitor (such as a limus drug, e.g., sirolimus or a derivative thereof) and an albumin (such as human serum albumin). Nanoparticles of poorly water soluble drugs (such as macrolides) have been disclosed in, for example, U.S. Pat. Nos. 5,916,596; 6,506,405; 6,749,868, 6,537,579, 7,820,788, and 8,911,786, and also in U. S. Pat. Pub. Nos. 2006/0263434, and 2007/0082838; PCT Patent Application WO08/137148, each of which is incorporated herein by reference in their entirety.

In some embodiments, the composition comprises nanoparticles with an average or mean diameter of no greater than about 1000 nanometers (nm), such as no greater than about any of 900, 800, 700, 600, 500, 400, 300, 200, and 100 nm. In some embodiments, the average or mean diameters of the nanoparticles is no greater than about 200 nm. In some embodiments, the average or mean diameters of the nanoparticles is no greater than about 150 nm. In some embodiments, the average or mean diameters of the nanoparticles is no greater than about 100 nm. In some embodiments, the average or mean diameter of the nanoparticles is about 10 to about 400 nm. In some embodiments, the average or mean diameter of the nanoparticles is about 10 to about 150 nm. In some embodiments, the average or mean diameter of the nanoparticles is about 40 to about 120 nm. In some embodiments, the average or mean diameter of the nanoparticles are no less than about 50 nm. In some embodiments, the nanoparticles are sterile-filterable.

In some embodiments, the nanoparticles in the composition described herein have an average diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least about any one of 60%, 70%, 80%, 90%, 95%, or 99%) of the nanoparticles in the composition have a diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least any one of 60%, 70%, 80%, 90%, 95%, or 99%) of the nanoparticles in the composition fall within the range of about 10 nm to about 400 nm, including for example about 10 nm to about 200 nm, about 20 nm to about 200 nm, about 30 nm to about 180 nm, about 40 nm to about 150 nm, about 40 nm to about 120 nm, and about 60 nm to about 100 nm.

In some embodiments, the albumin has sulfhydryl groups that can form disulfide bonds. In some embodiments, at least about 5% (including for example at least about any one of 10%, 15%, 20%, 25%, 30%, 40%, 50%, 60%, 70%, 80%, or 90%) of the albumin in the nanoparticle portion of the composition are crosslinked (for example crosslinked through one or more disulfide bonds).

In some embodiments, the nanoparticles comprising the mTOR inhibitor (such as a limus drug, e.g., sirolimus or a derivative thereof) are associated (e.g., coated) with an albumin (such as human albumin or human serum albumin). In some embodiments, the composition comprises an mTOR inhibitor (such as a limus drug, e.g., sirolimus or a derivative thereof) in both nanoparticle and non-nanoparticle forms (e.g., in the form of solutions or in the form of soluble albumin/nanoparticle complexes), wherein at least about any one of 50%, 60%, 70%, 80%, 90%, 95%, or 99% of the mTOR inhibitor in the composition are in nanoparticle form. In some embodiments, the mTOR inhibitor (such as a limus drug, e.g., sirolimus or a derivative thereof) in the nanoparticles constitutes more than about any one of 50%, 60%, 70%, 80%, 90%, 95%, or 99% of the nanoparticles by weight. In some embodiments, the nanoparticles have a non-polymeric matrix. In some embodiments, the nanoparticles comprise a core of an mTOR inhibitor (such as a limus drug, e.g., sirolimus or a derivative thereof) that is substantially free of polymeric materials (such as polymeric matrix).

In some embodiments, the composition comprises an albumin in both nanoparticle and non-nanoparticle portions of the composition, wherein at least about any one of 50%, 60%, 70%, 80%, 90%, 95%, or 99% of the albumin in the composition are in non-nanoparticle portion of the composition.

In some embodiments, the weight ratio of an albumin (such as human albumin or human serum albumin) and a mTOR inhibitor (such as a limus drug, e.g., sirolimus or a derivative thereof) in the mTOR inhibitor nanoparticle composition is about 18:1 or less, such as about 15:1 or less, for example about 10:1 or less. In some embodiments, the weight ratio of an albumin (such as human albumin or human serum albumin) and an mTOR inhibitor (such as a limus drug, e.g., sirolimus or a derivative thereof) in the composition falls within the range of any one of about 1:1 to about 18:1, about 2:1 to about 15:1, about 3:1 to about 13:1, about 4:1 to about 12:1, about 5:1 to about 10:1. In some embodiments, the weight ratio of an albumin and an mTOR inhibitor (such as a limus drug, e.g., sirolimus or a derivative thereof) in the nanoparticle portion of the composition is about any one of 1:2, 1:3, 1:4, 1:5, 1:9, 1:10, 1:15, or less. In some embodiments, the weight ratio of the albumin (such as human albumin or human serum albumin) and the mTOR inhibitor (such as a limus drug, e.g., sirolimus or a derivative thereof) in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the mTOR inhibitor nanoparticle composition (such as sirolimus/albumin nanoparticle composition) comprises one or more of the above characteristics.

The nanoparticles described herein may be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

In some embodiments, the pharmaceutically acceptable carrier comprises an albumin (such as human albumin or human serum albumin). The albumin may either be natural in origin or synthetically prepared. In some embodiments, the albumin is human albumin or human serum albumin. In some embodiments, the albumin is a recombinant albumin.

Human serum albumin (HSA) is a highly soluble globular protein of Mᵣ 65K and consists of 585 amino acids. HSA is the most abundant protein in the plasma and accounts for 70-80% of the colloid osmotic pressure of human plasma. The amino acid sequence of HSA contains a total of 17 disulfide bridges, one free thiol (Cys 34), and a single tryptophan (Trp 214). Intravenous use of HSA solution has been indicated for the prevention and treatment of hypovolemic shock (see, e.g., Tullis, JAMA, 237: 355-360, 460-463, (1977)) and Houser et al., Surgery, Gynecology and Obstetrics, 150: 811-816 (1980)) and in conjunction with exchange transfusion in the treatment of neonatal hyperbilirubinemia (see, e.g., Finlayson, Seminars in Thrombosis and Hemostasis, 6, 85-120, (1980)). Other albumins are contemplated, such as bovine serum albumin. Use of such non-human albumins could be appropriate, for example, in the context of use of these compositions in non-human mammals, such as the veterinary (including domestic pets and agricultural context). Human serum albumin (HSA) has multiple hydrophobic binding sites (a total of eight for fatty acids, an endogenous ligand of HSA) and binds a diverse set of drugs, especially neutral and negatively charged hydrophobic compounds (Goodman et al., The Pharmacological Basis of Therapeutics, 9^{th} ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (198a), Vorum, Dan. Med. Bull., 46, 379-99 (1999), Kragh-Hansen, Dan. Med. Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (199b), and Carter et al., Adv. Protein. Chem., 45, 153-203 (1994)). Rapamycin and propofol have been shown to bind HSA (see, e.g., Paal et al., Eur. J. Biochem., 268(7), 2187-91 (200a), Purcell et al., Biochim. Biophys. Acta, 1478(a), 61-8 (2000), Altmayer et al., Arzneimittelforschung, 45, 1053-6 (1995), and Garrido et al., Rev. Esp. Anestestiol. Reanim., 41, 308-12 (1994)). In addition, docetaxel has been shown to bind to human plasma proteins (see, e.g., Urien et al., Invest. New Drugs, 14(b), 147-51 (1996)).

In some embodiments, the composition described herein is substantially free (such as free) of surfactants, such as Cremophor (or polyoxyethylated castor oil, including Cremophor EL® (BASF)). In some embodiments, the mTOR inhibitor nanoparticle composition (such as sirolimus/albumin nanoparticle composition) is substantially free (such as free) of surfactants. A composition is “substantially free of Cremophor” or “substantially free of surfactant” if the amount of Cremophor or surfactant in the composition is not sufficient to cause one or more side effect(s) in an individual when the mTOR inhibitor nanoparticle composition (such as sirolimus/albumin nanoparticle composition) is administered to the individual. In some embodiments, the mTOR inhibitor nanoparticle composition (such as sirolimus/albumin nanoparticle composition) contains less than about any one of 20%, 15%, 10%, 7.5%, 5%, 2.5%, or 1% organic solvent or surfactant. In some embodiments, the albumin is human albumin or human serum albumin. In some embodiments, the albumin is recombinant albumin.

The amount of an albumin in the composition described herein will vary depending on other components in the composition. In some embodiments, the composition comprises an albumin in an amount that is sufficient to stabilize the mTOR inhibitor (such as a limus drug, e.g., sirolimus or a derivative thereof) in an aqueous suspension, for example, in the form of a stable colloidal suspension (such as a stable suspension of nanoparticles). In some embodiments, the albumin is in an amount that reduces the sedimentation rate of the mTOR inhibitor (such as a limus drug, e.g., sirolimus or a derivative thereof) in an aqueous medium. For particle-containing compositions, the amount of the albumin also depends on the size and density of nanoparticles of the mTOR inhibitor.

An mTOR inhibitor (such as a limus drug, e.g., sirolimus or a derivative thereof) is “stabilized” in an aqueous suspension if it remains suspended in an aqueous medium (such as without visible precipitation or sedimentation) for an extended period of time, such as for at least about any of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to an individual (such as a human). Stability of the suspension is generally (but not necessarily) evaluated at a storage temperature (such as room temperature (such as 20-25° C.) or refrigerated conditions (such as 4° C.)). For example, a suspension is stable at a storage temperature if it exhibits no flocculation or particle agglomeration visible to the naked eye or when viewed using an optical microscope at 1000 times, at about fifteen minutes after preparation of the suspension. Stability can also be evaluated under accelerated testing conditions, such as at a temperature that is about 40° C. or higher.

In some embodiments, the albumin is present in an amount that is sufficient to stabilize the mTOR inhibitor (such as a limus drug, e.g., sirolimus or a derivative thereof) in an aqueous suspension at a certain concentration. For example, the concentration of the mTOR inhibitor (such as a limus drug, e.g., sirolimus or a derivative thereof) in the composition is about 0.1 to about 100 mg/ml, including for example about any of 0.1 to about 50 mg/ml, about 0.1 to about 20 mg/ml, about 1 to about 10 mg/ml, about 2 mg/ml to about 8 mg/ml, about 4 to about 6 mg/ml, or about 5 mg/ml. In some embodiments, the concentration of the mTOR inhibitor (such as a limus drug, e.g., sirolimus or a derivative thereof) is at least about any of 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the albumin is present in an amount that avoids use of surfactants (such as Cremophor), so that the composition is free or substantially free of surfactant (such as Cremophor).

In some embodiments, the composition, in liquid form, comprises from about 0.1% to about 50% (w/v) (e.g., about 0.5% (w/v), about 5% (w/v), about 10% (w/v), about 15% (w/v), about 20% (w/v), about 30% (w/v), about 40% (w/v), or about 50% (w/v)) of an albumin. In some embodiments, the composition, in liquid form, comprises about 0.5% to about 5% (w/v) of albumin.

In some embodiments, the weight ratio of the albumin to the mTOR inhibitor (such as a limus drug, e.g., sirolimus or a derivative thereof) in the mTOR inhibitor nanoparticle composition is such that a sufficient amount of mTOR inhibitor binds to, or is transported by, the cell. While the weight ratio of an albumin to an mTOR inhibitor (such as a limus drug, e.g., sirolimus or a derivative thereof) will have to be optimized for different albumin and mTOR inhibitor combinations, generally the weight ratio of an albumin to an mTOR inhibitor (such as a limus drug, e.g., sirolimus or a derivative thereof) (w/w) is about 0.01:1 to about 100:1, about 0.02:1 to about 50:1, about 0.05:1 to about 20:1, about 0.1:1 to about 20:1, about 1:1 to about 18:1, about 2:1 to about 15:1, about 3:1 to about 12:1, about 4:1 to about 10:1, about 5:1 to about 9:1, or about 9:1. In some embodiments, the albumin to mTOR inhibitor (such as a limus drug, e.g., sirolimus or a derivative thereof) weight ratio is about any of 18:1 or less, 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, and 3:1 or less. In some embodiments, the weight ratio of the albumin (such as human albumin or human serum albumin) to the mTOR inhibitor (such as a limus drug, e.g., sirolimus or a derivative thereof) in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the albumin allows the composition to be administered to an individual (such as a human) without significant side effects. In some embodiments, the albumin (such as human serum albumin or human albumin) is in an amount that is effective to reduce one or more side effects of administration of the mTOR inhibitor (such as a limus drug, e.g., sirolimus or a derivative thereof) to a human. The term “reducing one or more side effects” of administration of the mTOR inhibitor (such as a limus drug, e.g., sirolimus or a derivative thereof) refers to reduction, alleviation, elimination, or avoidance of one or more undesirable effects caused by the mTOR inhibitor, as well as side effects caused by delivery vehicles (such as solvents that render the limus drugs suitable for injection) used to deliver the mTOR inhibitor. Such side effects include, for example, myelosuppression, neurotoxicity, hypersensitivity, inflammation, venous irritation, phlebitis, pain, skin irritation, peripheral neuropathy, neutropenic fever, anaphylactic reaction, venous thrombosis, extravasation, and combinations thereof. These side effects, however, are merely exemplary and other side effects, or combination of side effects, associated with limus drugs (such as a limus drug, e.g., sirolimus or a derivative thereof) can be reduced.

In some embodiments, the mTOR inhibitor nanoparticle compositions described herein comprise nanoparticles comprising an mTOR inhibitor (such as a limus drug, e.g., sirolimus or a derivative thereof) and an albumin (such as human albumin or human serum albumin), wherein the nanoparticles have an average diameter of no greater than about 200 nm. In some embodiments, the mTOR inhibitor nanoparticle compositions described herein comprise nanoparticles comprising an mTOR inhibitor (such as a limus drug, e.g., sirolimus or a derivative thereof) and an albumin (such as human albumin or human serum albumin), wherein the nanoparticles have an average diameter of no greater than about 150 nm. In some embodiments, the mTOR inhibitor nanoparticle compositions described herein comprise nanoparticles comprising an mTOR inhibitor (such as a limus drug, e.g., sirolimus or a derivative thereof) and an albumin (such as human albumin or human serum albumin), wherein the nanoparticles have an average diameter of no greater than about 150 nm (for example about 100 nm). In some embodiments, the mTOR inhibitor nanoparticle compositions described herein comprise nanoparticles comprising sirolimus and human albumin (such as human serum albumin), wherein the nanoparticles have an average diameter of no greater than about 150 nm (for example about 100 nm). In some embodiments, the mTOR inhibitor nanoparticle compositions described herein comprise nanoparticles comprising sirolimus and human albumin (such as human serum albumin), wherein the average or mean diameter of the nanoparticles is about 10 to about 150 nm. In some embodiments, the mTOR inhibitor nanoparticle compositions described herein comprise nanoparticles comprising sirolimus and human albumin (such as human serum albumin), wherein the average or mean diameter of the nanoparticles is about 40 to about 120 nm.

In some embodiments, the mTOR inhibitor nanoparticle compositions described herein comprise nanoparticles comprising an mTOR inhibitor (such as a limus drug, e.g., sirolimus or a derivative thereof) and an albumin (such as human albumin or human serum albumin), wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) and an albumin (such as human albumin or human serum albumin), wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) and an albumin (such as human albumin or human serum albumin), wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising sirolimus and human albumin (such as human serum albumin), wherein the nanoparticles have an average diameter of no greater than about 150 nm (for example about 100 nm), wherein the weight ratio of albumin and mTOR inhibitor in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm.

In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin). In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of no greater than about 200 nm. In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of no greater than about 150 nm. In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of about 10 nm to about 150 nm. In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of about 40 nm to about 120 nm. In some embodiments, the mTOR inhibitor nanoparticle compositions described herein comprise nanoparticles comprising sirolimus associated (e.g., coated) with human albumin (such as human serum albumin), wherein the nanoparticles have an average diameter of no greater than about 150 nm (for example about 100 nm). In some embodiments, the mTOR inhibitor nanoparticle compositions described herein comprise nanoparticles comprising sirolimus associated (e.g., coated) with human albumin (such as human serum albumin), wherein the nanoparticles have an average diameter of about 10 nm to about 150 nm. In some embodiments, the mTOR inhibitor nanoparticle compositions described herein comprise nanoparticles comprising sirolimus associated (e.g., coated) with human albumin (such as human serum albumin), wherein the nanoparticles have an average diameter of about 40 nm to about 120 nm.

In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising sirolimus associated (e.g., coated) with human albumin (such as human serum albumin), wherein the nanoparticles have an average diameter of no greater than about 150 nm (for example about 100 nm), wherein the weight ratio of albumin and the sirolimus in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm.

In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin). In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the nanoparticles have an average diameter of no greater than about 200 nm. In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the nanoparticles have an average diameter of no greater than about 150 nm. In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the nanoparticles have an average diameter of no greater than about 150 nm (for example about 100 nm). In some embodiments, the mTOR inhibitor nanoparticle compositions described herein comprise nanoparticles comprising sirolimus stabilized by human albumin (such as human serum albumin), wherein the nanoparticles have an average diameter of no greater than about 150 nm (for example about 100 nm). In some embodiments, the average or mean diameter of the nanoparticles is about 10 nm to about 150 nm. In some embodiments, the average or mean diameter of the nanoparticles is about 40 nm to about 120 nm.

In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising sirolimus stabilized by human albumin (such as human serum albumin), wherein the nanoparticles have an average diameter of no greater than about 150 nm (for example about 100 nm), wherein the weight ratio of albumin and the sirolimus in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm.

In some embodiments, the mTOR inhibitor nanoparticle composition comprises nab-sirolimus. In some embodiments, the mTOR inhibitor nanoparticle composition is nab-sirolimus. Nab-sirolimus is a formulation of sirolimus stabilized by human albumin USP, which can be dispersed in directly injectable physiological solution. The weight ratio of human albumin and sirolimus is about 8:1 to about 9:1. When dispersed in a suitable aqueous medium such as 0.9% sodium chloride injection or 5% dextrose injection, nab-sirolimus forms a stable colloidal suspension of sirolimus. The mean particle size of the nanoparticles in the colloidal suspension is about 100 nanometers. Since HSA is freely soluble in water, nab-sirolimus can be reconstituted in a wide range of concentrations ranging from dilute (0.1 mg/ml sirolimus or a derivative thereof) to concentrated (20 mg/ml sirolimus or a derivative thereof), including for example about 2 mg/ml to about 8 mg/ml, or about 5 mg/ml.

Methods of making nanoparticle compositions are known in the art. For example, nanoparticles containing an mTOR inhibitor (such as a limus drug, e.g., sirolimus or a derivative thereof) and an albumin (such as human serum albumin or human albumin) can be prepared under conditions of high shear forces (e.g., sonication, high pressure homogenization, or the like). These methods are disclosed in, for example, U.S. Pat. Nos. 5,916,596; 6,506,405; 6,749,868, 6,537,579, 7,820,788, and 8,911,786, and also in U. S. Pat. Pub. Nos. 2007/0082838, 2006/0263434 and PCT Application WO08/137148.

Briefly, the mTOR inhibitor (such as a limus drug, e.g., sirolimus or a derivative thereof) is dissolved in an organic solvent, and the solution can be added to an albumin solution. The mixture is subjected to high pressure homogenization. The organic solvent can then be removed by evaporation. The dispersion obtained can be further lyophilized. Suitable organic solvent include, for example, ketones, esters, ethers, chlorinated solvents, and other solvents known in the art. For example, the organic solvent can be methylene chloride or chloroform/ethanol (for example with a ratio of 1:9, 1:8, 1:7, 1:6, 1:5, 1:4, 1:3, 1:2, 1:1, 2:1, 3:1, 4:1, 5:1, 6:1, 7:1, 8:1, or 9:1).

mTOR Inhibitor

The methods described herein in some embodiments comprise administration of nanoparticle compositions of mTOR inhibitors. “mTOR inhibitor” used herein refers to an inhibitor of mTOR. mTOR is a serine/threonine-specific protein kinase downstream of the phosphatidylinositol 3-kinase (PI3K)/Akt (protein kinase B) pathway, and a key regulator of cell survival, proliferation, stress, and metabolism. mTOR pathway dysregulation has been found in many human carcinomas, and mTOR inhibition produced substantial inhibitory effects on tumor progression.

The mammalian target of rapamycin (mTOR) (also known as mechanistic target of rapamycin or FK506 binding protein 12-rapamycin associated protein 1 (FRAP1)) is an atypical serine/threonine protein kinase that is present in two distinct complexes, mTOR Complex 1 (mTORC1) and mTOR Complex 2 (mTORC2). mTORC1 is composed of mTOR, regulatory-associated protein of mTOR (Raptor), mammalian lethal with SEC13 protein 8 (MLST8), PRAS40 and DEPTOR (Kim et al. (2002). Cell 110: 163-75; Fang et al. (2001). Science 294 (5548): 1942-5). mTORC1 integrates four major signal inputs: nutrients (such as amino acids and phosphatidic acid), growth factors (insulin), energy and stress (such as hypoxia and DNA damage). Amino acid availability is signaled to mTORC1 via a pathway involving the Rag and Regulator (LAMTOR1-3) Growth factors and hormones (e.g., insulin) signal to mTORC1 via Akt, which inactivates TSC2 to prevent inhibition of mTORC1. Alternatively, low ATP levels lead to the AMPK-dependent activation of TSC2 and phosphorylation of raptor to reduce mTORC1 signaling proteins.

Active mTORC1 has a number of downstream biological effects including translation of mRNA via the phosphorylation of downstream targets (4E-BP1 and p70 S6 Kinase), suppression of autophagy (Atg13, ULK1), ribosome biogenesis, and activation of transcription leading to mitochondrial metabolism or adipogenesis. Accordingly, mTORC1 activity promotes either cellular growth when conditions are favorable or catabolic processes during stress or when conditions are unfavorable.

mTORC2 is composed of mTOR, rapamycin-insensitive companion of mTOR (RICTOR), GβL, and mammalian stress-activated protein kinase interacting protein 1 (mSIN1). In contrast to mTORC1, for which many upstream signals and cellular functions have been defined (see above), relatively little is known about mTORC2 biology. mTORC2 regulates cytoskeletal organization through its stimulation of F-actin stress fibers, paxillin, RhoA, Rac1, Cdc42, and protein kinase C α (PKCα). It had been observed that knocking down mTORC2 components affects actin polymerization and perturbs cell morphology (Jacinto et al. (2004). Nat. Cell Biol. 6, 1122-1128; Sarbassov et al. (2004). Curr. Biol. 14, 1296-1302). This suggests that mTORC2 controls the actin cytoskeleton by promoting protein kinase Cα (PKCα) phosphorylation, phosphorylation of paxillin and its relocalization to focal adhesions, and the GTP loading of RhoA and Rac1. The molecular mechanism by which mTORC2 regulates these processes has not been determined.

In some embodiments, the mTOR inhibitor (such as a limus drug, e.g., sirolimus or a derivative thereof) is an inhibitor of mTORC1. In some embodiments, the mTOR inhibitor (such as a limus drug, e.g., sirolimus or a derivative thereof) is an inhibitor of mTORC2. In some embodiments, the mTOR inhibitor (such as a limus drug, e.g., sirolimus or a derivative thereof) is an inhibitor of both mTORC1 and mTORC2.

In some embodiments, the mTOR inhibitor is a limus drug, which includes sirolimus and its analogs. Examples of limus drugs include, but are not limited to, temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the limus drug is selected from the group consisting of temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the mTOR inhibitor is an mTOR kinase inhibitor, such as CC-115 or CC-223.

In some embodiments, the mTOR inhibitor is sirolimus. Sirolimus is macrolide antibiotic that complexes with FKBP-12 and inhibits the mTOR pathway by binding mTORC1.

In some embodiments, the mTOR inhibitor is selected from the group consisting of sirolimus (rapamycin), BEZ235 (NVP-BEZ235), everolimus (also known as RAD001, Zortress, Certican, and Afinitor), AZD8055,temsirolimus (also known as CCI-779 and Torisel), CC-115, CC-223, PI-103, Ku-0063794, INK 128, AZD2014, NVP-BGT226, PF-04691502, CH5132799, GDC-0980 (RG7422), Torin 1, WAY-600, WYE-125132, WYE-687, GSK2126458, PF-05212384 (PKI-587), PP-121, OSI-027, Palomid 529, PP242, XL765, GSK1059615, WYE-354, and ridaforolimus (also known as deforolimus).

BEZ235 (NVP-BEZ235) is an imidazoquilonine derivative that is an mTORC1 catalytic inhibitor (Roper J, et al. PLoS One, 2011, 6(9), e25132). Everolimus is the 40-O-(2-hydroxyethyl) derivative of sirolimus and binds the cyclophilin FKBP-12, and this complex also mTORC1. AZD8055 is a small molecule that inhibits the phosphorylation of mTORC1 (p70S6K and 4E-BP1). Temsirolimus is a small molecule that forms a complex with the FK506-binding protein and prohibits the activation of mTOR when it resides in the mTORC1 complex. PI-103 is a small molecule that inhibits the activation of the rapamycin-sensitive (mTORC1) complex (Knight et al. (2006) Cell. 125: 733-47). KU-0063794 is a small molecule that inhibits the phosphorylation of mTORC1 at Ser2448 in a dose-dependent and time-dependent manner. INK 128, AZD2014, NVP-BGT226, CH5132799, WYE-687, and are each small molecule inhibitors of mTORC1. PF-04691502 inhibits mTORC1 activity. GDC-0980 is an orally bioavailable small molecule that inhibits Class I PI3 Kinase and TORC1. Torin 1 is a potent small molecule inhibitor of mTOR. WAY-600 is a potent, ATP-competitive and selective inhibitor of mTOR. WYE-125132 is an ATP-competitive small molecule inhibitor of mTORC1. GSK2126458 is an inhibitor of mTORC1. PKI-587 is a highly potent dual inhibitor of PI3Kα, PI3Kγ and mTOR. PP-121 is a multi-target inhibitor of PDGFR, Hck, mTOR, VEGFR2, Src and Abl. OSI-027 is a selective and potent dual inhibitor of mTORC1 and mTORC2 with IC50 of 22 nM and 65 nM, respectively. Palomid 529 is a small molecule inhibitor of mTORC1 that lacks affinity for ABCB1/ABCG2 and has good brain penetration (Lin et al. (2013) Int J Cancer DOI: 10.1002/ijc. 28126 (e-published ahead of print). PP242 is a selective mTOR inhibitor. XL765 is a dual inhibitor of mTOR/PI3k for mTOR, p110α, p110β, p110γ and p110δ. GSK1059615 is a novel and dual inhibitor of PI3Kα, PI3Kβ, PI3Kδ, PI3Kγ and mTOR. WYE-354 inhibits mTORC1 in HEK293 cells (0.2 μM-5 μM) and in HUVEC cells (10 nM-1 μM). WYE-354 is a potent, specific and ATP-competitive inhibitor of mTOR. Deforolimus (Ridaforolimus, AP23573, MK-8669) is a selective mTOR inhibitor.

Other Components in the mTOR Inhibitor Nanoparticle Compositions

The nanoparticles described herein can be present in a composition that includes other agents, excipients, or stabilizers. For example, to increase stability by increasing the negative zeta potential of nanoparticles, certain negatively charged components may be added. Such negatively charged components include, but are not limited to bile salts of bile acids consisting of glycocholic acid, cholic acid, chenodeoxycholic acid, taurocholic acid, glycochenodeoxycholic acid, taurochenodeoxycholic acid, lithocholic acid, ursodeoxycholic acid, dehydrocholic acid and others; phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine stearoyloleoylphosphatidylcholine, stearoylarachidoylphosphatidylcholine, and dipalmitoylphosphatidylcholine. Other phospholipids including L-α-dimyristoylphosphatidylcholine (DMPC), dioleoylphosphatidylcholine (DOPC), distearyolphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

In some embodiments, the composition is suitable for administration to a human. In some embodiments, the composition is suitable for administration to a mammal such as, in the veterinary context, domestic pets and agricultural animals. There are a wide variety of suitable formulations of the mTOR inhibitor nanoparticle composition (such as sirolimus/albumin nanoparticle composition) (see, e.g., U.S. Pat. Nos. 5,916,596 and 6,096,331). The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Injectable formulations are preferred.

In some embodiments, the composition is formulated to have a pH range of about 4.5 to about 9.0, including for example pH ranges of about any of 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

### Anti-VEGF Antibody

Angiogenesis is an important cellular event in which vascular endothelial cells proliferate, prune and reorganize to form new vessels from preexisting vascular network. Angiogenesis is also implicated in the pathogenesis of a variety of disorders, including but not limited to, tumors, proliferative retinopathies, age-related macular degeneration, rheumatoid arthritis (RA), and psoriasis. Angiogenesis is essential for the growth of most primary tumors and their subsequent metastasis. Tumors can absorb sufficient nutrients and oxygen by simple diffusion up to a size of 1-2 mm, at which point their further growth requires the elaboration of vascular supply. This process is thought to involve recruitment of the neighboring host mature vasculature to begin sprouting new blood vessel capillaries, which grow towards, and subsequently infiltrate, the tumor mass. In addition, tumor angiogenesis involve the recruitment of circulating endothelial precursor cells from the bone marrow to promote neovascularization. Kerbel (2000) Carcinogenesis 21:505-515; Lynden et al. (2001) Nat. Med. 7:1194-1201.

Vascular endothelial cell growth factor (VEGF), which is also termed VEGF-A or vascular permeability factor (VPF), is a pivotal regulator of both normal and abnormal angiogenesis. Ferrara and Davis-Smyth (1997) Endocrine Rev. 18:4-25; Ferrara (1999) J. Mol. Med. 77:527-543.

The terms “VEGF” and “VEGF-A” are used interchangeably to refer to the 165-amino acid vascular endothelial cell growth factor and related 121-, 189-, and 206-amino acid vascular endothelial cell growth factors, as described by Leung et al. Science, 246:1306 (1989), and Houck et al. Mol. Endocrin., 5:1806 (1991), together with the naturally occurring allelic and processed forms thereof. In some embodiments, the term “VEGF” is also used to refer to truncated forms of the polypeptide comprising amino acids 8 to 109 or 1 to 109 of the 165-amino acid human vascular endothelial cell growth factor. The amino acid positions for a “truncated” native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native VEGF.

The methods described herein in some embodiments comprise administration of an anti-VEGF antibody. An “anti-VEGF antibody” is an antibody that binds to VEGF with sufficient affinity and specificity. In some embodiments, the anti-VEGF antibody is used as a therapeutic agent in targeting and interfering with diseases or conditions wherein the VEGF activity is involved. An anti-VEGF antibody will usually not bind to other VEGF homologues such as VEGF-B or VEGF-C, nor other growth factors such as P1GF, PDGF or bFGF. In some embodiments, the anti-VEGF antibody is a monoclonal antibody. In some embodiments, the anti-VEGF antibody binds to the same epitope as the monoclonal anti-VEGF antibody A4.6.1 produced by hybridoma ATCC HB 10709. In some embodiments, the anti-VEGF antibody is a recombinant antibody. In some embodiments, the anti-VEGF antibody is a humanized antibody. In some embodiments, the anti-VEGF is a recombinant humanized antibody. In some embodiments, the recombinant humanized anti-VEGF antibody is an antibody generated according to Presta et al. (1997) Cancer Res. 57:4593-4599, including but not limited to the antibody known as bevacizumab (BV; Avastin™).

In some embodiments, the anti-VEGF antibody is a fragment of an anti-VEGF antibody (e.g., a Fab fragment). In some embodiments, the anti-VEGF antibody is Ranibizumab.

### FOLFOX

The term “FOLFOX” as used herein refers to a combination therapy (e.g., chemotherapy) comprising at least one oxaliplatin compound chosen from oxaliplatin, pharmaceutically acceptable salts thereof, and solvates of any of the foregoing; at least one 5-fluorouracil (also known as 5-FU) compound chosen from 5-fluorouracil, pharmaceutically acceptable salts thereof, and solvates of any of the foregoing; and at least one folinic acid compound chosen from folinic acid (also known as leucovorin), levofolinate (the levo isoform of folinic acid), pharmaceutically acceptable salts of any of the foregoing, and solvates of any of the foregoing. The term “FOLFOX” as used herein is not intended to be limited to any particular amounts or dosing regimens for those components. Rather, as used herein, “FOLFOX” includes all combinations of those components in any amounts and dosing regimens. As used herein, any recitation of the term “FOLFOX” may be replaced with a recitation of the individual components. For example, the term “FOLFOX” may be replaced with the phrase “at least one oxaliplatin compound chosen from oxaliplatin, pharmaceutically acceptable salts of oxaliplatin, solvates of oxaliplatin, and solvates of pharmaceutically acceptable salts of oxaliplatin; at least one 5-fluorouracil compound chosen from 5-fluorouracil, pharmaceutically acceptable salts of 5-fluorouracil, solvates of 5-fluorouracil, and solvates of pharmaceutically acceptable salts of 5-fluorouracil; and at least one folinic acid compound chosen from leucovorin, levofolinate, pharmaceutically acceptable salts of any of the foregoing, and solvates of any of the foregoing.”

A “therapeutically effective FOLFOX regimen”, as used herein, means a therapeutically effective amount of the components of FOLFOX as defined herein administered according to a dosing regimen that is sufficient to effect the intended result including, but not limited to, disease treatment, as illustrated below. In some embodiments, a therapeutically effective regimen of FOLFOX comprises administering oxaliplatin together with leucovorin intravenously, followed by 5-FU intravenously. In some embodiments, a therapeutically effective FOLFOX regimen comprises administering oxaliplatin in the amount of from about 50 mg/m² to about 200 mg/m² together with leucovorin in the amount of from about 200 mg/m² to about 600 mg/m² intravenously, followed by 5-FU in the amount of from about 1200 mg/m² to about 3600 mg/m² intravenously. In some embodiments, a therapeutically effective FOLFOX regimen comprises administering oxaliplatin of about 85 mg/m² together with leucovorin of about 400 mg/m² intravenously, followed by 5-FU of about 2400 mg/m². In some embodiments, a therapeutically effective FOLFOX regimen comprises administering oxaliplatin of about 85 mg/m² together with leucovorin of about 400 mg/m² intravenously, followed by 5-FU of about 400 mg/m² bolus and 5-FU of about 1200 mg/m²/day (total 2400 mg/m² over 46-48 hours) continuous intravenous infusion. In some embodiments, the above therapeutically effective regimen of FOLFOX is repeated every several days, for example, every 7 days, 14 days, or 21 days. In some embodiments, a therapeutically effective regimen of FOLFOX comprises: Day 1 oxaliplatin of about 85 mg/m² IV infusion and leucovorin of about 200 mg/m² IV infusion both given over 120 minutes at the same time in separate bags, followed by 5-FU of about 400 mg/m² IV bolus given over 2-4 minutes, followed by 5-FU of about 600 mg/m² IV infusion in 500 mL D5W as a 22-hour continuous infusion; Day 2 leucovorin of about 200 mg/m² IV infusion over 120 minutes, followed by 5-FU of about 400 mg/m² IV bolus given over 2-4 minutes, followed by 5-FU of about 600 mg/m² IV infusion as a 22-hour continuous infusion. In some embodiments, a therapeutically effective regimen of FOLFOX comprises: Day 1-2 oxaliplatin of about 100 mg/m² given as a 120 minute IV infusion, concurrent with leucovorin of about 400 mg/m² (or levoleucovorin of about 200 mg/m²) IV infusion, followed by 5-FU of about 400 mg/m² IV bolus, followed by 46-hour 5-FU infusion (about 2400 mg/m² for first two cycles, increased to about 3000 mg/m² in case of no toxicity); Days 3-14: rest. In some embodiments FOLFOX is administered bi-weekly.

In some embodiments, according to any of the methods described herein, a therapeutically effective FOLFOX regimen comprises oxaliplatin, leucovorin, and 5-fluorouracil (5-FU), wherein oxaliplatin, leucovorin and 5-fluorouracil are administered with a specific dosing and administration schedule. In some embodiments, a therapeutically effective FOLFOX regimen is selected from a group consisting of a FOLFOX4 regimen, a FOLFOX6 regimen, a FOLFOX7 regimen, a modified FOLFOX4 regimen (mFOLFOX4), a modified FOLFOX6 regimen (mFOLFOX6), and a modified FOLFOX7 regimen (mFOLFOX7). See Table 1 for exemplary FOLFOX regimens. Various FOLFOX regimens or modifications to FOLFOX regimens not limited to those listed in Table 1 are known, or can be obtained by skilled in the art without undue experimentations. For example, see Kim et al., Oncol Lett. 2012 February; 3(2): 425-428; Mitchell et al., Clin Colorectal Cancer. 2006 July; 6(2):146-51.

TABLE 1

| Regimen | Dosing |
|---|---|
| FOLFOX4 | Day 1: Oxaliplatin 85 mg/m² IV and Leucovorin 200 mg/m² IV over 2 hours, |
| | followed by 5-FU 400 mg/m² bolus IV over 2-4 minutes, and followed by 5- |
| | FU 600 mg/m² IV over 22-hour continuous infusion. |
| | Day 2: Leucovorin 200 mg/m² IV over 2 hours, followed by 5-FU 400 mg/m² |
| | bolus IV over 2-4 minutes, and followed by 5-FU 600 mg/m² IV over 22- |
| | hour continuous infusion. |
| | Days 3-14: Rest days. |
| FOLFOX6 | Day 1-2: Oxaliplatin 100 mg/m² IV and Leucovorin 400 mg/m² IV over 2 |
| | hours, followed by 5-FU 400 mg/m² bolus IV, and followed by 5-FU 2400- |
| | 3000 mg/m² IV over 46-hour continuous infusion. |
| | Days 3-14: Rest days. |
| FOLFOX7 | Day 1-2: Oxaliplatin 130 mg/m² IV and Leucovorin 400 mg/m² IV over 2 |
| | hours, followed by 5-FU 2400 mg/m² IV over 46-hour continuous infusion. |
| | Days 3-14: Rest days. |
| Exemplary | Day 1: Oxaliplatin 85 mg/m² IV over 2 hours and Leucovorin 400 mg/m² IV |
| mFOLFOX6 | over 2 hours |
| | Days 1-3: 5-FU 400 mg/m² IV bolus on day 1, then 1,200 mg/m²/day × 2 days |
| | (total 2,400 mg/m² over 46-48 hours) IV continuous infusion. |
| | Day 4-14: Rest days. |

### Dosing and Method of Administering

The dose of the mTOR inhibitor nanoparticle composition (such as sirolimus/albumin nanoparticle composition) administered to an individual (e.g., a human) in combination therapy may vary with the particular composition, the method of administration, and the particular stage of colon cancer being treated. The amount should be sufficient to produce a desirable response, such as a therapeutic or prophylactic response against colon cancer. In some embodiments, the amount of mTOR inhibitor (such as a limus drug, e.g., sirolimus or a derivative thereof) in the composition is below the level that induces a toxicological effect (e.g., an effect above a clinically acceptable level of toxicity) or is at a level where a potential side effect can be controlled or tolerated when the mTOR inhibitor nanoparticle composition is administered to the individual.

In some embodiments, the mTOR inhibitor and the anti-VEGF antibody and/or at least a portion of the FOLFOX regimen are administered simultaneously to the individual. In some embodiments, the anti-VEGF antibody and at least a portion of the FOLFOX regimen are administered simultaneously to the individual. For example, the mTOR inhibitor nanoparticle compositions and the anti-VEGF antibody and/or at least a portion of the FOLFOX regimen are administered with a time separation of no more than about 15 minutes, such as no more than about any of 10, 5, or 1 minutes. In one example, wherein the compounds are in solution, simultaneous administration can be achieved by administering a solution containing the combination of compounds. In another example, simultaneous administration of separate solutions, one of which contains the mTOR inhibitor nanoparticle composition (such as sirolimus/albumin nanoparticle composition) and the other of which contains the anti-VEGF antibody and/or at least a portion of the FOLFOX regimen, can be employed. In one example, simultaneous administration can be achieved by administering a composition containing the combination of compounds. In another example, simultaneous administration can be achieved by administering two separate compositions, one comprising the mTOR inhibitor nanoparticle composition (such as sirolimus/albumin nanoparticle composition) and the other comprising the anti-VEGF antibody and/or at least a portion of the FOLFOX regimen. In some embodiments, simultaneous administration of the mTOR inhibitor (such as a limus drug, e.g., sirolimus or a derivative thereof) in the nanoparticle composition and the anti-VEGF antibody and/or at least a portion of the FOLFOX regimen can be combined with supplemental doses of the mTOR inhibitor and/or the anti-VEGF antibody and/or at least a portion of the FOLFOX regimen.

In some embodiments, the mTOR inhibitor nanoparticle composition (such as sirolimus/albumin nanoparticle composition) and the anti-VEGF antibody and/or at least a portion of the FOLFOX regimen are not administered simultaneously. In some embodiments, the anti-VEGF antibody and at least a portion of the FOLFOX regimen are not administered simultaneously to the individual. In some embodiments, the mTOR inhibitor nanoparticle composition (such as sirolimus/albumin nanoparticle composition) is administered before the anti-VEGF antibody and/or at least a portion of the FOLFOX regimen. In some embodiments, the anti-VEGF antibody and/or at least a portion of the FOLFOX regimen is administered before the mTOR inhibitor nanoparticle composition (such as sirolimus/albumin nanoparticle composition). The time difference in non-simultaneous administrations can be greater than 1 minute, five minutes, 10 minutes, 15 minutes, 30 minutes, 45 minutes, 60 minutes, two hours, three hours, six hours, nine hours, 12 hours, 24 hours, 36 hours, or 48 hours. In some embodiments, the first administered compound is provided time to take effect on the patient before the second administered compound is administered. In some embodiments, the difference in time does not extend beyond the time for the first administered compound to complete its effect in the patient, or beyond the time the first administered compound is completely or substantially eliminated or deactivated in the patient.

In some embodiments, the administration of the mTOR inhibitor nanoparticle composition (such as sirolimus/albumin nanoparticle composition) and the anti-VEGF antibody and/or at least a portion of the FOLFOX regimen are concurrent, i.e., the administration period of the mTOR inhibitor nanoparticle composition and that of the anti-VEGF antibody and/or at least a portion of the FOLFOX regimen overlap with each other. In some embodiments, the administration of the anti-VEGF antibody and at least a portion of the FOLFOX regimen are concurrent. In some embodiments, the mTOR inhibitor nanoparticle composition (such as sirolimus/albumin nanoparticle composition) is administered for at least one cycle (for example, at least any of 2, 3, or 4 cycles) prior to the administration of the anti-VEGF antibody and/or at least a portion of the FOLFOX regimen. In some embodiments, the anti-VEGF antibody and/or at least a portion of the FOLFOX regimen is administered for at least any of one, two, three, or four weeks. In some embodiments, the administrations of the mTOR inhibitor nanoparticle composition (such as sirolimus/albumin nanoparticle composition) and the anti-VEGF antibody and/or at least a portion of the FOLFOX regimen are initiated at about the same time (for example, within any one of 1, 2, 3, 4, 5, 6, or 7 days). In some embodiments, the administrations of the anti-VEGF antibody and at least a portion of the FOLFOX regimen are initiated at about the same time (for example, within any one of 1, 2, 3, 4, 5, 6, or 7 days). In some embodiments, the administrations of the mTOR inhibitor nanoparticle composition (such as sirolimus/albumin nanoparticle composition) and the anti-VEGF antibody and/or at least a portion of the FOLFOX regimen are terminated at about the same time (for example, within any one of 1, 2, 3, 4, 5, 6, or 7 days). In some embodiments, the administrations of the anti-VEGF antibody and/or at least a portion of the FOLFOX regimen are terminated at about the same time (for example, within any one of 1, 2, 3, 4, 5, 6, or 7 days). In some embodiments, the administration of the anti-VEGF antibody and/or at least a portion of the FOLFOX regimen continues (for example for about any one of 1, 2, 3, 4, 5, 6, 7, 8, 9, 10, 11, or 12 months) after the termination of the administration of the mTOR inhibitor nanoparticle composition (such as sirolimus/albumin nanoparticle composition). In some embodiments, the administration of the anti-VEGF antibody and/or at least a portion of the FOLFOX regimen is initiated after (for example after about any one of 1, 2, 3, 4, 5, 6, 7, 8, 9, 10, 11, or 12 months) the initiation of the administration of the mTOR inhibitor nanoparticle composition (such as sirolimus/albumin nanoparticle composition). In some embodiments, the administrations of the mTOR inhibitor nanoparticle composition (such as sirolimus/albumin nanoparticle composition) and the anti-VEGF antibody and/or at least a portion of the FOLFOX regimen are initiated and terminated at about the same time. In some embodiments, the administrations of the mTOR inhibitor nanoparticle composition (such as sirolimus/albumin nanoparticle composition) and the anti-VEGF antibody and/or at least a portion of the FOLFOX regimen are initiated at about the same time and the administration of the anti-VEGF antibody and/or at least a portion of the FOLFOX regimen continues (for example for about any one of 1, 2, 3, 4, 5, 6, 7, 8, 9, 10, 11, or 12 months) after the termination of the administration of the mTOR inhibitor nanoparticle composition. In some embodiments, the administration of the mTOR inhibitor nanoparticle composition (such as sirolimus/albumin nanoparticle composition) and the anti-VEGF antibody and/or at least a portion of the FOLFOX regimen stop at about the same time and the administration of the anti-VEGF antibody and/or at least a portion of the FOLFOX regimen is initiated after (for example after about any one of 1, 2, 3, 4, 5, 6, 7, 8, 9, 10, 11, or 12 months) the initiation of the administration of the mTOR inhibitor nanoparticle composition.

In some embodiments, the administration of the mTOR inhibitor nanoparticle composition (such as sirolimus/albumin nanoparticle composition) and the anti-VEGF antibody and/or at least a portion of the FOLFOX regimen are non-concurrent. In some embodiments, the administration of the anti-VEGF antibody and at least a portion of the FOLFOX regimen are non-concurrent. In some embodiments, the administration of the mTOR inhibitor nanoparticle composition (such as sirolimus/albumin nanoparticle composition) is terminated before the anti-VEGF antibody and/or at least a portion of the FOLFOX regimen is administered. In some embodiments, the administration of the anti-VEGF antibody and/or at least a portion of the FOLFOX regimen are terminated before the mTOR inhibitor nanoparticle composition (such as sirolimus/albumin nanoparticle composition) is administered. The time period between these two non-concurrent administrations can range from about two to eight weeks, such as about four weeks.

The dosing frequency of the mTOR inhibitor nanoparticle composition (such as sirolimus/albumin nanoparticle composition) and the anti-VEGF antibody and/or at least a portion of the FOLFOX regimen may be adjusted over the course of the treatment, based on the judgment of the administering physician. When administered separately, the mTOR inhibitor nanoparticle composition (such as sirolimus/albumin nanoparticle composition) and the anti-VEGF antibody and/or at least a portion of the FOLFOX regimen can be administered at different dosing frequency or intervals. For example, the mTOR inhibitor nanoparticle composition (such as sirolimus/albumin nanoparticle composition) can be administered weekly, while the anti-VEGF antibody and FOLFOX can be administered more or less frequently. In some embodiments, sustained continuous release formulation of the nanoparticle and/or the anti-VEGF antibody and/or FOLFOX may be used. Various formulations and devices for achieving sustained release are known in the art. A combination of the administration configurations described herein can also be used.

The mTOR inhibitor nanoparticle composition (such as sirolimus/albumin nanoparticle composition) and the anti-VEGF antibody and/or at least a portion of the FOLFOX regimen can be administered using the same route of administration or different routes of administration. In some embodiments (for both simultaneous and sequential administrations), the mTOR inhibitor (such as a limus drug, e.g., sirolimus or a derivative thereof) in the mTOR inhibitor nanoparticle composition and the anti-VEGF antibody and/or at least a portion of the FOLFOX regimen are administered at a predetermined ratio. For example, in some embodiments, the ratio by weight of the mTOR inhibitor (such as a limus drug, e.g., sirolimus or a derivative thereof) in the mTOR inhibitor nanoparticle composition and the anti-VEGF antibody or FOLFOX is about 1 to 1. In some embodiments, the weight ratio may be between about 0.001 to about 1 and about 1000 to about 1, or between about 0.01 to about 1 and 100 to about 1. In some embodiments, the ratio by weight of the mTOR inhibitor (such as a limus drug, e.g., sirolimus or a derivative thereof) in the mTOR inhibitor nanoparticle composition and the anti-VEGF antibody or FOLFOX is less than about any of 100:1, 50:1, 30:1, 10:1, 9:1, 8:1, 7:1, 6:1, 5:1, 4:1, 3:1, 2:1, and 1:1 In some embodiments, the ratio by weight of the mTOR inhibitor (such as a limus drug, e.g., sirolimus or a derivative thereof) in the mTOR inhibitor nanoparticle composition and the anti-VEGF antibody or FOLFOX is more than about any of 1:1, 2:1, 3:1, 4:1, 5:1, 6:1, 7:1, 8:1, 9:1, 30:1, 50:1, 100:1. Other ratios are contemplated.

The doses required for the mTOR inhibitor (such as a limus drug, e.g., sirolimus or a derivative thereof) in the mTOR inhibitor nanoparticle composition and/or the anti-VEGF antibody and/or at least a portion of the FOLFOX regimen may (but not necessarily) be the same or lower than what is normally required when each agent is administered alone. Thus, in some embodiments, a subtherapeutic amount of the mTOR inhibitor (such as a limus drug, e.g., sirolimus or a derivative thereof) in the mTOR inhibitor nanoparticle composition and/or the anti-VEGF antibody and/or at least a portion of the FOLFOX regimen is administered. “Subtherapeutic amount” or “subtherapeutic level” refer to an amount that is less than the therapeutic amount, that is, less than the amount normally used when the mTOR inhibitor nanoparticle composition (such as sirolimus/albumin nanoparticle composition) and/or the anti-VEGF antibody and/or at least a portion of the FOLFOX regimen are administered alone. The reduction may be reflected in terms of the amount administered at a given administration and/or the amount administered over a given period of time (reduced frequency).

In some embodiments, enough second therapeutic agent (such as anti-VEGF antibody and/or at least a component of FOLFOX) is administered so as to allow reduction of the normal dose of the mTOR inhibitor (such as a limus drug, e.g., sirolimus or a derivative thereof) in the mTOR inhibitor nanoparticle composition required to effect the same degree of treatment by at least about any of 5%, 10%, 20%, 30%, 50%, 60%, 70%, 80%, 90%, or more. In some embodiments, enough mTOR inhibitor (such as a limus drug, e.g., sirolimus or a derivative thereof) in the mTOR inhibitor nanoparticle composition is administered so as to allow reduction of the normal dose of the anti-VEGF antibody and/or the FOLFOX regimen required to effect the same degree of treatment by at least about any of 5%, 10%, 20%, 30%, 50%, 60%, 70%, 80%, 90%, or more.

In some embodiments, the dose of both the mTOR inhibitor (such as a limus drug, e.g., sirolimus or a derivative thereof) in the mTOR inhibitor nanoparticle composition and the anti-VEGF antibody and/or the FOLFOX regimen are reduced as compared to the corresponding normal dose of each when administered alone. In some embodiments, the mTOR inhibitor (such as a limus drug, e.g., sirolimus or a derivative thereof) in the mTOR inhibitor nanoparticle composition and/or the anti-VEGF antibody and/or the FOLFOX regimen are administered at a subtherapeutic, i.e., reduced level. In some embodiments, the dose of the mTOR inhibitor (such as a limus drug, e.g., sirolimus or a derivative thereof) in the mTOR inhibitor nanoparticle composition and/or the anti-VEGF antibody and/or the FOLFOX regimen is substantially less than the established maximum toxic dose (MTD). For example, the dose of the mTOR inhibitor nanoparticle composition (such as sirolimus/albumin nanoparticle composition) and/or the anti-VEGF antibody and/or at least the FOLFOX regimen is less than about 50%, 40%, 30%, 20%, or 10% of the MTD.

A combination of the administration configurations described herein can be used. The combination therapy methods described herein may be performed alone or in conjunction with another therapy, such as surgery, radiation, gene therapy, immunotherapy, bone marrow transplantation, stem cell transplantation, hormone therapy, targeted therapy, cryotherapy, ultrasound therapy, photodynamic therapy, and/or chemotherapy and the like.

As will be understood by those of ordinary skill in the art, the appropriate doses of the anti-VEGF antibody and the FOLFOX regimen will be approximately those already employed in clinical therapies wherein the anti-VEGF antibody or at least a portion of the FOLFOX regimen is administered alone or in combination. Variation in dosage will likely occur depending on the condition being treated. As described above, in some embodiments, the anti-VEGF antibody and the FOLFOX regimen may be administered at a reduced level.

In some embodiments, according to any of the methods described herein, the amount of the anti-VEGF antibody is about 1 mg/kg to 5 mg/kg, 1 mg/kg to 10 mg/kg, 1 mg/kg to 15 mg/kg, 1 mg/kg to 20 mg/kg, 1 mg/kg to 25 mg/kg, 1 mg/kg to 30 mg/kg, 5 mg/kg to 10 mg/kg, 5 mg/kg to 15 mg/kg, 5 mg/kg to 20 mg/kg, 5 mg/kg to 25 mg/kg, 5 mg/kg to 30 mg/kg, 10 mg/kg to 15 mg/kg, 10 mg/kg to 20 mg/kg, 10 mg/kg to 25 mg/kg, 10 mg/kg to 30 mg/kg, 15 mg/kg to 20 mg/kg, 15 mg/kg to 25 mg/kg, 15 mg/kg to 30 mg/kg, 20 mg/kg to 25 mg/kg, 20 mg/kg to 30 mg/kg or 25 mg/kg to 30 mg/kg. In some embodiments, the amount of the anti-VEGF antibody is about 5 mg/kg or 10 mg/kg. In some embodiments, the anti-VEGF antibody is administered intravenously, intraarterially, intraperitoneally, intravesicularly, subcutaneously, intrathecally, intrapulmonarily, intramuscularly, intratracheally, intraocularly, transdermally, orally, or by inhalation. In some embodiments, the anti-VEGF antibody is administered intravenously. In some embodiments, the anti-VEGF antibody is administered once weekly, every two weeks, once every three weeks, or once every four weeks. In some embodiments, the anti-VEGF antibody is administered once monthly, once every two months, once every three months, or once more than every three months. In some embodiments, the anti-VEGF antibody is administered as a dose of about 1 mg/kg to about 20 mg/kg (including for example about 5 mg/kg to 15 mg/kg, or about 10 mg/kg) at least 1, 2, 3, 4, 5, 6, 7, 8, 9, or more cycles, wherein every cycle consists of at least 2 weeks (such as at least any of 3, 4 weeks, or 1, 2, 3, 4, 5, 6 months). In some embodiments, the anti-VEGF antibody is administered as a dose of no more than about 20 mg/kg (such as no more than about any of 17.5, 15, 12.5, 10, 7.5, 5, 2.5 or less) mg in a cycle for at least one (such as at least any of 2, 3, 4, 5, 6, 7, 8, 9, 10 or more) cycle. In some embodiments, about 10 mg/kg of the anti-VEGF antibody is administered intravenously once every two weeks. In some embodiments, about 10 mg/kg of the anti-VEGF antibody is administered intravenously once every two weeks. In some embodiments, about 5 mg/kg of the anti-VEGF antibody is administered intravenously once every two weeks. The dose of the anti-VEGF antibody may be discontinued or interrupted, with or without dose reduction, to manage adverse drug reactions. In some embodiments, the anti-VEGF antibody is administered according to the prescribing information of an approved brand of the anti-VEGF antibody.

Whether administered in therapeutic or sub-therapeutic amounts, the combination of the mTOR inhibitor nanoparticle composition (such as sirolimus/albumin nanoparticle composition) and the anti-VEGF antibody and/or the FOLFOX regimen should be effective in treating a colon cancer. For example, a sub-therapeutic amount of an mTOR inhibitor nanoparticle composition (such as sirolimus/albumin nanoparticle composition) can be an effective amount if, when combined with a second therapeutic agent (such as anti-VEGF antibody and/or at least a component of FOLFOX), the combination is effective in the treatment of the colon cancer, and vice versa.

The dose of the mTOR inhibitor nanoparticle composition (such as sirolimus/albumin nanoparticle composition) and the dose of the anti-VEGF antibody and/or the FOLFOX regimen administered to an individual (such as a human) may vary with the particular composition, the mode of administration, and the type of colon cancer being treated. In some embodiments, the doses are effective to result in an objective response (such as a partial response or a complete response). In some embodiments, the doses are sufficient to result in a complete response in the individual. In some embodiments, the doses are sufficient to result in a partial response in the individual. In some embodiments, the doses administered are sufficient to produce an overall response rate of more than about any of 20%, 25%, 30%, 35%, 40%, 45%, 50%, 55%, 60%, 64%, 65%, 70%, 75%, 80%, 85%, or 90% among a population of individuals treated with the mTOR inhibitor nanoparticle composition (such as sirolimus/albumin nanoparticle composition) and the anti-VEGF antibody and/or the FOLFOX regimen. Responses of an individual to the treatment of the methods described herein can be determined, for example, based on RECIST levels.

In some embodiments, the amounts of the mTOR inhibitor nanoparticle composition (such as sirolimus/albumin nanoparticle composition) and the anti-VEGF antibody and/or the FOLFOX regimen are sufficient to prolong progress-free survival of the individual. In some embodiments, the amounts of the mTOR inhibitor nanoparticle composition (such as sirolimus/albumin nanoparticle composition) and the anti-VEGF antibody and/or the FOLFOX regimen are sufficient to prolong overall survival of the individual. In some embodiments, the amounts of the mTOR inhibitor nanoparticle composition (such as sirolimus/albumin nanoparticle composition) and the anti-VEGF antibody and/or the FOLFOX regimen are sufficient to produce clinical benefit of more than about any of 50%, 60%, 70%, or 77% among a population of individuals treated with the mTOR inhibitor nanoparticle composition and the anti-VEGF antibody and/or the FOLFOX regimen.

In some embodiments, the amounts of the mTOR inhibitor nanoparticle composition (such as sirolimus/albumin nanoparticle composition) and the anti-VEGF antibody and/or the FOLFOX regimen are sufficient to decrease the size of a tumor, decrease the number of cancer cells, or decrease the growth rate of a tumor by at least about any of 10%, 20%, 30%, 40%, 50%, 60%, 70%, 80%, 90%, 95% or 100% compared to the corresponding tumor size, number of cancer cells, or tumor growth rate in the same individual prior to treatment or compared to the corresponding activity in other individuals not receiving the treatment. Standard methods can be used to measure the magnitude of this effect, such as in vitro assays with purified enzyme, cell-based assays, animal models, or human testing.

In some embodiments, the amounts of the mTOR inhibitor nanoparticle composition (such as sirolimus/albumin nanoparticle composition) and the anti-VEGF antibody and/or the FOLFOX regimen are below the levels that induce a toxicological effect (i.e., an effect above a clinically acceptable level of toxicity) or are at a level where a potential side effect can be controlled or tolerated when the mTOR inhibitor nanoparticle composition and the anti-VEGF antibody and/or the FOLFOX regimen are administered to the individual.

In some embodiments, the amount of the mTOR inhibitor nanoparticle composition (such as sirolimus/albumin nanoparticle composition) is close to a maximum tolerated dose (MTD) of the composition following the same dosing regimen when administered with the anti-VEGF antibody and/or at least a portion of the FOLFOX regimen. In some embodiments, the amount of the mTOR inhibitor nanoparticle composition (such as sirolimus/albumin nanoparticle composition) is more than about any of 50%, 60%, 70%, 80%, 90%, 95%, or 98% of the MTD when administered with the anti-VEGF antibody and/or the FOLFOX regimen.

In some embodiments, the amount of an mTOR inhibitor (such as a limus drug, e.g., sirolimus) in the mTOR inhibitor nanoparticle composition is included in any of the following ranges: about 0.1 mg to about 1000 mg, about 0.1 mg to about 2.5 mg, about 0.5 mg to about 5 mg, about 5 mg to about 10 mg, about 10 mg to about 15 mg, about 15 mg to about 20 mg, about 20 mg to about 25 mg, about 20 mg to about 50 mg, about 25 mg to about 50 mg, about 50 mg to about 75 mg, about 50 mg to about 100 mg, about 75 mg to about 100 mg, about 100 mg to about 125 mg, about 125 mg to about 150 mg, about 150 mg to about 175 mg, about 175 mg to about 200 mg, about 200 mg to about 225 mg, about 225 mg to about 250 mg, about 250 mg to about 300 mg, about 300 mg to about 350 mg, about 350 mg to about 400 mg, about 400 mg to about 450 mg, or about 450 mg to about 500 mg, about 500 mg to about 600 mg, about 600 mg to about 700 mg, about 700 mg to about 800 mg, about 800 mg to about 900 mg, or about 900 mg to about 1000 mg, including any range between these values. In some embodiments, the amount of an mTOR inhibitor (such as a limus drug, e.g., sirolimus) in the effective amount of the composition (e.g., a unit dosage form) is in the range of about 5 mg to about 500 mg, such as about 30 to about 400 mg, 30 mg to about 300 mg, or about 50 mg to about 200 mg. In some embodiments, the amount of an mTOR inhibitor (such as a limus drug, e.g., sirolimus) in the effective amount of the mTOR inhibitor nanoparticle composition (e.g., a unit dosage form) is in the range of about 150 mg to about 500 mg, including for example, about 150 mg, about 225 mg, about 250 mg, about 300 mg, about 325 mg, about 350 mg, about 375 mg, about 400 mg, about 425 mg, about 450 mg, about 475 mg, or about 500 mg. In some embodiments, the concentration of the mTOR inhibitor (such as a limus drug, e.g., sirolimus) in the mTOR inhibitor nanoparticle composition is dilute (about 0.1 mg/ml) or concentrated (about 100 mg/ml), including for example about any of 0.1 mg/ml to about 50 mg/ml, about 0.1 mg/ml to about 20 mg/ml, about 1 mg/ml to about 10 mg/ml, about 2 mg/ml to about 8 mg/ml, about 4 mg/ml to about 6 mg/ml, or about 5 mg/ml. In some embodiments, the concentration of the mTOR inhibitor (such as a limus drug, e.g., sirolimus) in the mTOR inhibitor nanoparticle composition is at least about any of 0.5 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, or 50 mg/ml.

In some embodiments of any of the above aspects, the amount of an mTOR inhibitor (such as a limus drug, e.g., sirolimus) in the mTOR inhibitor nanoparticle composition is at least about any of 1 mg/kg, 2.5 mg/kg, 3.5 mg/kg, 5 mg/kg, 6.5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, or 60 mg/kg. In some embodiments, the effective amount of an mTOR inhibitor (such as a limus drug, e.g., sirolimus) in the mTOR inhibitor nanoparticle composition is less than about any of 350 mg/kg, 300 mg/kg, 250 mg/kg, 200 mg/kg, 150 mg/kg, 100 mg/kg, 50 mg/kg, 25 mg/kg, 20 mg/kg, 10 mg/kg, 7.5 mg/kg, 6.5 mg/kg, 5 mg/kg, 3.5 mg/kg, 2.5 mg/kg, or 1 mg/kg.

In some embodiments of any of the above aspects, the amount of an mTOR inhibitor (such as a limus drug, e.g., sirolimus) in the mTOR inhibitor nanoparticle composition is about any of 10 mg/m², 15 mg/m², 20 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 90 mg/m², 100 mg/m², 120 mg/m², 160 mg/m², 175 mg/m², 180 mg/m², 200 mg/m², 210 mg/m², 220 mg/m², 250 mg/m², 260 mg/m², 300 mg/m², 350 mg/m², 400 mg/m², 500 mg/m², 540 mg/m², 750 mg/m², 1000 mg/m², or 1080 mg/m² mTOR inhibitor. In some embodiments, the mTOR inhibitor nanoparticle composition includes less than about any of 350 mg/m², 300 mg/m², 250 mg/m², 200 mg/m², 150 mg/m², 120 mg/m², 100 mg/m², 90 mg/m², 50 mg/m², or 30 mg/m² mTOR inhibitor (such as a limus drug, e.g., sirolimus). In some embodiments, the amount of the mTOR inhibitor (such as a limus drug, e.g., sirolimus) per administration is less than about any of 25 mg/m², 22 mg/m², 20 mg/m², 18 mg/m², 15 mg/m², 14 mg/m², 13 mg/m², 12 mg/m², 11 mg/m², 10 mg/m², 9 mg/m², 8 mg/m², 7 mg/m², 6 mg/m², 5 mg/m², 4 mg/m², 3 mg/m², 2 mg/m², or 1 mg/m². In some embodiments, the effective amount of mTOR inhibitor (such as a limus drug, e.g., sirolimus) in the mTOR inhibitor nanoparticle composition is included in any of the following ranges: about 1 to about 5 mg/m², about 5 to about 10 mg/m², about 10 to about 30 mg/m², about 30 to about 45 mg/m², about 45 to about 75 mg/m², about 75 to about 100 mg/m², about 100 to about 125 mg/m², about 125 to about 150 mg/m², about 150 to about 175 mg/m², about 175 to about 200 mg/m², about 200 to about 225 mg/m², about 225 to about 250 mg/m², about 250 to about 300 mg/m², about 300 to about 350 mg/m², or about 350 to about 400 mg/m². In some embodiments, the effective amount of mTOR inhibitor (such as a limus drug, e.g., sirolimus) in the mTOR inhibitor nanoparticle composition is about 30 to about 300 mg/m², such as about 100 to about 150 mg/m², about 120 mg/m², about 130 mg/m², or about 140 mg/m².

In some embodiments, the effective amount of mTOR inhibitor (such as a limus drug, e.g., sirolimus) in the mTOR inhibitor nanoparticle composition is in any of the following ranges: about 10 to about 20 mg/m², about 10 to about 30 mg/m², about 10 to about 45 mg/m², about 10 to about 60 mg/m², about 20 to about 30 mg/m², about 20 to about 45 mg/m², about 20 to about 60 mg/m², about 30 to about 45 mg/m², about 30 to about 60 mg/m², or about 45 to about 60 mg/m², each inclusive. In some embodiments, the dosing frequency for the administration of the mTOR inhibitor nanoparticle composition (such as sirolimus/albumin nanoparticle composition) is three out of four weeks.

In some embodiments, the FOLFOX regimen is administered for at least one (such as at least any of 2, 3, 4, 5, 6, 7, 8, 9, 10, 11, 12 or more) cycle. In some embodiments, the FOLFOX regimen is administered for at most 12 (such as at most any of 11, 10, 9, 8, 7, 6 or less) cycles. The FOLFOX regimen may be discontinued or interrupted, with or without dose reduction, to manage adverse drug reactions.

In some embodiments, the FOLFOX regimen is FOLFOX4, the anti-VEGF antibody is administered intravenously, once every two weeks with an amount of about 10 mg/kg, and the the amount of the mTOR inhibitor in the mTOR inhibitor nanoparticle composition is from about 10 mg/m² to about 100 mg/m². In some embodiments, the FOLFOX regimen is FOLFOX4, the anti-VEGF antibody is administered intravenously, once every two weeks with an amount of about 10 mg/kg, and the amount of the mTOR inhibitor in the mTOR inhibitor nanoparticle composition is from about 10 mg/m² to about 30 mg/m². In some embodiments, the FOLFOX regimen is FOLFOX4, the anti-VEGF antibody is administered intravenously, once every two weeks with an amount of about 10 mg/kg, and the amount of the mTOR inhibitor in the mTOR inhibitor nanoparticle composition is from about 30 mg/m² to about 45 mg/m². In some embodiments, the FOLFOX regimen is FOLFOX4, the anti-VEGF antibody is administered intravenously, once every two weeks with an amount of about 10 mg/kg, and the amount of the mTOR inhibitor in the mTOR inhibitor nanoparticle composition is from about 45 mg/m² to about 75 mg/m². In some embodiments, the FOLFOX regimen is FOLFOX4, the anti-VEGF antibody is administered intravenously, once every two weeks with an amount of about 10 mg/kg, and the amount of the mTOR inhibitor in the mTOR inhibitor nanoparticle composition is from about 75 mg/m² to about 100 mg/m².

In some embodiments, the FOLFOX regimen is a modified FOLFOX6 regimen, the anti-VEGF antibody is administered intravenously, once every two weeks with an amount of about 5 mg/kg, and the amount of the mTOR inhibitor in the mTOR inhibitor nanoparticle composition is from about 10 mg/m² to about 100 mg/m². In some embodiments, the FOLFOX regimen is a modified FOLFOX6 regimen, the anti-VEGF antibody is administered intravenously, once every two weeks with an amount of about 5 mg/kg, and the amount of the mTOR inhibitor in the mTOR inhibitor nanoparticle composition is from about 10 mg/m² to about 30 mg/m². In some embodiments, the FOLFOX regimen is a modified FOLFOX6 regimen, the anti-VEGF antibody is administered intravenously, once every two weeks with an amount of about 5 mg/kg, and the amount of the mTOR inhibitor in the mTOR inhibitor nanoparticle composition is from about 30 mg/m² to about 45 mg/m². In some embodiments, the FOLFOX regimen is a modified FOLFOX6 regimen, the anti-VEGF antibody is administered intravenously, once every two weeks with an amount of about 5 mg/kg, and the amount of the mTOR inhibitor in the mTOR inhibitor nanoparticle composition is from about 45 mg/m² to about 75 mg/m². In some embodiments, the FOLFOX regimen is a modified FOLFOX6 regimen, the anti-VEGF antibody is administered intravenously, once every two weeks with an amount of about 5 mg/kg, and the amount of the mTOR inhibitor in the mTOR inhibitor nanoparticle composition is from about 75 mg/m² to about 100 mg/m².

In some embodiments, the combination of compounds exhibits a synergistic effect (i.e., greater than additive effect) in the treatment of the colon cancer. The term “synergistic effect” refers to the action of two agents, such as an mTOR inhibitor nanoparticle composition (such as sirolimus/albumin nanoparticle composition) and a second therapeutic agent (such as anti-VEGF antibody and/or at least a component of FOLFOX), producing an effect, for example, slowing the symptomatic progression of cancer or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

In different embodiments, depending on the combination and the effective amounts used, the combination of compounds can inhibit cancer growth, achieve cancer stasis, or even achieve substantial or complete cancer regression.

While the amounts of an mTOR inhibitor nanoparticle composition (such as sirolimus/albumin nanoparticle composition) and an anti-VEGF antibody, and a FOLFOX regimen should result in the effective treatment of a colon cancer, the amounts, when combined, are preferably not excessively toxic to the individual (i.e., the amounts are preferably within toxicity limits as established by medical guidelines). In some embodiments, either to prevent excessive toxicity and/or provide a more efficacious treatment of a colon cancer, a limitation on the total administered dosage is provided.

Different dosage regimens may be used to treat a colon cancer. In some embodiments, a daily dosage, such as any of the exemplary dosages described above, is administered once, twice, three times, or four times a day for three, four, five, six, seven, eight, nine, ten, or more days. Depending on the stage and severity of the cancer, a shorter treatment time (e.g., up to five days) may be employed along with a high dosage, or a longer treatment time (e.g., ten or more days, or weeks, or a month, or longer) may be employed along with a low dosage. In some embodiments, a once- or twice-daily dosage is administered every other day.

In some embodiments, the dosing frequencies for the administration of the mTOR inhibitor nanoparticle composition (such as sirolimus/albumin nanoparticle composition) include, but are not limited to, daily, every two days, every three days, every four days, every five days, every six days, weekly without break, three out of four weeks (such as on days 1, 8, and 15 of a 28-day cycle), once every three weeks, once every two weeks, or two out of three weeks. In some embodiments, the mTOR inhibitor nanoparticle composition (such as sirolimus/albumin nanoparticle composition) is administered about once every 2 weeks, once every 3 weeks, once every 4 weeks, once every 6 weeks, or once every 8 weeks. In some embodiments, the mTOR inhibitor nanoparticle composition (such as sirolimus/albumin nanoparticle composition) is administered at least about any of 1×, 2×, 3×, 4×, 5×, 6×, or 7× (i.e., daily) a week. In some embodiments, the intervals between each administration are less than about any of 6 months, 3 months, 1 month, 20 days, 15, days, 14 days, 13 days, 12 days, 11 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

In some embodiments, the dosing frequency is once every two days for one time, two times, three times, four times, five times, six times, seven times, eight times, nine times, ten times, or eleven times. In some embodiments, the dosing frequency is once every two days for five times. In some embodiments, the mTOR inhibitor (such as a limus drug, e.g., sirolimus or a derivative thereof) is administered over a period of at least ten days, wherein the interval between each administration is no more than about two days, and wherein the dose of the mTOR inhibitor at each administration is about 0.25 mg/m² to about 250 mg/m², about 0.25 mg/m² to about 150 mg/m², about 0.25 mg/m² to about 75 mg/m², such as about 0.25 mg/m² to about 25 mg/m², or about 25 mg/m² to about 50 mg/m².

The administration of the mTOR inhibitor nanoparticle composition (such as sirolimus/albumin nanoparticle composition) can be extended over an extended period of time, such as from about a month up to about seven years. In some embodiments, the mTOR inhibitor nanoparticle composition (such as sirolimus/albumin nanoparticle composition) is administered over a period of at least about any of 2, 3, 4, 5, 6, 7, 8, 9, 10, 11, 12, 18, 24, 30, 36, 48, 60, 72, or 84 months.

In some embodiments, the dosage of an mTOR inhibitor (such as a limus drug, e.g., sirolimus or a derivative thereof) in a nanoparticle composition can be in the range of 5-400 mg/m² when given on a 3 week schedule, or 5-250 mg/m² (such as 80-150 mg/m², for example 100-120 mg/m²) when given on a weekly schedule. For example, the amount of an mTOR inhibitor (such as a limus drug, e.g., sirolimus or a derivative thereof) is about 60 to about 300 mg/m² (e.g., about 260 mg/m²) on a three week schedule.

In some embodiments, the exemplary dosing schedules for the administration of the mTOR inhibitor nanoparticle composition (such as sirolimus/albumin nanoparticle composition) include, but are not limited to, 100 mg/m², weekly, without break; 10 mg/m² weekly, 3 out of four weeks (such as on days 1, 8, and 15 of a 28-day cycle); 45 mg/m² weekly, 3 out of four weeks (such as on days 1, 8, and 15 of a 28-day cycle); 75 mg/m² weekly, 3 out of four weeks (such as on days 1, 8, and 15 of a 28-day cycle); 100 mg/m²,weekly, 3 out of 4 weeks; 125 mg/m², weekly, 3 out of 4 weeks; 125 mg/m², weekly, 2 out of 3 weeks; 130 mg/m², weekly, without break; 175 mg/m², once every 2 weeks; 260 mg/m², once every 2 weeks; 260 mg/m², once every 3 weeks; 180-300 mg/m², every three weeks; 60-175 mg/m², weekly, without break; 20-150 mg/m² twice a week; and 150-250 mg/m² twice a week. The dosing frequency of the mTOR inhibitor nanoparticle composition (such as sirolimus/albumin nanoparticle composition) may be adjusted over the course of the treatment based on the judgment of the administering physician.

In some embodiments, the individual is treated for at least about any of one, two, three, four, five, six, seven, eight, nine, or ten treatment cycles.

The mTOR inhibitor nanoparticle composition (such as sirolimus/albumin nanoparticle composition) described herein allow infusion of the mTOR inhibitor nanoparticle composition to an individual over an infusion time that is shorter than about 24 hours. For example, in some embodiments, the mTOR inhibitor nanoparticle composition (such as sirolimus/albumin nanoparticle composition) is administered over an infusion period of less than about any of 24 hours, 12 hours, 8 hours, 5 hours, 3 hours, 2 hours, 1 hour, 30 minutes, 20 minutes, or 10 minutes. In some embodiments, the mTOR inhibitor nanoparticle composition (such as sirolimus/albumin nanoparticle composition) is administered over an infusion period of about 30 minutes.

In some embodiments, the exemplary dose of the mTOR inhibitor (in some embodiments a limus drug, e.g., sirolimus) in the mTOR inhibitor nanoparticle composition includes, but is not limited to, about any of 50 mg/m², 60 mg/m², 75 mg/m², 80 mg/m², 90 mg/m², 100 mg/m², 120 mg/m², 160 mg/m², 175 mg/m², 200 mg/m², 210 mg/m², 220 mg/m², 260 mg/m², and 300 mg/m². For example, the dosage of an mTOR inhibitor (such as a limus drug, e.g., sirolimus or a derivative thereof) in a nanoparticle composition can be in the range of about 100-400 mg/m² when given on a 3 week schedule, or about 10-250 mg/m² when given on a weekly schedule.

In some embodiments, the dosage of an mTOR inhibitor (such as a limus drug, e.g., sirolimus) is about 100 mg to about 400 mg, for example about 100 mg, about 200 mg, about 300 mg, or about 400 mg. In some embodiments, the limus drug is administered at about 100 mg weekly, about 200 mg weekly, about 300 mg weekly, about 100 mg twice weekly, or about 200 mg twice weekly. In some embodiments, the administration is further followed by a monthly maintenance dose (which can be the same or different from the weekly doses).

In some embodiments when the limus nanoparticle composition is administered intravenously, the dosage of an mTOR inhibitor (such as a limus drug, e.g., sirolimus) in a nanoparticle composition can be in the range of about 30 mg to about 400 mg. The mTOR inhibitor nanoparticle composition (such as sirolimus/albumin nanoparticle composition) described herein allow infusion of the mTOR inhibitor nanoparticle composition to an individual over an infusion time that is shorter than about 24 hours. For example, in some embodiments, the mTOR inhibitor nanoparticle composition (such as sirolimus/albumin nanoparticle composition) is administered over an infusion period of less than about any of 24 hours, 12 hours, 8 hours, 5 hours, 3 hours, 2 hours, 1 hour, 30 minutes, 20 minutes, or 10 minutes. In some embodiments, the mTOR inhibitor nanoparticle composition (such as sirolimus/albumin nanoparticle composition) is administered over an infusion period of about 30 minutes to about 40 minutes.

In some embodiments, each dosage contains both an mTOR inhibitor nanoparticle composition (such as sirolimus/albumin nanoparticle composition) and an anti-VEGF antibody and/or at least a portion of the FOLFOX regimen to be delivered as a single dosage, while in other embodiments, each dosage contains either the mTOR inhibitor nanoparticle composition or the anti-VEGF antibody and/or at least a portion of the FOLFOX regimen to be delivered as separate dosages.

An mTOR inhibitor nanoparticle composition (such as sirolimus/albumin nanoparticle composition) and an anti-VEGF antibody and/or at least a portion of the FOLFOX regimen, in pure form or in an appropriate pharmaceutical composition, can be administered via any of the accepted modes of administration or agents known in the art. The compositions and/or agents can be administered, for example, orally, nasally, parenterally (such as intravenous, intramuscular, or subcutaneous), topically, transdermally, intravaginally, intravesically, intracistemally, or rectally. The dosage form can be, for example, a solid, semi-solid, lyophilized powder, or liquid dosage form, such as tablets, pills, soft elastic or hard gelatin capsules, powders, solutions, suspensions, suppositories, aerosols, or the like, preferably in unit dosage forms suitable for simple administration of precise dosages.

As discussed above, the mTOR inhibitor nanoparticle composition (such as sirolimus/albumin nanoparticle composition) and the anti-VEGF antibody and/or at least a portion of the FOLFOX regimen can be administered in a single unit dose or separate dosage forms. Accordingly, the phrase “pharmaceutical combination” includes a combination of two drugs in either a single dosage form or a separate dosage forms, i.e., the pharmaceutically acceptable carriers and excipients described throughout the application can be combined with an mTOR inhibitor nanoparticle composition (such as sirolimus/albumin nanoparticle composition) and a second therapeutic agent (such as anti-VEGF antibody and/or at least a component of FOLFOX) in a single unit dose, as well as individually combined with an mTOR inhibitor nanoparticle composition and a second therapeutic agent(such as anti-VEGF antibody and/or at least a component of FOLFOX) when these compounds are administered separately.

Auxiliary and adjuvant agents may include, for example, preserving, wetting, suspending, sweetening, flavoring, perfuming, emulsifying, and dispensing agents. Prevention of the action of microorganisms is generally provided by various antibacterial and antifungal agents, such as, parabens, chlorobutanol, phenol, sorbic acid, and the like. Isotonic agents, such as sugars, sodium chloride, and the like, may also be included. Prolonged absorption of an injectable pharmaceutical form can be brought about by the use of agents delaying absorption, for example, aluminum monostearate and gelatin. The auxiliary agents also can include wetting agents, emulsifying agents, pH buffering agents, and antioxidants, such as citric acid, sorbitan monolaurate, triethanolamine oleate, butylated hydroxytoluene, and the like.

Solid dosage forms can be prepared with coatings and shells, such as enteric coatings and others well-known in the art. They can contain pacifying agents and can be of such composition that they release the active compound or compounds in a certain part of the intestinal tract in a delayed manner. Examples of embedded compositions that can be used are polymeric substances and waxes. The active compounds also can be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., the mTOR inhibitor nanoparticle composition (such as sirolimus/albumin nanoparticle composition) or second therapeutic agents (such as anti-VEGF antibody and/or at least a component of FOLFOX) described herein, or a pharmaceutically acceptable salt thereof, and optional pharmaceutical adjuvants in a carrier, such as, for example, water, saline, aqueous dextrose, glycerol, ethanol and the like; solubilizing agents and emulsifiers, such as ethyl alcohol, isopropyl alcohol, ethyl carbonate, ethyl acetate, benzyl alcohol, benzyl benzoate, propyleneglycol, 1,3-butyleneglycol, dimethyl formamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

In some embodiments, depending on the intended mode of administration, the pharmaceutically acceptable compositions will contain about 1% to about 99% by weight of the compounds described herein, or a pharmaceutically acceptable salt thereof, and 99% to 1% by weight of a pharmaceutically acceptable excipient. In one example, the composition will be between about 5% and about 75% by weight of a compound described herein, or a pharmaceutically acceptable salt thereof, with the rest being suitable pharmaceutical excipients.

Actual methods of preparing such dosage forms are known, or will be apparent, to those skilled in this art. Reference is made, for example, to Remington's Pharmaceutical Sciences, 18^{th} Ed., (Mack Publishing Company, Easton, Pa., 1990).

In some embodiments, the mTOR inhibitor nanoparticle composition (such as sirolimus/albumin nanoparticle composition), the anti-VEGF antibody and the FOLFOX regimen can be administered with any of the following dosing regimen as in Table 2.

TABLE 2
Exemplary dosing regimen of combination therapy.

| | Exemplary dosing regimen |
|---|---|
| 1 | Sirolimus (i.e., rapamycin): about 10 mg/m² administered intravenously (IV) weekly |
| | for 3 weeks, followed by a week of rest. |
| | Bevacizumab: about 10 mg/kg IV, every two weeks. |
| | Modified FOLFOX6 regimen: Oxaliplatin of about 85 mg/m² IV with Leucovorin of |
| | about 400 mg/m² IV over 2 hours; 5-FU of about 400 mg/m² IV bolus followed by about |
| | 2,400 mg/m² IV continuous infusion over 46 hours every two weeks. |
| | (Dose modification of each agent in FOLFOX may be made independently based on |
| | the specific type of toxicities observed.) |
| 2 | Sirolimus (i.e., rapamycin): about 20 mg/m² administered intravenously (IV) weekly |
| | for 3 weeks, followed by a week of rest. |
| | Bevacizumab: about 10 mg/kg IV, every two weeks. |
| | Modified FOLFOX6 regimen: Oxaliplatin of about 85 mg/m² IV with Leucovorin of |
| | about 400 mg/m² IV over 2 hours; 5-FU of about 400 mg/m² IV bolus followed by about |
| | 2,400 mg/m² IV continuous infusion over 46 hours every two weeks. |
| | (Dose modification of each agent in FOLFOX may be made independently based on |
| | the specific type of toxicities observed.) |
| 3 | Sirolimus (i.e., rapamycin): about 30 mg/m² administered intravenously (IV) weekly |
| | for 3 weeks, followed by a week of rest. |
| | Bevacizumab: about 10 mg/kg IV, every two weeks. |
| | Modified FOLFOX6 regimen: Oxaliplatin of about 85 mg/m² IV with Leucovorin of |
| | about 400 mg/m² IV over 2 hours; 5-FU of about 400 mg/m² IV bolus followed by about |
| | 2,400 mg/m² IV continuous infusion over 46 hours every two weeks. |
| | (Dose modification of each agent in FOLFOX may be made independently based on |
| | the specific type of toxicities observed.) |
| 4 | Sirolimus (i.e., rapamycin): about 45 mg/m² administered intravenously (IV) weekly |
| | for 3 weeks, followed by a week of rest. |
| | Bevacizumab: about 10 mg/kg IV, every two weeks. |
| | Modified FOLFOX6 regimen: Oxaliplatin of about 85 mg/m² IV with Leucovorin of |
| | about 400 mg/m² IV over 2 hours; 5-FU of about 400 mg/m² IV bolus followed by about |
| | 2,400 mg/m² IV continuous infusion over 46 hours every two weeks. |
| | (Dose modification of each agent in FOLFOX may be made independently based on |
| | the specific type of toxicities observed.) |
| 5 | Sirolimus (i.e., rapamycin): about 60 mg/m² administered intravenously (IV) weekly |
| | for 3 weeks, followed by a week of rest. |
| | Bevacizumab: about 10 mg/kg IV, every two weeks. |
| | Modified FOLFOX6 regimen: Oxaliplatin of about 85 mg/m² IV with Leucovorin of |
| | about 400 mg/m² IV over 2 hours; 5-FU of about 400 mg/m² IV bolus followed by about |
| | 2,400 mg/m² IV continuous infusion over 46 hours every two weeks. |
| | (Dose modification of each agent in FOLFOX may be made independently based on |
| | the specific type of toxicities observed.) |
| 6 | Sirolimus (i.e., rapamycin): about 10 to about 60 mg/m² IV |
| | Bevacizumab: about 5 mg/kg to about 10 mg/kg IV |
| | Modified FOLFOX6 regimen: Oxaliplatin of about 85 mg/m² IV with Leucovorin of |
| | about 400 mg/m² IV over 2 hours; 5-FU of about 400 mg/m² IV bolus followed by about |
| | 2,400 mg/m² IV continuous infusion over 46 hours in two weeks. |
| | (Dose modification of each agent in FOLFOX may be made independently based on |
| | the specific type of toxicities observed.) |
| 7 | Sirolimus (i.e., rapamycin): about 30 mg/m² administered intravenously (IV) once |
| | every two weeks; |
| | Bevacizumab: about 5 mg/kg to about 10 mg/kg IV (such as about 5 mg/kg IV) |
| | Modified FOLFOX6 regimen: Oxaliplatin of about 85 mg/m² IV with Leucovorin of |
| | about 400 mg/m² IV over 2 hours; 5-FU of about 400 mg/m² IV bolus followed by about |
| | 2,400 mg/m² IV continuous infusion over 46 hours in two weeks. |
| | (Dose modification of each agent in FOLFOX may be made independently based on |
| | the specific type of toxicities observed.) |

Treatments according to any dosing regimen such as the exemplary dosing regimens discussed above can be repeated for multiple cycles (such as 1, 2, 3, 4, 5, 6, or more cycles, such as about 1-10 cycles, 1-7 cycles, 1-5 cycles, 1-4 cycles, 1-3 cycles). In some embodiments, the treatment according to a specific dosing regiment is repeated for at least two, three or more cycles. In some embodiments, the treatment according to a specific dosing regimen is continuously repeated (i.e., without an interval) for at least two, three or more cycles.

In some embodiments, there is an interval between two adjacent cycles. In some embodiments, the interval is at least about one, two, three or four weeks. In some embodiments, the interval is at least about one, two, three, four, five, six or more months. In some embodiments, the interval is about a time period that allows the individual to gain weight (for example, the individual has a weight of about or at least about 90%, 92%, 95%, 97% of the weight prior to the initiation of the treatment(s) after the interval).

The mTOR inhibitor nanoparticle composition (such as sirolimus/albumin nanoparticle composition) can be administered to an individual (such as a human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, sustained continuous release formulation of the composition may be used. In some embodiments, the composition is administered intravenously. In some embodiments, the composition is administered intraportally. In some embodiments, the composition is administered intraarterially. In some embodiments, the composition is administered intraperitoneally.

### Patient Population

In some embodiments, the individual is at least about 50, 55, or 60 years old.

In some embodiments, the individual has a history of smoking. In some embodiments, the individual has a history of smoking for at least about 5, 10, 15, 20, 25, 30, 35, or 40 years.

In some embodiments, the individual has a metastatic colorectal cancer. In some embodiments, the cancer has metastasized to one, two, three or more other organs (e.g., pancreas, lung, liver, kidney, brain).

### Articles of Manufacture and Kits

In some embodiments of the invention, there is provided an article of manufacture containing materials useful for the treatment of a colon cancer comprising an mTOR inhibitor nanoparticle composition (such as sirolimus/albumin nanoparticle composition) and an anti-VEGF antibody and a FOLFOX regimen. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). At least one active agent in the composition is a) a nanoparticle formulation of an mTOR inhibitor; b) an anti-VEGF antibody; or c) at least a portion of FOLFOX regimen. The label or package insert indicates that the composition is used for treating the particular condition in an individual. The label or package insert will further comprise instructions for administering the composition to the individual. Articles of manufacture and kits comprising combination therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating a colon cancer.

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a colon cancer. Kits of the invention include one or more containers comprising an mTOR inhibitor nanoparticle composition (such as sirolimus/albumin nanoparticle composition) (or unit dosage form and/or article of manufacture), and in some embodiments, further comprise an anti-VEGF antibody and/or at least a portion of a FOLFOX regimen and/or instructions for use in accordance with any of the methods described herein. The kit may further comprise a description of selection of individuals suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

For example, in some embodiments, the kit comprises a composition comprising an mTOR inhibitor nanoparticle composition (such as sirolimus/albumin nanoparticle composition). In some embodiments, the kit comprises a) a composition comprising an mTOR inhibitor nanoparticle composition (such as sirolimus/albumin nanoparticle composition), and b) an anti-VEGF antibody (e.g., bevacizumab) and/or at least a portion of FOLFOX regimen. In some embodiments, the kit comprises a) a composition comprising an mTOR inhibitor nanoparticle composition (such as sirolimus/albumin nanoparticle composition), and b) instructions for administering the mTOR inhibitor nanoparticle composition in combination with an anti-VEGF antibody (e.g., bevacizumab) and a FOLFOX regimen to an individual for treatment of a colon cancer. In some embodiments, the kit comprises a) a composition comprising an mTOR inhibitor nanoparticle composition (such as sirolimus/albumin nanoparticle composition), b) an anti-VEGF antibody, and c) instructions for administering the mTOR inhibitor nanoparticle composition and an anti-VEGF antibody (e.g., bevacizumab) and/or a FOLFOX regimen to an individual for treatment of a colon cancer. In some embodiments, the kit comprises a) a composition comprising an mTOR inhibitor nanoparticle composition (such as sirolimus/albumin nanoparticle composition), b) an anti-VEGF antibody, c) at least a portion of FOLFOX regimen, and d) instructions for administering the mTOR inhibitor nanoparticle composition and an anti-VEGF antibody (e.g., bevacizumab) and/or a FOLFOX regimen to an individual for treatment of a colon cancer. The mTOR inhibitor nanoparticle composition (such as sirolimus/albumin nanoparticle composition) and an anti-VEGF antibody (e.g., bevacizumab) and/or at least a portion of FOLFOX regimen can be present in separate containers or in a single container. For example, the kit may comprise one distinct composition or two or more compositions wherein one composition comprises an mTOR inhibitor nanoparticle composition (such as sirolimus/albumin nanoparticle composition) and another composition comprises the anti-VEGF antibody (e.g., bevacizumab) and/or at least a portion of FOLFOX regimen.

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the mTOR inhibitor nanoparticle composition (such as sirolimus/albumin nanoparticle composition) and the anti-VEGF antibody (e.g., bevacizumab) and/or at least a portion of FOLFOX regimen generally include information as to dosage, dosing schedule, and route of administration for the intended treatment. The containers may be unit doses, bulk packages (e.g., multi-dose packages) or sub-unit doses. For example, kits may be provided that contain sufficient dosages of an mTOR inhibitor nanoparticle composition (such as sirolimus/albumin nanoparticle composition) and an anti-VEGF antibody (e.g., bevacizumab) and/or at least a portion of FOLFOX regimen as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the mTOR inhibitor nanoparticle composition (such as sirolimus/albumin nanoparticle composition) and anti-VEGF antibody (e.g., bevacizumab) and/or the FOLFOX regimen and instructions for use, packaged in quantities sufficient for storage and use in pharmacies, for example, hospital pharmacies and compounding pharmacies.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### Exemplary Embodiments

Embodiment 1. A method of treating a colon cancer in an individual, comprising administering to the individual: a) an effective amount of a composition comprising nanoparticles comprising an mTOR inhibitor and an albumin, b) an effective amount of anti-VEGF antibody, c) a therapeutically effective FOLFOX regimen.

Embodiment 2. The method of embodiment 1, wherein the colon cancer comprises an mTOR-activation aberration.

Embodiment 3. The method of embodiment 2, wherein the mTOR-activation aberration comprises a PTEN aberration.

Embodiment 4. The method of embodiment 3, wherein the mTOR-activation aberration further comprises a KRAS aberration.

Embodiment 5. The method of embodiment 3, wherein the mTOR-activation aberration further comprises a second aberration, wherein the second aberration is not a PTEN or a KRAS aberration.

Embodiment 6. The method of embodiment 1-5, wherein the mTOR inhibitor is a limus drug.

Embodiment 7. The method of embodiment 6, wherein the limus drug is rapamycin.

Embodiment 8. The method of any one of embodiments 1-7, wherein the anti-VEGF antibody is bevacizumab.

Embodiment 9. The method of any one of embodiments 1-8, wherein the amount of the mTOR inhibitor in the mTOR inhibitor nanoparticle composition is from about 10 mg/m² to about 30 mg/m².

Embodiment 10. The method of any one of embodiments 1-8, wherein the amount of the mTOR inhibitor in the mTOR inhibitor nanoparticle composition is from about 30 mg/m² to about 45 mg/m².

Embodiment 11. The method of any one of embodiments 1-8, wherein the amount of the mTOR inhibitor in the mTOR inhibitor nanoparticle composition is from about 45 mg/m² to about 75 mg/m².

Embodiment 12. The method of any one of embodiments 1-8, wherein the amount of the mTOR inhibitor in the mTOR inhibitor nanoparticle composition is from about 75 mg/m² to about 100 mg/m².

Embodiment 13. The method of any one of embodiments 1-12, wherein the mTOR inhibitor nanoparticle composition is administered weekly, once every 2 weeks, or once every 3 weeks.

Embodiment 14. The method of any one of embodiments 1-12, wherein the mTOR inhibitor nanoparticle composition is administered 2 out of every 3 weeks.

Embodiment 15. The method of any one of embodiments 1-12, wherein the mTOR inhibitor nanoparticle composition is administered 3 out of every 4 weeks.

Embodiment 16. The method of any one of embodiments 1-15, wherein the average diameter of the nanoparticles in the composition is no greater than about 200 nm.

Embodiment 17. The method of any one of embodiments 1-16, wherein the weight ratio of the albumin to the mTOR inhibitor in the nanoparticle composition is no greater than about 9:1.

Embodiment 18. The method of any one of embodiments 1-17, wherein the nanoparticles comprise the mTOR inhibitor associated with the albumin.

Embodiment 19. The method of embodiment 18, wherein the nanoparticles comprise the mTOR inhibitor coated with the albumin.

Embodiment 20. The method of any one of embodiments 1-19, wherein the mTOR inhibitor nanoparticle composition is administered intravenously, intraarterially, intraperitoneally, intravesicularly, subcutaneously, intrathecally, intrapulmonarily, intramuscularly, intratracheally, intraocularly, transdermally, orally, or by inhalation.

Embodiment 21. The method of embodiment 20, wherein the mTOR inhibitor nanoparticle composition is administered intravenously.

Embodiment 22. The method of any one of embodiments 1-21, wherein the amount of the anti-VEGF antibody is from about 1 mg/kg to about 5 mg/kg.

Embodiment 23. The method of any one of embodiments 1-21, wherein the amount of the anti-VEGF antibody is from about 5 mg/kg to about 10 mg/kg.

Embodiment 24. The method of any one of embodiments 1-21, wherein the amount of the anti-VEGF antibody is from about 10 mg/kg to about 15 mg/kg.

Embodiment 25. The method of any one of embodiments 1-21, wherein the amount of the anti-VEGF antibody is from about 15 mg/kg to about 20 mg/kg.

Embodiment 26. The method of any one of embodiments 1-25, wherein the anti-VEGF antibody is administered intravenously, intraarterially, intraperitoneally, intravesicularly, subcutaneously, intrathecally, intrapulmonarily, intramuscularly, intratracheally, intraocularly, transdermally, orally, or by inhalation.

Embodiment 27. The method of embodiment 26, wherein the anti-VEGF antibody is administered intravenously.

Embodiment 28. The method of embodiment 27, wherein the amount of the anti-VEGF antibody is about 10 mg/kg, and wherein the anti-VEGF antibody is administered once every two weeks.

Embodiment 29. The method of any one of embodiments 1-27, wherein the anti-VEGF antibody is administered weekly.

Embodiment 30. The method of any one of embodiments 1-27, wherein the anti-VEGF antibody is administered once every two weeks.

Embodiment 31. The method of any one of embodiments 1-27, wherein the anti-VEGF antibody is administered once every three weeks.

Embodiment 32. The method of any one of embodiments 1-31, wherein the FOLFOX regimen is FOLFOX4 or FOLFOX6.

Embodiment 33. The method of any one of embodiments 1-31, wherein the FOLFOX regimen is a modified FOLFOX4 or a modified FOLFOX6 regimen.

Embodiment 34. The method of embodiment 32 or 33, wherein the FOLFOX regimen is FOLFOX4, and wherein the anti-VEGF antibody is administered intravenously, once every two weeks with an amount of about 10 mg/kg.

Embodiment 35. The method of embodiment 33, wherein the FOLFOX regimen is a modified FOLFOX6, and wherein the anti-VEGF antibody is administered intravenously, once every two weeks with an amount of about 10 mg/kg.

Embodiment 36. The method of any one of embodiments 1-35, wherein the mTOR inhibitor and the anti-VEGF antibody and/or at least a portion of the FOLFOX regimen are administered sequentially to the individual.

Embodiment 37. The method of any one of embodiments 1-35, wherein the anti-VEGF antibody and at least a portion of the FOLFOX regimen are administered sequentially to the individual.

Embodiment 38. The method of any one of embodiments 1-35, wherein the mTOR inhibitor and the anti-VEGF antibody and/or at least a portion of the FOLFOX regimen are administered simultaneously to the individual.

Embodiment 39. The method of any one of embodiments 1-35, wherein the anti-VEGF antibody and at least a portion of the FOLFOX regimen are administered simultaneously to the individual.

Embodiment 40. The method of any one of embodiments 1-35, wherein the mTOR inhibitor and the anti-VEGF antibody and/or at least a portion of the FOLFOX regimen are administered concurrently to the individual.

Embodiment 41. The method of any one of embodiments 1-35, wherein the anti-VEGF antibody and at least a portion of the FOLFOX regimen are administered concurrently to the individual.

Embodiment 42. The method of any one of embodiments 1-41, wherein the individual is human.

Embodiment 43. The method of any one of embodiments 1-42, further comprising selecting the individual for treatment based on the presence of at least one mTOR-activation aberration or the MSI status.

Embodiment 44. The method of embodiment 43, wherein the mTOR-activating aberration comprises a mutation in an mTOR-associated gene.

Embodiment 45. The method of embodiment 43 or 44, wherein the mTOR-activating aberration is in at least one mTOR-associated gene selected from the group consisting of AKT1, FLT-3, MTOR, PIK3CA, PIK3CG, TSC1, TSC2, RHEB, STK11, NF1, NF2, TP53, FGFR4, BAP1, KRAS, NRAS and PTEN.

Embodiment 46. The method of embodiment 45, wherein the mTOR-activating aberration is in PTEN.

Embodiment 47. The method of any one of embodiments 1-46, further comprising assessing a mTOR-activating aberration in the individual.

Embodiment 48. The method of embodiment 47, wherein the mTOR-activating aberration is assessed by gene sequencing or immunohistochemistry.

Embodiment 49. The method of any one of embodiments 1-48, further comprising selecting the individual for treatment based on at least one biomarker indicative of favorable response to treatment with an anti-VEGF antibody.

Embodiment 50. The method of any one of embodiments 1-49, further comprising selecting the individual for treatment based on at least one biomarker indicative of favorable response to treatment with FOLFOX.

Embodiment 51. The method of any one of embodiments 1-50, wherein the colon cancer is advanced.

Embodiment 52. The method of any one of embodiments 1-51, wherein the colon cancer is malignant.

Embodiment 53. The method of any one of embodiments 1-52, wherein the colon cancer is metastatic.

Embodiment 54. The method of any one of embodiments 1-53, wherein the colon cancer is stage I, II, III, or IV cancer.

Embodiment 55. The method of any one of embodiments 1-54, wherein the colon cancer is characterized with a genomic instability.

Embodiment 56. The method of embodiment 55, wherein the genomic instability comprises a microsatellite instability (MSI), a chromosomal instability (CIN) and/or a CpG island methylator phenotype (CIMP).

Embodiment 57. The method of embodiments 1-56, wherein the colon cancer is characterized with an alteration of a pathway, wherein the alteration of a pathway comprises PTEN, TP53, BRAF, PI3CA or APC gene inactivation, KRAS, TGF-□, CTNNB, Epithelial-to-mesenchymal transition (EMT) genes or WNT-signaling activation, and/or MYC amplification.

Embodiment 58. The method of any one of embodiments 1-57, wherein the colon cancer is classified under the colon cancer subtype (CCS) system as CCS1, CCS2, or CCS3.

Embodiment 59. The method of any one of embodiments 1-58, wherein the colon cancer is classified under colorectal cancer assigner (CRCA system) as stem-like, goblet-like, inflammatory, transit-amplifying, or enterocyte subtype.

Embodiment 60. The method of any one of embodiments 1-59, wherein the individual has been previously treated with chemotherapy, radiation or surgery.

Embodiment 61. The method of any one of embodiments 1-59, wherein the individual has not been previously treated.

Embodiment 62. The method of any one of embodiments 1-60, wherein the method is used as an adjuvant treatment.

### EXAMPLES

### Example 1. Use of Nab-Rapamycin in Combination with FOLFOX and Bevacizumab as First-Line Therapy in Patients with Advanced or Metastatic Colorectal Cancer

ABI-009 (“nab-rapamycin”) is rapamycin protein-bound nanoparticles for injectable suspension (albumin bound). Upon the combination with FOLFOX and bevacizumab, it enhances therapeutic efficacy and/or reduces normal tissue toxicity in advanced or metastatic colorectal cancer. This study is a prospective phase I/II, single arm, open-label, multi-institutional study to identify the recommended phase II dose (RP2D) and determine the efficacy and safety profile of ABI-009 administered as a first-line therapy in combination with FOLFOX and bevacizumab in patients with advanced or metastatic colorectal cancer.

### Combination Therapy Administration

Patients receive ABI-009 at different dosages as described in Table 3 by IV infusion over 30 minutes weekly for 3 weeks followed by a week of rest (qw3/4, 28-day cycle). Bevacizumab with a dose of 10 mg/kg and mFOLFOX6 are administered every 2 weeks, starting Cycle 1, Day 1.

Modified FOLFOX6 regimen is as following: oxaliplatin 85 mg/m² IV with leucovorin (LV) 400 mg/m² IV over 2 hours plus 5-FU 400 mg/m² IV bolus and 2,400 mg/m² continuous infusion over 46 hours every 2 weeks. Dose modifications of each agent in FOLFOX may be made independently based on the specific types of toxicities observed. Bevacizumab may be skipped or discontinued for bevacizumab-related toxicities, but the dose is not reduced.

Patients continue with the combination therapy 1) until disease progression, 2) until unacceptable toxicity, 3) until the time when the investigator believes the patient is no longer benefiting from therapy, or 4) at the patient's discretion. Patients who remain on treatment for more than 6 months may be switched to mFOLFOX and bevacizumab every 3 weeks and ABI-009 given weekly for 2 weeks followed by a week of rest (qw2/3, 21-day cycle) at the discretion of the investigator.

### Objectives and Endpoints

The phase I study is performed to determine the RP2D of ABI-009 in combination with FOLFOX and bevacizumab and to evaluate the preliminary efficacy and the safety of ABI-009 in combination with FOLFOX and bevacizumab at the RP2D. The phase II study is performed to further evaluate the efficacy and safety of ABI-009 in combination with FOLFOX and bevacizumab at the RP2D, as well as the toxicity profile of ABI-009 with the combination therapy at the RP2D. The serum proteomic profiles of patients treated with the combination therapy is also determined.

The primary endpoints used in phase I are dose-limiting-toxicities (DLTs) and maximum-tolerated dose (MTD) of ABI-009 in combination with FOLFOX and bevacizumab. The secondary endpoints used in phase I are a) safety profile of dose cohorts analyzed separately and together; and b) disease control rate (DCR) of dose cohorts analyzed separately and together.

In phase II, the progression-free survival (PFS) at 6 months of ABI-009 (in combination with FOLFOX and bevacizumab) at the RP2D and all dose cohorts are assessed as the primary endpoints. Overall response rate (ORR), duration of response (DOR), median PFS, and disease control rate (DCR) at the RP2D and all dose cohorts and safety at RP2D, including patients from phase I are used as secondary endpoints.

Furthermore, pre-treatment tumor biopsy (e.g., archived samples or fresh tissues within 3 months prior to the treatment) are performed on all patients from phase I and II to assess baseline biomarker and mutational analysis, including but not limited to PTEN loss evaluation, Ras mutational status, mTOR pathway markers (including, but not limited to S6K, 4EBP1). Blood samples at different time points (e.g., pretreatment, post-treatment (such as at Day 1 of the third cycle, i.e., C3-D1), and upon disease recurrence) are collected from all patients from phase I and II. Molecular analysis of circulating DNA assay using next generation sequencing are performed to assess changes over time as response to the combination therapy with regard to the prevalence of mutations identified in the baseline tumor samples. For example, nucleic acids extracted from blood are used to investigate whether circulating tumor nucleic acids are associated with disease recurrence. Pharmacokinetic and/or pharmacodynamic information of ABI-009 of all patients from phase I and II are studied to assess the relationships with the safety and/or efficacy endpoints.

### Study Design and Dose-Finding Rules

The study is conducted in compliance with International Conference on Harmonisation (ICH) Good Clinical Practices (GCPs).

In the dose-finding portion of the study (phase I), dose levels of ABI-009 is tested in cohorts of 3 patients each using the 3+3 dose-finding design as shown in Table 3.

TABLE 3

| Dose-levels | ABI-009 in mg/m² |
|---|---|
| −2 | 10 |
| −1 | 20 |
| 1 | 30 |
| 2 | 45 |
| 3 | 60 |

Escalation to the next dose level with a new cohort of 3 patients occurs after no DLT is observed in the 1st treatment cycle of 4 weeks. No intra-patient dose escalation is allowed. If a DLT occurs in a cohort, additional 3 patients will be recruited to the cohort. If no further DLTs occur, then a new cohort of 3 patients at the next higher dose level can be enrolled. If two or more out of six patients at a specific dose level experience a DLT, then that cohort will be closed to further enrollment and 3 patients will be enrolled at the next lower dose level, and so on.

The MTD is the highest dose level in which less than one patient has a DLT. The RP2D is identified based of the totality of safety and efficacy data.

### Patients

Up to 42 evaluable patients are enrolled in the study, with up to 18 in the dose-finding phase I portion and 24 additional patients in phase II (total N=30 in phase II, including patients from phase I at the RP2D).

In phase I, it is estimated that a maximum of up to 18 patients are required to achieve the MTD; however, MTD could be reached with as few as 9 patients.

In phase II, 24 additional patients are enrolled at the RP2D, for a total of 30 patients (including 6 patients from phase I at the RP2D).

A patient is eligible for inclusion in this study only if all of the following criteria are met at screening. 1. The patient with histologically confirmed advanced or metastatic colorectal cancers for whom chemotherapy is indicated. 2. The patient must not have had prior chemotherapy for advanced or metastatic disease, although patients could have received adjuvant chemotherapy or adjuvant chemo-radiotherapy. 3. The patient must have at least 1 measurable site of disease according to RECIST v1.1 that has not been previously irradiated. However, if the patient has had previous radiation to the marker lesion(s), there must be evidence of progression since the radiation. 4. The patient must be 18 years or older, with Eastern Cooperative Oncology Group (ECOG) performance status 0, 1, or 2.5. The patient must not have been previously treated with an mTOR inhibitor. 6. The patient must have adequate liver function, which includes a) total bilirubin is or is less than 1.5× upper limit of normal (ULN) mg/dL; and b) aspartate aminotransferase (AST) and alanine aminotransferase (ALT) is or is less than 2.5×ULN (less than 5×ULN if the patient has liver metastases). 7. The patient must have adequate renal function, which includes that the level of serum creatinine is or is more than 2×ULN or creatinine clearance is more than 50 cc/hr. 8. The patient must have adequate biological parameters, which include: a) absolute neutrophil count (ANC) is or is more than 1.5×10⁹/L; b) platelet count is or is more than 100,000/mm³ (100×10⁹/L); and c) the level of hemoglobin is or is more than 9 g/dL. 9. The level of fasting serum triglyceride is or is less than 300 mg/dL; the level of fasting serum cholesterol is or is less than 350 mg/dL. 10. The internationalized normalized ratio (INR) and the partial thromboplastin time (PTT) is less than 1.5×ULN (anticoagulation is allowed if target INR is less than 1.5 on a stable dose of warfarin or on a stable dose of LMW heparin for more than 2 weeks at time of enrollment). 11. At least four weeks have passed since any major surgery, completion of radiation, or completion of all prior systemic anticancer therapy (adequately recovered from the acute toxicities of any prior therapy) when the treatment is initiated.

### Duration of Treatment and Study Participation

This study takes approximately 36 months from first patient enrolled to last patient follow-up, including approximately 24 months of enrollment period, an about 6 months of treatment (or until treatment is no longer tolerated).

End of Treatment (EOT) for a patient is defined as the date of the last dose of ABI-009. End of Treatment Visit for a patient is when safety assessments and procedures are performed after the last treatment, which must occur within 1 week (±3 days) after the last dose of ABI-009.

The End of Study (EOS) is defined as either the date of the last visit of the last patient to complete the study, or the date of receipt of the last data point from the last patient that is required for analysis, such as these described herein.

Follow-up period is the on-study time period after the EOT Visit. All patients that discontinue the combination therapy and have not withdrawn full consent to participate in the study continue in the follow-up phase for survival and the initiation of another anticancer therapy. Follow up continues approximately every 12 weeks (±3 weeks), until death, withdrawal of consent, or the study closes, whichever is the earliest. This evaluation may be made by record review and/or telephone contact.

### Key Efficacy Assessments

Efficacy is assessed by using CT scans and RECIST (version 1.1) criteria. Standard RECIST (version 1.1) definitions of Stable, Progressive disease and Responses are used. Only RECIST (version 1.1) criteria is used to assess response. PET is used for qualitative purposes only.

For the phase II portion of the study the primary endpoint is progression-free survival (PFS) at 6 months after treatment. Progression-free survival is defined as the time from the first day of combination therapy administration to disease progression or death due to any cause. In addition to an exact binomial test for 6-month, PFS is analyzed using Kaplan-Meier methods and summarized by presenting the 25th, 50th, and 75th percentiles of PFS, and associated 2-sided 95% confidence intervals.

The ORR and DCR are reported along with a 95% confidence interval computed by the Clopper-Pearson method.

### Key Safety Assessments

Safety assessments consist of monitoring and recording all adverse events and serious adverse events, the regular monitoring of hematology, blood chemistry and urine values, regular measurement of vital signs and the performance of physical examinations.

Safety and tolerability are assessed according to the NCI CTCAE, version 4.0.

For the phase I portion of the study the primary endpoint is safety as summarized descriptive statistics.

### Example 2: Patient with Stage IVB Metastatic Colorectal Cancer Treated with ABI-009

A patient who is a 61-year old male and was diagnosed with stage IVB metastatic colorectal cancer in May of 2018, with pancreatic, lung and liver metastases and ongoing weight loss since February of 2018. The patient also had a long history of smoking (>40 years). The patient received the experimental therapeutic ABI-009 at 30 mg/m² intravenously, along with a standard of care of modified FOLFOX6 plus bevacizumab (doses: bolus 5FU at 400 mg/m², 5FU continuous 2400 mg/m², oxaliplatin at 85 mg/m², bevacizumab at 5 mg/kg). This patient received 3 full doses of each therapeutic every other week, within 5 weeks in July and August of 2018. The patient presented for a subsequent treatment visit and reported anorexia and ongoing weight loss (140 lb at the start of the treatment and 123 lb at the time of visit; 17 lb [12%] loss of body weight). The patient was hospitalized for failure to thrive in September of 2018, then was released for palliative care at home with tube feedings.

In October of 2018, the patient was reported to have recovered from the episode and was eating better and started to gain weight. The patient has received no additional anti-cancer therapy since the last study dose on in August of 2018. The patient had a CT scan and a physical evaluation in November of 2018, 2.5 months after the last dose of therapy. This evaluation revealed that compared to baseline CT scans conducted in July prior to the treatment, there has been interval decrease in size of hepatic and pancreatic metastatic lesions. In addition, there has been a decrease in size of the left common iliac chain lymph node, as well as a few pulmonary nodules. Surprisingly, the dominant nodule in the right perihilar region of the lung (8.7×6.0 cm) appeared as a cavitary lesion, with significant necrosis despite no therapy for this patient's disease from time of the last dose in August of 2018, 2.5 months earlier.

The patient reported feeling well with body weight gained 15.2 lb as measured from the start of hospitalization in September and tumor biomarker carcinoembryonic antigen (CEA) dropped nearly 3-fold (from 14.4 to 5.1 ng/mL) below the levels of baseline screening, when the patient presented for therapy. It is important to note that the normal level of CEA is <5 ng/mL for smokers. The treating physician reported that he has not seen this kind of response in patients receiving just the combination of FOLFOX and bevacizumab.

What is claimed is:

1. A method of treating a colon cancer in an individual, comprising administering to the individual: a) an effective amount of a composition comprising nanoparticles comprising an mTOR inhibitor and an albumin, b) an effective amount of anti-VEGF antibody, c) a therapeutically effective FOLFOX regimen.

2. The method of claim 1, wherein the colon cancer comprises an mTOR-activation aberration.

3. The method of claim 2, wherein the mTOR-activation aberration comprises a PTEN aberration.

4. The method of claim 1, wherein the mTOR inhibitor is a limus drug.

5. The method of claim 4, wherein the limus drug is rapamycin.

6. The method of claim 1, wherein the anti-VEGF antibody is bevacizumab.

7. The method of claim 1, wherein the amount of the mTOR inhibitor in the mTOR inhibitor nanoparticle composition is from about 10 mg/m² to about 30 mg/m².

8. The method of claim 1, wherein the mTOR inhibitor nanoparticle composition is administered weekly, once every 2 weeks, or once every 3 weeks.

9. The method of claim 1, wherein the average diameter of the nanoparticles in the composition is no greater than about 200 nm.

10. The method of claim 1, wherein the weight ratio of the albumin to the mTOR inhibitor in the nanoparticle composition is no greater than about 9:1.

11. The method of claim 1, wherein the nanoparticles comprise the mTOR inhibitor coated with the albumin.

12. The method of claim 1, wherein the mTOR inhibitor nanoparticle composition is administered intravenously.

13. The method of claim 1, wherein the amount of the anti-VEGF antibody is from about 1 mg/kg to about 5 mg/kg.

14. The method of claim 1, wherein the anti-VEGF antibody is administered intravenously.

15. The method of claim 14, wherein the amount of the anti-VEGF antibody is about 5 mg/kg to about 10 mg/kg, and wherein the anti-VEGF antibody is administered once every two weeks.

16. The method of claim 1, wherein the FOLFOX regimen is FOLFOX4 or FOLFOX6.

17. The method of claim 1, wherein the FOLFOX regimen is a modified FOLFOX4 or a modified FOLFOX6 regimen.

18. The method of claim 1, wherein the individual is human.
